(19) United States
(12) Patent Application Publication
(10) Pub. No.: US 2022/0211796 A1
(43) Pub. Date: Jul. 7, 2022

(54) PEPTIDE ANTIBIOTICS AND METHODS OF USE THEREOF

(71) Applicant: Trustees of Tufts College, Medford, MA (US)

(72) Inventors: Krishna KUMAR, Medford, MA (US); Emel ADALIGIL, Medford, MA (US)

(21) Appl. No.: 17/611,720

(22) PCT Filed: May 15, 2020
(86) PCT No.: PCT/US2020/033275

§ 371 Date: Nov. 16, 2021

Related U.S. Application Data
(60) Provisional application No. 62/848,957, filed on May 16, 2019.

(51) Int. Cl. A61K 38/00; A61P 31/04; C07K 7/06; C07K 7/08

(52) U.S. Cl. CPC A61K 38/00; C07K 7/08; C07K 7/06; A61P 31/04

(57) ABSTRACT

The invention features peptide antibiotic compositions and methods of using such compositions for the treatment of bacterial infections (e.g., vancomycin resistant infections).

### STATEMENT OF RIGHTS TO INVENTIONS MADE UNDER FEDERALLY SPONSORED RESEARCH

This invention was made with government support under grant GM065500 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Nov. 15, 2021, is named 51415-003002_Sequence_Listing_11_15_21_ST25 and is 102,989 bytes in size.

### BACKGROUND OF THE INVENTION

Bacterial resistance to antibiotics in clinical use is a clear and present danger to public health. Because development of resistance is inevitable, new strategies for discovery of compounds with antimicrobial activity are urgently needed. Staphylococcus aureus is a leading cause of community-associated and nosocomial infections, and its ability to become resistant to existing therapeutics poses a significant concern. Although vancomycin, often referred to as ‘the antibiotic of last resort’, has been effective in the treatment of infections caused by S. aureus and methicillin-resistant S. aureus (MRSA) for more than three decades, emergence of vancomycin-resistant strains underscore the critical need for expansion of the pipeline of lead compounds. Several approaches are currently being pursued in an attempt to develop compounds that are active against resistant bacteria, such as modification of existing antibiotics, finding new leads from nature, and expansion of new synthetic classes through rational design. The mode of action of antibiotics have typically consisted of the selective inhibition of bacterial enzymes, exploiting differential structures of the bacterial versus the human ribosomes, selective membrane disruption, and the binding of DNA or RNA sequences that are unique to bacteria. Among these approaches, targeting bacterial cell wall biosynthesis has been a fruitful line of investigation for the discovery of several classes of antibiotics.

Peptide based therapeutics afford a high level of specificity. However, the use of these compounds is limited because of hydrolytic cleavage by proteases, possible induction of a vigorous humoral immune response and administration mostly as injectables.

### SUMMARY OF THE INVENTION

As described below, the present invention features peptide antibiotic compositions and methods of using such compositions for the treatment of bacterial infections (e.g., vancomycin resistant infections). In particular embodiments, the invention features peptides composed of D-amino acids, which are metabolically stable, are either less or non-immunogenic, and short sequences (<12 residues) have been known to be absorbed through the gut making them viable oral drugs. These favorable features make them a particularly attractive class of new therapeutics.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a peptide analog retains the biological activity of a corresponding naturally-occurring peptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include bacterial infections (e.g., antibiotic resistant infections).

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a peptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide or fragment thereof (e.g., peptide) of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1. a Schematic representation of the mirror image phage display concept: the phage display peptide library is screened for identification of L-peptides that bind the antipode of the target molecule. Due to symmetry relations, L-peptides identified through phage display can be synthesized in the D-form that would now bind the original L-target. b Chemical structures of cephalosporin and N-acyl-D-Ala-D-Ala terminus of cell wall peptidoglycan precursor showing structural similarity as well as N-acyl-D-Ala-D-Lac terminus of cell wall peptidoglycan precursor of vancomycin-resistant bacteria. c Structures of target molecules used in phage display screenings, Cep-1, Ala-2, and Lac-4.

FIG. 2. Peptides identified from phage display screening against Cep-1 and Ala-2 were identical. a Sequences of peptides are shown with phage display libraries isolated from libraries depicted in the table. b Phage-ELISA showing the binding differences of identical peptide sequences from each target molecule. Data are shown as mean±standard deviation of three independent experiments.

FIG. 3. Phage display of bicyclic peptides. a Structure and diversity of two bicyclic peptide libraries used in the phage display screening. b Schematic representation of bicyclization reaction on phage protein pIII with 1,3,5-tris(bromomethyl)benzene (TBMB) through three cysteine residues in peptides expressed on the surface.

FIG. 4. Properties of antibiotic D-peptides. a The binding capabilities of potent peptides (MIC value of <256 μg/ml) from phage display screening with linear 12-mer, linear 7-mer and cyclic 7-mer libraries against target molecules, Cep-1 and Ala-2, by phage-ELISA. A control peptide containing “HPQ” consensus sequence that specifically binds to streptavidin was also included. The experiments were performed in triplicate and data are presented as mean±standard deviation. b Time-dependent killing kinetics of S. aureus in presence of P14 and P15 compared to vancomycin at 10×MIC; both peptides show bactericidal activity similar to vancomycin. Data are shown as mean±SD (n=3). c Accumulation of the soluble cell wall precursor UDP-MurNAc-pentapeptide of S. aureus after incubation with P14 (red), P15 (green) and vancomycin (blue). d MALDI-TOF mass spectrum confirms accumulation of UDP-MurNAc-pentapeptide of S. aureus (MW=1149.52).

FIG. 5. Peptides identified from phage display screening against target molecules Lac-4 and Ala-2 with identical sequences. a Peptides with their sequences and which commercially available phage display libraries they were identified. b Peptides showed different binding affinities for target molecules, Lac-4 and Ala-2, in a phage-ELISA assay. Data are shown as mean±standard deviation (n=3).

FIG. 6. Toxicity of D-peptides. a Percentage hemolysis of hRBCs by P14 and P15 with melittin (100%) and vancomycin (0%) as controls. b Percentage hemolysis of hRBCs by P18. c Cytotoxicity of potent D-peptides, P14, P15 and P18, on HeLa cells compared to non-toxic vancomycin and toxic melittin. Data are depicted as mean±S.D. (n=3).

FIG. 7. a Drug-like properties of potent D-peptide antibiotics. P14, P15 and P18, identified from phage display screening against vancomycin-sensitive, P14 and P15, and vancomycin-resistance bacteria target molecules, P18. b Chemical structures of bicyclic D-peptides, P14, P15 and P18.

FIG. 8 provides a schematic for the experimental scheme.

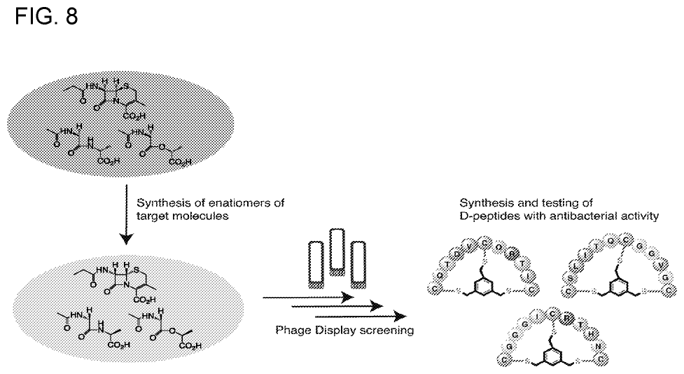

FIG. 9 shows the synthesis of construct Cep-1 that contains the enantiomeric cephalosporin scaffold. Reagents and conditions. a PhtCO₂Et, Na₂CO₃, H₂O, rt, 2 h, 45%; b BnBr, Et₃N, DMF, rt, 6 h, 60%; c DBU (cat.) in CH₂Cl₂, rt, 90 min, 64%; d SO₂Cl₂ (1 equiv.), CCl₄, 0° C., 30 min; e SnCl₂, THF, rt, 2 h reflux, 30% over 2 steps; f 03, Me₂CO, −78° C., quantitative yield; g pTSA (cat.), DMF, 100° C., 90 min, 52%; h 1M N₂H₄ in DMF, −78° C. to rt, 30 min, 40%; i NHS-PEG₁₂-Biotin, DIPEA, DMF, 0° C., overnight, 65%; j AlCl₃, PhOMe, CH₂Cl₂, CH₃NO₂; 0° C. to rt, 8 h, 28%

FIG. 10 is a ¹H NMR spectrum of compound 10, Cep-1.

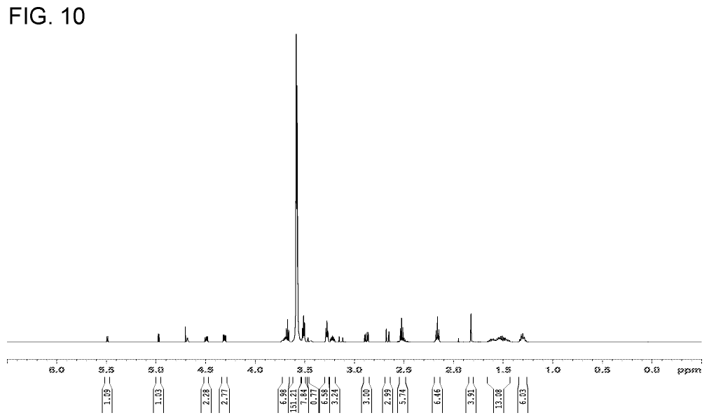

FIG. 11 is a ¹³C NMR spectrum of compound 10, Cep-1.

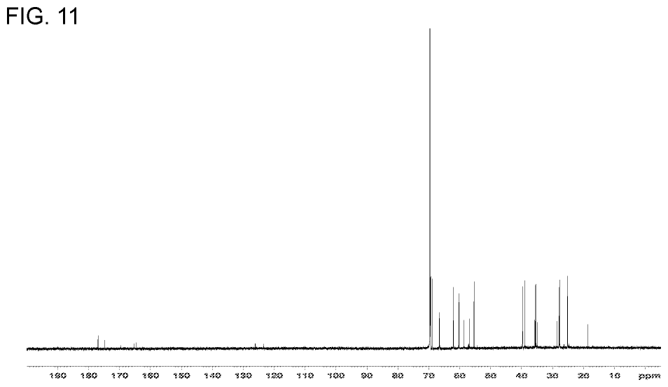

FIG. 12 is a RP-HPLC chromatogram of Cep-1. Linear gradient was applied from 20% to 40% solvent B over 40 min.

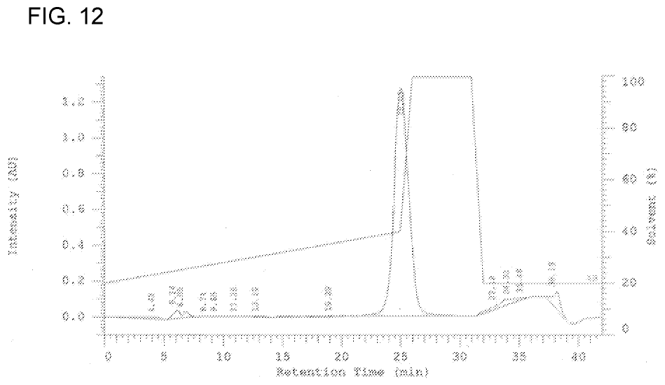

FIG. 13 is a MALDI TOF-MS spectrum of pure Cep-1. Calculated mass [M+Na+H]⁺, 1062.62, observed mass 1062.42.

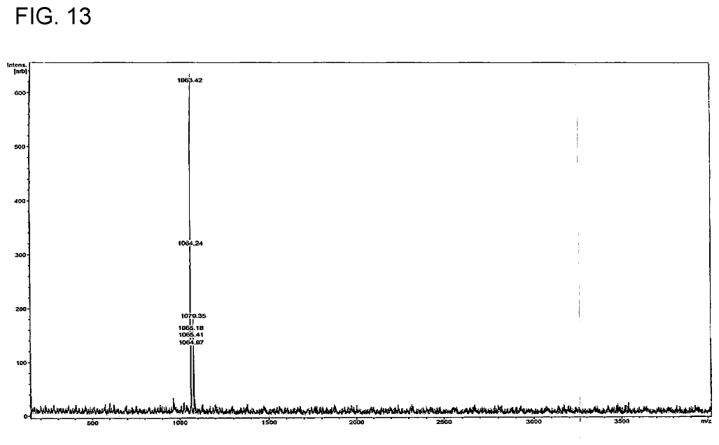

FIG. 14 is a RP-HPLC chromatogram of Ala-2. Linear gradient was applied from 5% to 20% solvent B over 30 min.

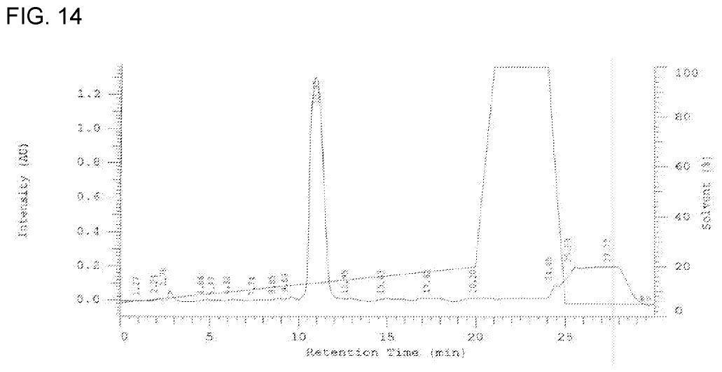

FIG. 15 is a MALDI TOF-MS spectrum of pure Ala-2. Calculated mass [M+H]⁺, 962.46, observed mass 962.121.

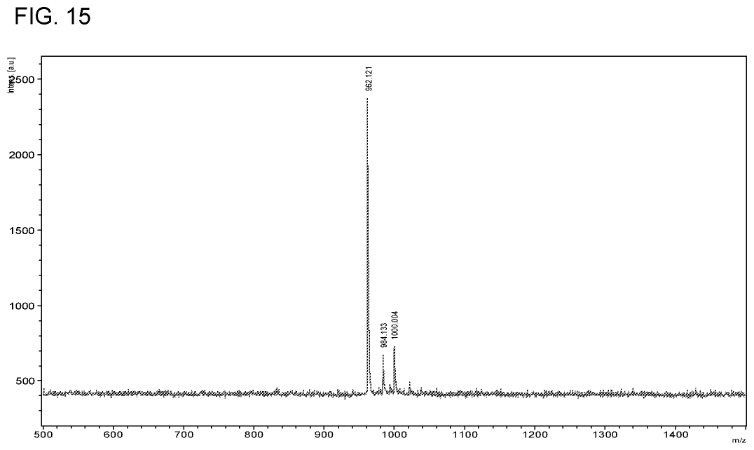

FIG. 16 is a RP-HPLC chromatogram of Ala-3. Linear gradient was applied from 5% to 20% solvent B over 30 min.

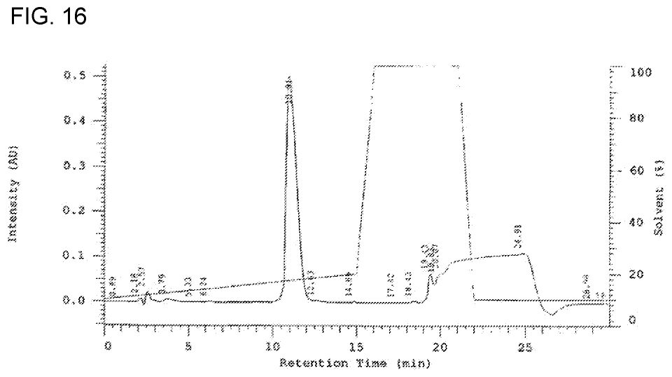

FIG. 17 is a MALDI TOF-MS spectrum of pure Ala-3. Calculated mass [M+H]⁺, 962.46, observed mass 962.121.

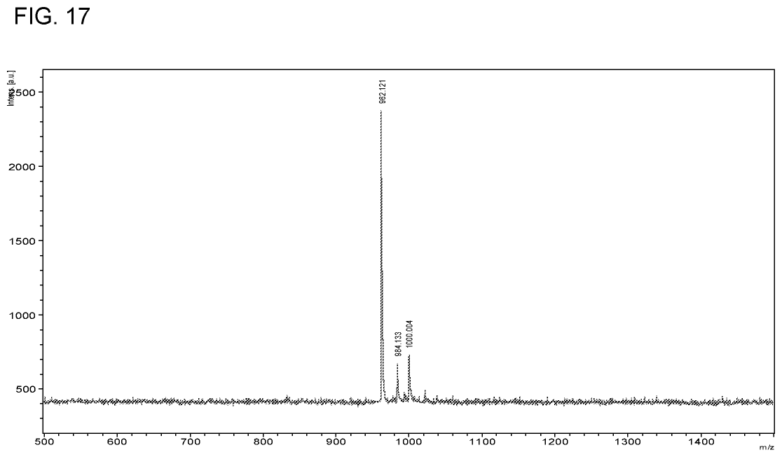

FIG. 18 is a RP-HPLC chromatogram of Lac-4. Linear gradient was applied from 5% to 20% solvent B over 30 min.

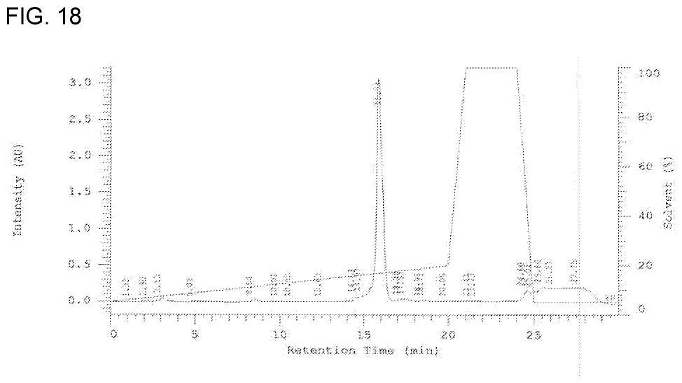

FIG. 19 is a MALDI TOF-MS spectrum of pure Lac-4. Calculated mass [M+H]⁺, 963.26 [M], observed mass 963.115.

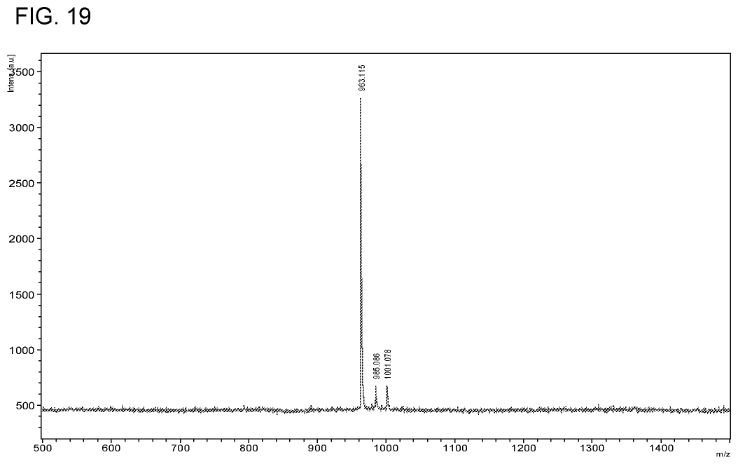

FIG. 20 is a RP-HPLC chromatogram of P14. Linear gradient was applied from 5% to 30% solvent B over 30 min.

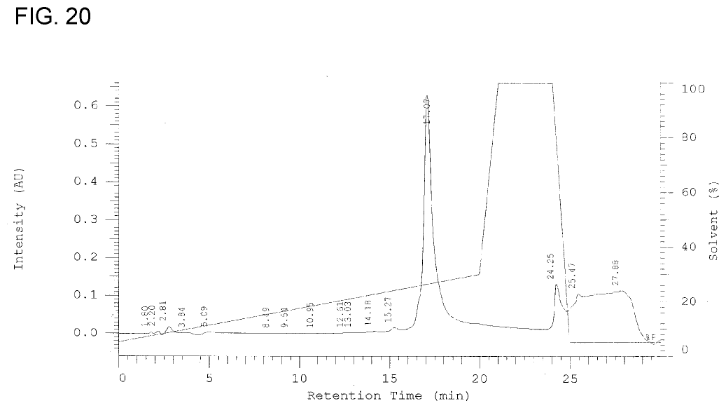

FIG. 21 is a MALDI TOF-MS spectrum of pure P14. Calculated mass [M+H]⁺, 1711.17 [M], observed mass 1711.716.

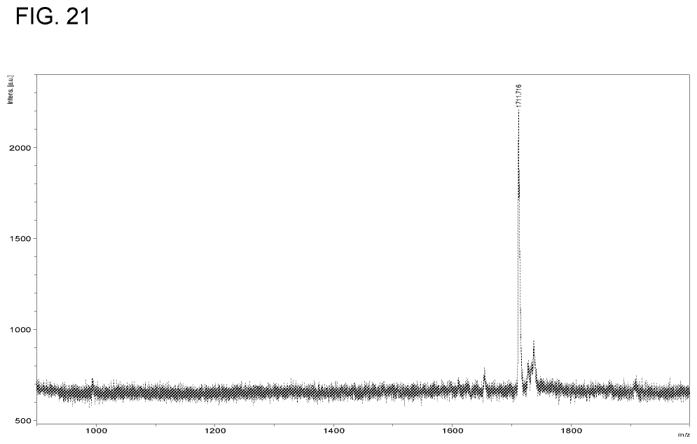

FIG. 22 is a RP-HPLC chromatogram of P15 Linear gradient was applied from 5% to 30% solvent B over 30 min.

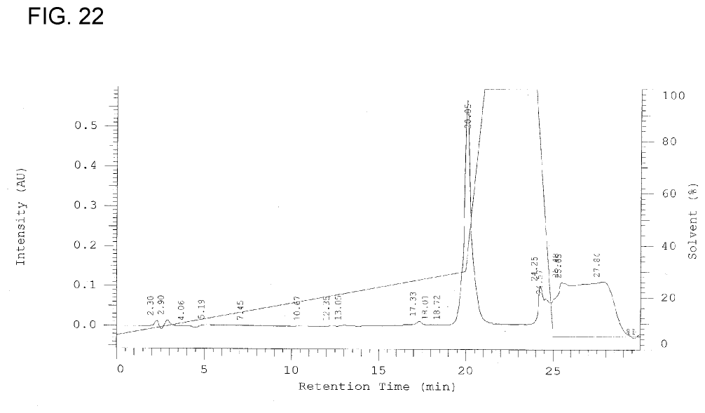

FIG. 23 is a MALDI TOF-MS spectrum of pure P15. Calculated mass [M+H]⁺, 1582.69 [M], observed mass 1582.92.

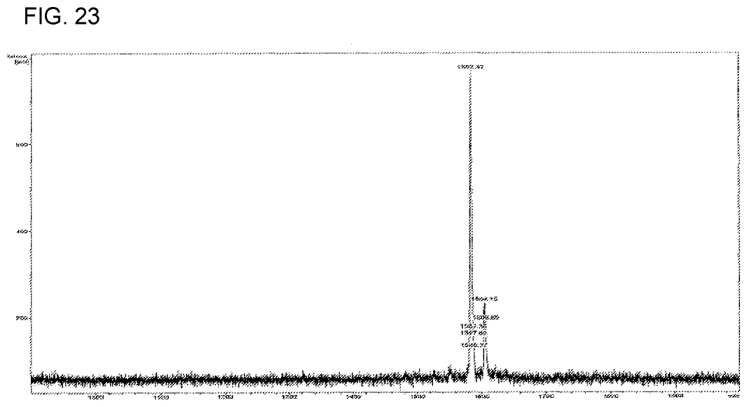

FIG. 24 is a RP-HPLC chromatogram of P18 Linear gradient was applied from 20% to 40% solvent B over 30 min.

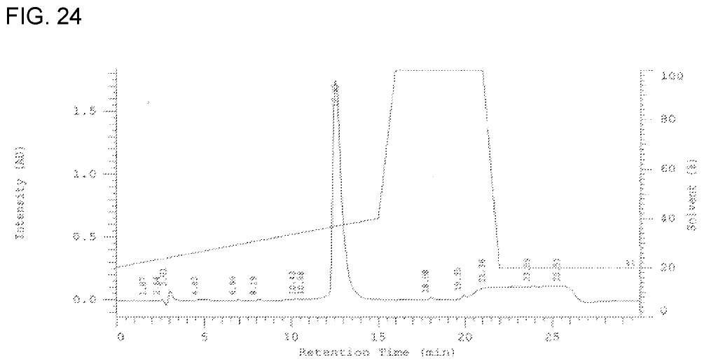

FIG. 25 is a MALDI TOF-MS spectrum of pure P18. Calculated mass [M+H]⁺, 1562.61 [M], observed mass 1562.29.

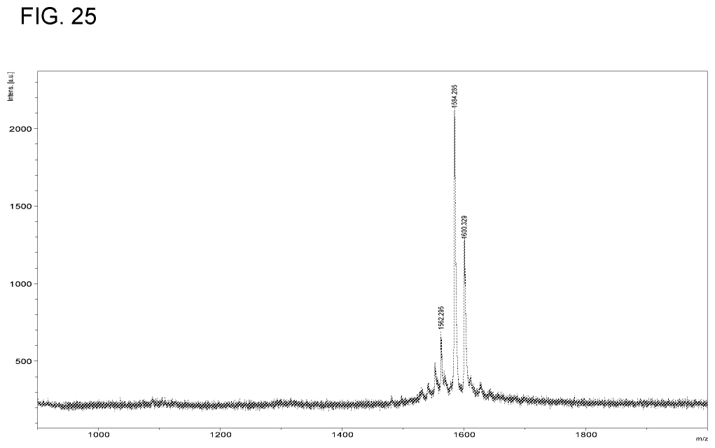

FIG. 26 shows the Structural similarities between peptidoglycan cell wall termini L-Lys-D-Ala-D-Ala and penicillin. Calculated structural overlays of the best conformations of N-Ac-D-Ala-D-Ala and Ampicillin (a β-lactam). Longest RMS distance=1.70 Å; shortest 0.29 Å. (Figure is based on the calculations done by Boyd et al J. Med. Chem. 1975, 18, 408-417).

FIG. 27 shows identical peptides isolated from phage display experiments of target molecules Ala-2 and Ala-3. a Sequences of peptides that were selected as well as phage display libraries are shown in the table. b Binding affinities of each peptide for target molecules Ala-2 and Ala-3 were evaluated by phage-ELISA experiments. Data are shown as mean±standard deviation. (n=3).

FIG. 28 shows Hemolytic activity of potent D-peptides showing minimum inhibitory activity less than 256 μg ml⁻¹ against both vancomycin-sensitive and resistant bacteria compared to vancomycin (blue) and melittin (red). All peptides showed no toxicity on red human blood cells. Data are shown as mean±standard deviation (n=3).

FIG. 29 shows Proteolytic and serum stability of potent D-peptides. RP-HPLC analysis of 1 mg/ml solutions of three potent bicyclic D-peptides, P14, P15 and P18, a After incubation with 50 μg/ml of pancreatin, and digestion of samples analyzed after 0, 15, 30, 60, 120, 180, 240, 360 min and overnight. b After incubation with human serum and digestion of samples analyzed after 0, 5, 30, 60, 120, 240 min and overnight.

FIG. 30 is a table showing phage display peptide libraries used during affinity selections.

### DETAILED DESCRIPTION OF THE INVENTION

The present invention features peptide antibiotic compositions and methods of using such compositions for the treatment of bacterial infections (e.g., vancomycin resistant infections).

A paucity of viable programs and pipelines for the discovery of new antibiotics poses a significant public health threat. The emergence of resistant strains against vancomycin is particularly dangerous in hospital settings. Here we report the design of enantiomeric targets based on bacterial cell wall biosynthesis precursors that allow for selection and identification of short linear, cyclic and bicyclic peptides that are composed of D-amino acids. These compounds are active against S. aureus, Methicillin-resistant S. aureus (MRSA), and vancomycin-resistant Enterococci (VRE) that possess moderately high antibacterial activity, and furthermore display no toxicity to both human red blood cells (hRBCs) and mammalian cells (HeLa) at these concentrations. This ‘mirror image phage display’ approach yielded templates that can serve as scaffolds for further improvements in activity based structural modifications. This strategy has potential to provide a new class of antimicrobials that are metabolically stable and have the promise for oral delivery. The use of this platform combined with traditional medicinal chemistry approaches could rapidly yield large numbers of new therapeutic peptides. Peptides of the invention are provided in the following tables.

TABLE1
Peptide sequences identified from screening with
linear 12-mer Phage Display Library, where all
peptides are written from N-terminus to C-terminus
and have a free N-terminus and an amidated C-terminus.
Linear 12-mer Peptide
Sequences

| | | | |
|---|---|---|---|
| ADWYHWRSHSSS | GLHTSATNLYLH | LETRLGTSPGHT | SPDVTRWPYWVI |
| AKDTHTGRMTNW | GLPHAREDYLDL | LPSDYRSRWADP | SSSVTPVSALHG |
| ANVKAFFSTTQV | GLVNLMRPWHLL | MDLGYVEGSARV | SVLSYSVAYSDS |
| APFPSVALKVPL | GMLQHALVPKVW | NGYHPPGFLAPE | SWFSDWDLELHA |
| ASPVNYRDMFSR | GPFVNLRSHFES | NHLSTPVWSITG | SWHHGDGPIWYG |
| ATNAKRTPNTRI | GQSEHHMRVASF | QCFNSVCLHTNP | TGVYWTQLNADS |
| ATWARVDAIARA | GSAPLLTVDTSK | QDIQTTPPKFSV | TQAIDDIIIGRI |
| CLNDSLYCQGMP | GTGLVTLPRLTV | QFDYMRPANDTH | TTVDYFRKVWVV |
| CPLFDSVRCTSK | GTTWVATAGKLI | QGRIDLYGFLSH | TYTANDLHLADL |
| DLMPHKRMIELV | GVGLTTNVTRAG | QITSSHVWDMGH | VEAKCCFSMHKT |
| DRWVARDPASIF | HHHFVTHPAWVL | QLATLHKLSGPT | VFARGQYDAPHP |
| EFHPMPDGFRSR | HKHWSTPEFLSS | QSHYDSHLAMLV | VGSNLRLLHQWK |
| EGNWFLSFHAST | HKTDLWMTNTIK | QVDNTSSIERLR | VHWDFRQWWQPS |
| FAPALSNPPLRD | HRDPHSALTRSW | RKVKRRPRVSNL | VPKSVDNTFYTP |
| FGRMAWTPMAPM | HTSSLWHLFRST | SAGNSANWLLHW | VSHRSTANFIGS |
| FHFPLGMHSRDE | HVVTSSKTAGPA | SFPHFTLRAYAS | VSNFTKPHKPKA |
| FIPFDPMSMRWE | IGRTVPHQDFAR | SGVYKVAYDWQH | WHPRYVVSPLQY |
| FYLPPNASYMSR | IPLGRDGGSYQR | SINGLLSNSHGS | YAQVYSNHGSRI |
| GDSGLVESHRNV | ITGLGSGSSTST | SLDGAGAALRTS | YIGEMDTLPIST |
| GDVSDVWTAANN | KHFPLGMEYLVT | SLTVPYLTSISD | YPDKITWQAPWL |
| GGGLGSLHETSM | KPGDTAMHYFPP | SNSIDKVNRPIN | YSLRSDFLPFAT |
| GGRRCRIKNCYA | | | |

TABLE2
Peptide sequences identified from screening with linear 7-mer
Phage Display Library, where all peptides are written from
N-terminus to C-terminus and have free a N-terminus and an
amidated C-terminus.
Linear 7-mer Peptide
Sequences

| | | | | |
|---|---|---|---|---|
| ADRFQAL | GKDYMGY | LPGSEQR | SADYSAR | VDSRYHP |
| AGKPFHF | GMWHLPQ | LPKMYSQ | SEHNGTQ | VHPLKLI |
| AHGRSRG | GNVGSVR | LPNSAYV | SEVYPQK | VLTRCCG |
| AHTDWFN | GQSEKHL | LPTGHFL | SFFEQVH | VPIYHLT |
| AIDFARN | GSFWHHN | LPVRLDW | SFRIGPA | VPVWALT |
| ALQPQKH | GSPDSEF | LTLGLPY | SFSQNLH | VSGFRID |
| ALSYSRG | GVHREQI | LVMHSEN | SFVSMPE | VSQRTEP |
| APKPIKL | GVMNHTF | LVPSDKL | SHENFTS | VSRANEG |
| APTPGNV | HGGVRLY | MAPTHSI | SHGTWTP | VSRDTPQ |
| AQYVAVG | HIARLSY | MIRGTTV | SLIAHYQ | VTNTPWP |
| ASLSKYS | HLFTTGV | MMVLRNQ | SNMSHAT | VTSPYAF |
| ATYGNLW | HLKHSLL | MPDMTRQ | SPWQYTN | VYPGPSY |
| AVRGYEW | HLNQQNH | NDLMNRA | SQNFVRE | WDPRVNV |
| AYDDWFW | HMGKLNR | NDRLHTR | SSDVPYL | WGRISHV |
| DISRMAT | HVMTKAL | NIGQDMH | SSLRIPV | WPTHYLV |
| DSVETKP | HVRHYSD | NIVSRES | SSNQFHQ | WQEHRDQ |
| DTALHSL | HYIDFRW | NLRLPYI | STKTLPA | WQWPARV |
| DVMMPRH | IPFSFTG | NSIYQAW | STVKYID | WSLSELH |
| ETALIAA | IRIAEPM | NSYDVQA | SWTALGP | WSWGEQK |
| FPAWFSA | ISTPYIG | NTAVPLG | TDEIKLL | YGGAALQ |
| FPITYDF | KCCYTLP | NTVANNY | TGFLVNV | YMLDSTM |
| FSTTHPD | KFYAHLD | PRLPRTR | TMQNIPN | YNISVNK |
| FSYSFQH | KLSMQHR | QLAVAPS | TQTVLGD | YPFFSSM |
| FVRIHDV | KPPPTLD | QLKWYHA | TSQYLMI | YPWFIRA |
| GASESYL | KTALALE | QQTNWSL | TTLLTVS | YPWWNTL |
| GFGYNVQ | KVKKRPD | RHDIRKT | TTQVLEA | YQWELYS |
| GGGHLSR | LAQSSIQ | RPTAHMA | TVNFKLY | YSEPAVT |
| GHRVRFP | LIQGTSL | RTYPREK | TVSPRFL | YSGASTL |
| GHYISAN | LMPSYPR | SAAWNKS | TWSLDYP | YYNTTPN |

TABLE3
Peptide sequences identified from screening with cyclic
7-mer Phage Display Library, where all peptides are written
from N-terminus to C-terminus, have free a N-terminus and
an amidated C-terminus and are cyclized through a disulfide
bridge through two cysteine residues by a disulfide bond.
Cyclic 7-mer Peptide
Sequences

| | | | |
|---|---|---|---|
| CAGHNRDRC | CIAARHMNC | CSEGLLNTC | CSGWQVRMC |
| CAKSPMNCC | CILLPDKCL | CSEHNLQTC | CSHMEYPRC |
| CASKSTHDC | CKAALTRWC | CNMQITKGC | CSISSLTHC |
| CDAMIGKSC | CKDHVTRVC | CNPEHNNHC | CSNHRIMSC |
| CDFIMGITC | CKHLLGENC | CNPTHYRSC | CSQLPWYSC |
| CDGHDQSLC | CKLTTQMMC | CNQNASHYC | CSSPFPEFC |
| CDHPHKQQC | CKMSMLHNC | CNQTAARVC | CSSVTDRWC |
| CDHTYTNKC | CKSMMRLNC | CNRWHHLEC | CSTNSHSRC |
| CDKFHELQC | CKTLQPWTC | CNSFGVSMC | CSVGTNFQC |
| CDNIMTPVC | CLDIFSSSC | CNTGSPYEC | CTERTSTEC |
| CDQMWHTSC | CLHGDVAYC | CNTTEAASC | CTGKNAPKC |
| CDRTISNKC | CLKLGEKWC | CPFWLSGHC | CTILMKILC |
| CEDLTTLSC | CLKNQSDQC | CPRDLGTDC | CTKSLAHTC |
| CEGQRWMQC | CLNSSQPSC | CPVALSTKC | CTLRDSPHC |
| CELGTVQSC | CLRTSNPAC | CPVISNGSC | CTNANHYFC |
| CFGQGTLQC | CLVSQHTDC | CQGNPSLRC | CTNTNTAIC |
| CFNMFSRVC | CLWSTGATC | CQHLRGLLC | CTPGHTNRC |
| CGDGSQRTC | CMAPDSRVC | CQMQLRSAC | CTPSFSKIC |
| CGGGPLYMC | CMARYMSAC | CQNWISRFC | CTQMNDSFC |
| CGHSNLSNC | CMERMSLRC | CQPRNLNNC | CTQSSAMSC |
| CGLKALKEC | CMGFSNMSC | CQYETPRYC | CTVRTSADC |
| CGNSSLNRC | CMSTGLSSC | CRGATPMSC | CVGMQSNTC |
| CGYSSFNRC | CMSWSLQRC | CRINPMSNC | CVNLQKDMC |
| CHDLNGSMC | CNAKHHPRC | CRSANIYTC | CVPILEGTC |
| CHNEGNRAC | CNENIVHHC | CRSATHSAC | CVPMQDHTC |
| CHNRVPLMC | CNFLYSWTC | CRSQSGSNC | CVQMPAHSC |
| CHPVSGQKC | CNHDATHTC | CSDARSPKC | CYAFNYPHC |
| CHSDANSIC | CNIIHHQTC | CSGPGINLC | CYGNVTNTC |
| CHYNAHRTC | CYVSKNNSC | | |

TABLE4
Peptide sequences selected/identified from screening with
Bicyclic Phage Display Library A and B, where all peptides are
written from N-terminus to C-terminus, have a free N-terminus
and an amidated C-terminus and are cyclized through three
cysteine residues withTBMB (tribromomethylbenzene).
Bicyclic PeptideSequences

| | | | |
|---|---|---|---|
| CAAHQYCWTSC | CLTAHCPQSISC | CQLNMCTSANNC | CSYLCEPAQHVC |
| CAEQSCIFNLC | CLTQPCNNPRPC | CQLQLFCQTRTC | CTAPGNCSQLC |
| CALLIDCQYPLC | CLVPCTQYVC | CQPRHCIHSTVC | CTDSCPPQSC |
| CALSCHQVSLC | CMRACVMQFDC | CQPSTSCLIQRC | CTGNCVSSVGC |
| CAPLCGHRVPQC | CNDVTKLCSQFC | CQPTPTCGWTC | CTLEICRSQLGC |
| CARACQFGAC | CNLSCTSQTLEC | CQQFGQCSQFSC | CTLNCNSGFQRC |
| CDHQCGDHLC | CNLSPCLLPPQC | CQQRYSCFTNC | CTLTQCSLSKAC |
| CDQELCRELTSC | CNPPHICQNPKC | CQQYNCVPVGRC | CTPLCTPQHVC |
| CDQGDCHQKINC | CNQRPPFCLVRC | CQSFSCGQRLSC | CTQLCTASPFSC |
| CDVPCVAQYIC | CPAVLSCTAEQC | CQSLECAMRAHC | CTQQCPSSVC |
| CEKKYCTQQLPC | CPDQCQFSSC | CQSPSLCMGLPC | CTQVPCTPYQGC |
| CEMLQSCQQDWC | CPDTCQAAFFLC | CQSPWCQRLDLC | CTRDCPSQAHC |
| CETRGCYQRFRC | CPESCLDLQWC | CQSQDHCFHKDC | CTRVCSSSQLYC |
| CFKQNCSQSRSC | CPMALCSQGATC | CQTDVCQRTIC | CTSSLCQLSVLC |
| CFQLCPSVDFC | CPPQRRCTAFAC | CQYNDCDMLHC | CTTSCVKSSIC |
| CGAQGCFGVQSC | CPQLSCPSGGSC | CRAACNPFIC | CTVPTCSQSLRC |
| CGGGICRTHNC | CPQPQPCLRTSC | CREVTCHHLQC | CVGQEPCLSYTC |
| CGQVCNQKVC | CPQSSCQGLRLC | CRPQECAQHVC | CVKSCGQSVC |
| CHRQLCSPSEC | CPTMTVCQHPRC | CRQAYCSNLLLC | CVNSCSSLKC |
| CHRTPCSLPTTC | CPTSACMQQSGC | CRSETCAYQDC | CVNSLCTLPSQC |
| CIARDCWQGFSC | CPVPQCDPKKLC | CRSTPCQNQLEC | CVPSQWCYAQRC |
| CIEQPACPNIFC | CQAGVICLQQVC | CSHQCRSSELLC | CVPTCSRSGC |
| CIPIKRCNDQLC | CQAVCQLGPC | CSKQHTCVSPVC | CVQLTCEYLYAC |
| CISKCTSVAQSC | CQDGQCPRNC | CSLITQCGGVGC | CVWQGCALNWRC |
| CISLQQLCIRAC | CQDLCGQMVC | CSMGMCALPWQC | CVYTSCVQSLTC |
| CISRVGCQNPMC | CQFHIGCYSNC | CSNICLAKPHC | CWEQACSQEC |
| CLPSCQHAEIC | CQHPCKSTVPNC | CSQSNCVKAC | CWRSCPKGYC |
| CLRESACSKQC | CQISHCQNMIIC | CSSHVICNSNSC | CYAQRCGVTGC |
| CLRNCDYVQPPC | CQLINLCHDFLC | CSSREQCMITVC | CYESCRVQSALC |
| CLSQFCVIDC | CQLISCTGGLQC | CSTLDRCYQLC | CYHVRPCSSQLC |
| CLSSGCSAQDLC | CQLLCVQSSSEC | CSWYKCFNQPSC | CYMPCGQSVVC |
| CYQSPCPSGLC | CYNQRSSCAMSC | CYSGCGNLQGC | |

The present invention provides methods of treating bacterial infections, and related disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which bacterial infections may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a bacterial infection, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Therapeutics

Peptide antibiotics of the invention are useful for the treatment of bacterial infections (e.g., infections associated with Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Enterococcus, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, or Yersinia). In some embodiments the infection is a Staphylococcus or Enterococcus infection. In other embodiments, the infection is a B. subtilis, E. coli, E. faecalis or S. aureus infection. In other embodiments, the infection is methicillin-resistant S. aureus (MRSA), low level vancomycin resistant E. faecium, or high level vancomycin resistant E. faecium.

For therapeutic uses, the compositions or agents identified using the methods disclosed herein may be administered systemically, for example, formulated in a pharmaceutically-acceptable buffer such as physiological saline. Treatment may be accomplished directly, e.g., by treating the subject with peptides of the invention. Preferable routes of administration include, for example, subcutaneous, intravenous, interperitoneally, intramuscular, or intradermal injections which provide continuous, sustained levels of the drug in the patient. Treatment of human patients or other animals will be carried out using a therapeutically effective amount of an anti-pathogenic agent in a physiologically-acceptable carrier. Suitable carriers and their formulation are described, for example, in Remington's Pharmaceutical Sciences by E. W. Martin. The amount of the agent (e.g., a peptide antibiotic) to be administered varies depending upon the manner of administration, the age and body weight of the patient, and with the type of disease and extensiveness of the disease. Generally, amounts will be in the range of those used for other agents used in the treatment of other bacterial diseases, although in certain instances lower amounts will be needed because of the increased specificity of the compound. A compound is administered at a dosage that inhibits bacterial proliferation. For example, for systemic administration a compound is administered typically in the range of 0.1 ng-10 g/kg body weight.

The administration of a peptide for the treatment of an infection may be by any suitable means that results in a concentration of the therapeutic peptide that, combined with other components, is effective in ameliorating, reducing, or stabilizing a bacterial infection. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active peptide substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a bacterial infection. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

Phage display has proven to be a useful tool for identifying antimicrobial peptides by screening molecular targets, such as replication/cell division enzymes and host-pathogen virulence factors, or whole bacterial cells.¹³⁻¹⁴ Several reports detail the use of phage display for the isolation of peptides with antibacterial activity against different Gram-positive and Gram-negative bacteria.¹⁵⁻¹⁸ Mirror image phage display as a concept was pioneered by Kim and co-workers to select ‘D-peptides’ from screening experiments.¹⁹ Briefly, phage display peptide libraries are panned against the D-enantiomer of the target of interest. This results in the selection of L-peptide ligands that bind specifically to the D-target. By symmetry arguments, if the antipode of the ligand (D-peptide) is chemically synthesized, it will bind specifically to the native L-target with the same affinity. This technology has been successfully used to discover D-peptide inhibitors of HIV entry into host cells, the p53-MDM2 complex, VEGF, stabilization of amyloid beta (Aβ) peptide as well as D-peptide ligands for the cell-surface carbohydrates.²⁰⁻²⁴ Identifying D-peptide ligands for several different target molecules using mirror image phage display has gained popularity in the recent past. Martin and co-workers recently reported the identification of lipid II binding lipopeptide ligands with D-amino acids against vancomycin-resistant bacteria.²⁵

We describe here the discovery and characterization of peptide antibiotics composed of D-amino acids using mirror image phage display that are effective against S. aureus, MRSA and VRE that display high proteolytic and metabolic stability, and exhibit little to no toxicity. We accomplished this goal by screening 7-12 residue peptides (linear, cyclic and bicyclic) against enantiomers of (i) a derivative of the β-lactam antibiotic cephalosporin that mimics a high energy conformation of the D-Ala-D-Ala terminus of peptidoglycan structures in bacterial cell walls; and (ii) the cell wall crosslinking pentapeptide precursor. The new peptide scaffolds described here and variants thereof are useful as antibiotics.

### Example 1: Design and Synthesis of Target Molecules for Phage Display Screening

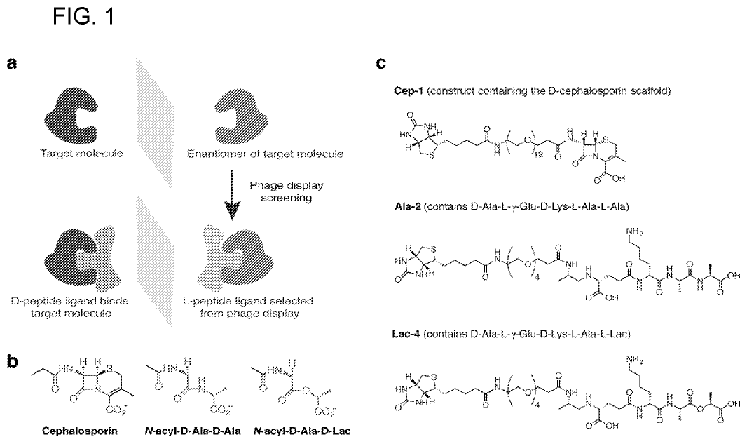

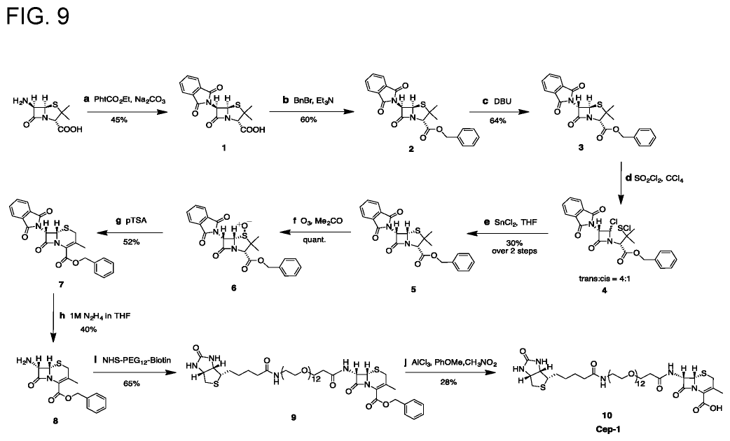

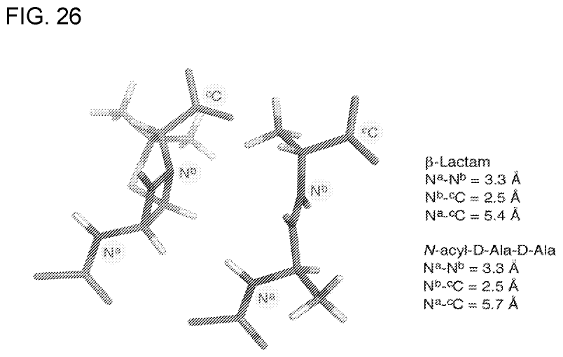

Vancomycin, the bedrock of antibiotics in the ICU, targets the final step of bacterial cell wall biosynthesis, and inhibits the transpeptidase crosslinking reaction by binding to the D-alanyl-D-alanine terminal end of the pentapeptide unit of the un-crosslinked peptidoglycan (PG). The vancomycin:D-Ala-D-Ala complex is held together by five hydrogen bonds with a reasonably tight binding affinity (K_{d}˜1 μM).²⁶ It is important that target molecules used for phage display screening maintain a desired conformation during affinity selection. Tipper and Strominger proposed that penicillin, a β-lactam antibiotic, mimics a high energy conformation of D-Ala-D-Ala, and thereby inhibits the cross-linking of bacterial cell walls by irreversibly binding the active site of the peptidoglycan transpeptidase enzyme (FIGS. 1 and 26).²⁷ We therefore hypothesized that using a β-lactam might enable selection of high-affinity binders. The enantiomer of cephalosporin was incorporated in the construct Cep-1 as a structurally rigid mimic of L-Ala-L-Ala (FIG. 9) using a slightly modified version of a previously reported synthetic approach starting from 6-aminopenicillanic acid.²⁸⁻²⁹ In addition, we also incorporated the enantiomer of the pentapeptide precursor of the bacterial cell wall (D-Ala-L-γ-Glu-D-Lys-L-Ala-L-Ala) into a short linear peptide target (Ala-2) which was chemically assembled using solid-phase peptide synthesis (as shown below) using t-Boc chemistry. Both target molecules were coupled to a polyethyleneglycol (PEG) linker, and a biotin moiety was attached to the N-terminus as a handle for immobilization on streptavidin beads during affinity selections. It is known that there is no significant difference in binding of vancomycin to UDP-MurNAc-L-Ala-D-γ-Glu-L-Lys-D-Ala-D-Ala versus to the pentapeptide precursor alone lacking the glycan.³⁰⁻³¹ The carbohydrate backbone that harbors the pentapeptide precursors during biosynthesis was therefore omitted from the target molecules. Another short peptide, incorporating α-Glu instead of γ-Glu in the peptide backbone (Ala-3) (shown below) was also prepared to interrogate how an extended backbone compound alters affinity selections.

### Example 2: Panning Against Vancomycin-Sensitive Bacteria

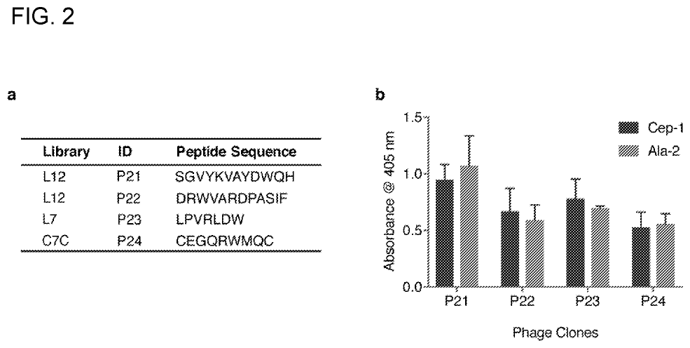

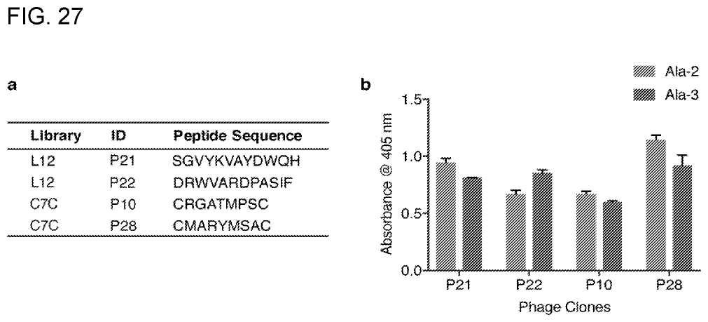

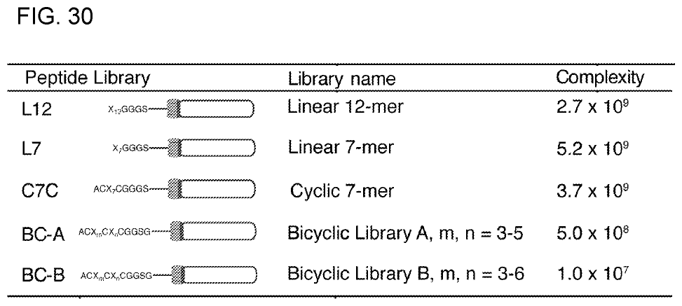

Phage display allowed us to access diverse peptide libraries composed of up to ˜10⁹ members (FIG. 30). Commercially available linear and cyclic phage display (Ph.D.) libraries, Ph.D.-12, Ph.D.-7 and Ph.D.-C7C, that display peptides with randomized residues fused to the pIII minor coat protein of M13 phage, were used in initial screenings. Affinity selections were performed using two different strategies, a high yield selection and a more stringent one. This results in identification of tight binders, as well as elevating the chance of finding molecules with moderate binding. In the stringent selections, 25 ng of the target molecules in each round were subjected to four rounds of biopanning, whereas 5 μg of the target molecules was used in the first round, and then decreased to 2 μg, 500 ng, and 100 ng in the successive rounds of high yield selections. After four subsequent rounds, 50 phage clones were randomly picked from each screening for DNA sequencing to identify the peptide sequences. These sequences revealed the short hydrophobic motifs, “TTL”, “I/L-S/T”, “Q/N-S/T”, “LQ”, “G-S/T/V”, “G-x-S/T”, “L-K/R”, “R-L/V”, “VLS”, “N/Q-K/R; and several consensus sequences were found. Four peptides with identical sequences were enriched in independent affinity selections using Cep-1 and Ala-2 in spite of their structural differences (FIG. 2), and slight differences in their binding affinities were observed as judged by phage-ELISA. Selection of identical sequences from these panning experiments is consistent with specific binding interactions between L-Ala-L-Ala and both target molecules Cep-1 and Ala-2. Additionally, peptides selected from screenings using Ala-2 and Ala-3 resulted in similar sequences albeit with different binding affinities in phage-ELISA (FIG. 27). Negative selections as controls were also included, and enrichment factors suggest that the selection was successful in favor of target-specific phage and not of background. While iterative affinity selections yielded several binders for each target molecule, the same short motifs were conserved in the case of both Ala-2 and Cep-1 further confirming that the screenings resulted in identification of peptide ligands that specifically bind to the target molecules. To determine candidates for further investigation, we used the results of a phage-ELISA assay for each phage clone, and peptides were chosen based on the amplitude of the ELISA signals as well as frequency of appearance. These select sequences were synthesized in D-enantiomeric form by solid-phase peptide synthesis (SPPS).

### Example 3: Bicyclic Peptide Ligands

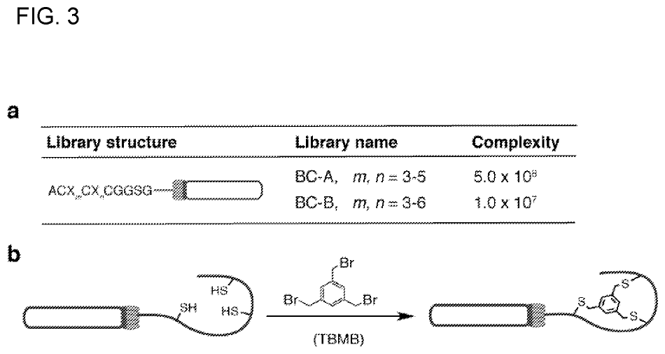

We also explored more constrained peptide ligands by using phage display employing bicyclic peptide libraries as described by Heinis and Winter (FIG. 3).³² These libraries are expressed as linear peptides containing three cysteine residues on the pIII minor coat protein of phage M13, and are later reacted with 1,3,5-tris(bromomethyl)benzene (TBMB) to obtain bicyclic scaffolds. Bicyclic peptides mimic the complementarity-determining regions of antibodies and are able to bind to their targets tighter than their linear or mono-cyclic counterparts.³³⁻³⁷ Bicyclic peptide libraries maintain diversity of members by incorporation of different loop lengths in the phage pool. Affinity selections were carried out using two bicyclic peptide libraries (libraries A and B) using the high yield strategy where 5 μg of target was used in the first round, and decreased to 2 μg in the second round. Phage titers (Tables 7-9) from the second round of selection were three orders of magnitude higher in number than the negative selection indicating that specific binders were present in the phage pool. Short peptide motifs similar to those obtained from commercial phage display libraries were observed from both target molecules, Cep-1 and Ala-2, namely “TTS”, “Q-R/K/L”, “SQL”, “VSQL”, “GQV”, “SV”, “SSV”, “T/P-Q”, “YQ-S/L”, “QRL” in addition to repeating bicyclic peptide sequences.

### Example 4: Antimicrobial Activity of D-Peptides

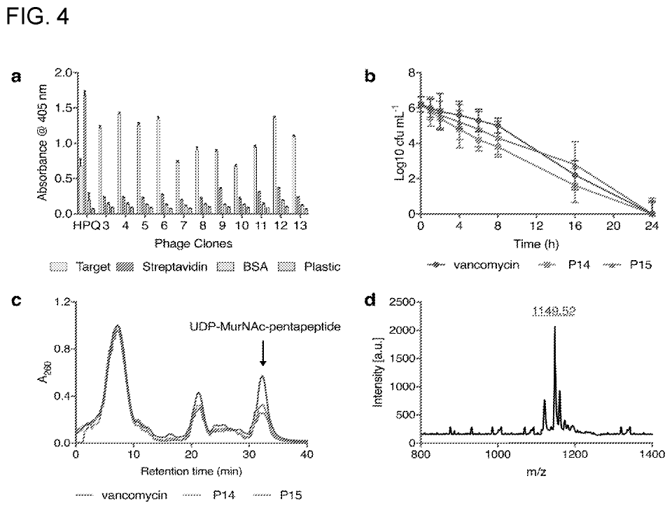

The L-peptides identified from five different phage display libraries were catalogued as described previously and selected sequences were synthesized in D-enantiomeric form by SPPS for further investigation in several biological assays. Peptides were assembled as C-terminal amides to mimic peptides displayed on the coat protein of phage particles. Antibiotic susceptibility of several Gram-positive strains (with one Gram-negative strain as control) was tested in presence of the synthetic D-peptides, and assessed according to a broth micro-dilution protocol described by National Committee for Clinical Laboratory Standards (NCCLS) with slight modifications specific for peptide antibiotics as described by Hancock.³⁸ Each test was conducted in triplicate with vancomycin and melittin serving as control peptides. Table 5 only lists the antibacterial activity of constructs having a minimal inhibitory concentration (MIC) value of <256 g/ml among the 170 D-peptides that were identified through screening of five different phage display libraries. Some of the D-peptides (linear 12-mer, cyclic 7-mer, and bicyclic) showed higher activity with MIC values in the 8 to 32 μg/ml range, while others had moderate activity with MIC values in the 64 to 128 μg/ml regime, and several showed no antibacterial activity (MIC value of >256 μg/ml). Positive antibiotic action was only observed against Gram-positive strains as expected (Table 5). The most active D-peptide, a bicyclic construct P14, displayed promising antibacterial action against S. aureus and MRSA with MIC values of 8 μg/ml and 32 μg/ml, respectively. In addition, it was also active against two vancomycin-resistant Enterococci strains, vanB (MIC=32 μg/ml) and vanA (MIC=128 m/ml); in contrast to vancomycin that has no activity against vanA. Another potent bicyclic structure P15 showed similarly high activity as P14 for vancomycin-sensitive bacterial strains, while it had moderate effect on the low-level vancomycin-resistant strain and no activity on the high-level one. Other phage display derived D-peptides showed MICs ranging from 8 μg/ml to 128 μg/ml against S. aureus and MRSA but not for vancomycin-resistant strains. The phage-ELISA results of selected active peptides are shown in FIG. 4a compared to a control peptide that contains the streptavidin specific binding motif “HPQ”. None of the four identical sequences identified from both Cep-1 and Ala-2 screens showed antibacterial activity.

TABLE 5
Antibiotic activity of D-peptides against vancomycin-sensitive bacteria

| | | | | MIC (μg/ml) | | | | | |
|---|---|---|---|---|---|---|---|---|---|
| ID | Lib | Sequence | E. coli^{a} | B. subtilis^{b} | S. aureus^{c} | MRSA^{d} | E. faecalis^{e} | VRE^{f} | VRE^{g} |
| P1 | | Vancomycin | >256 | 0.5 | 0.5 | 0.5 | 1 | 32 | >256 |
| P2 | | Melettin | 32 | 2 | 2 | 4 | 8 | 32 | 32 |
| P3 | L12 | VGSNLRLLHQWK | >256 | 32 | 32 | 64 | 32 | >256 | >256 |
| P4 | L12 | VHWDFRQWWQPS | >256 | 32 | 32 | 64 | 32 | 128 | >256 |
| P5 | L7 | KCCYTLP | >256 | 128 | 128 | >256 | >256 | >256 | >256 |
| P6 | L7 | VLTRCCG | >256 | 128 | 128 | >256 | >256 | >256 | >256 |
| P7 | L7 | TGFLVNV | >256 | 32 | 64 | >256 | >256 | >256 | >256 |
| P8 | L7 | HYIDFRW | >256 | 32 | 62 | 64 | 64 | >256 | >256 |
| P9 | C7C | CLKLGEKWC | >256 | 32 | 64 | 64 | 64 | >256 | >256 |
| P10 | C7C | CRGATPMSC | >256 | 128 | 128 | 128 | 256 | >256 | >256 |
| P11 | C7C | CSISSLTHC | >256 | 32 | 32 | 64 | 32 | 64 | >256 |
| P12 | C7C | CLWSTGATC | >256 | 64 | 64 | 64 | 128 | >256 | >256 |
| P13 | C7C | CDNIMTPVC | >256 | 64 | 64 | 64 | 128 | >256 | >256 |
| P14 | BC-A | CQTDVCQRTIC | >256 | 8 | 8 | 32 | 32 | 32 | 128 |
| P15 | BC-B | CSLITQCGGVGC | >256 | 8 | 8 | 32 | 16 | 128 | >256 |

Lib = phage display peptide library
MIC = minimum inhibitory concentration
^{a}E. coli ATCC 25992
^{b}B. subtilis ATCC 6633
^{c}S. aureus ATCC 6538
^{d}Methicillin-resistant S. aureus (MRSA) ATCC 43300
^{e}E. faecalis ATCC 29121
^{f}low level vancomycin resistant E. faecium (vanB) ATCC 51299
^{g}high level vancomycin resistant E. faecium (vanA) ATCC 51559
The bactericidal activity of an antimicrobial agent against a particular organism is related to its mechanism of action.³⁹ Briefly, agents that disrupt the cell wall or cell membrane, or interfere with essential bacterial enzymes, are likely to be bactericidal, whereas those agents that inhibit ribosome function and protein synthesis tend to be bacteriostatic. The bacteria killing kinetics of P14 and P15 was interrogated by a “time-kill” assay and compared to vancomycin to assess the mode of action. These “time-kill” assays showed time-dependent reductions in the number of colony-forming units per mL (cfu/ml) by P14 and P15. Completely killing of bacterial cells was observed in 24 h, similar to vancomycin. While both macrocyclic peptides exhibited similar killing kinetics, peptide P14 exhibited a modestly faster rate than vancomycin against S. aureus (Fig. 4b).

### Example 5: Mechanism of Action of Potent Bicyclic Peptides

We next examined the effect of P14 and P15 on cell wall biosynthesis in live bacteria. Blockade of this biosynthetic pathway causes accumulation of the water soluble intracellular intermediate lipid II precursor UDP-MurNAc-L-Ala-D-γ-Glu-L-Lys-D-Ala-D-Ala.⁴⁰ This intermediate is detectable after extraction from the cytosol in response to antibiotic administration by chromatographic methods.

Incubation of S. aureus with P14 and P15 at 10-fold MIC led to accumulation of UDP-MurNAc-pentapeptide at levels similar to vancomycin as has been previously reported (FIGS. 4c and 4d). This result suggests that the action mechanism of the two bicyclic constructs is similar to vancomycin and is the outcome of cell wall biosynthesis inhibition.

### Example 6: Phage Selection Against Vancomycin-Resistant Strains

Since the first report of vancomycin-resistant Staphylococcus aureus (VRSA) in the US in 2002, the threat of vancomycin being rendered ineffective has become real. More concerning is the possibility that vancomycin resistance may spread to multi-drug resistant pathogenic bacteria, such as methicillin resistant S. aureus (MRSA). Bacteria exhibiting vancomycin resistance have a modified cell wall precursor pentapeptide, where some of the terminal D-alanine residues are substituted with D-lactate. This substitution results in an estimated 1000-fold reduced binding affinity for vancomycin due to the loss of one hydrogen bond and introduction of a repulsive interaction.⁴¹ Several approaches have been tried to overcome vancomycin-resistance including modification of vancomycin itself, utilizing combinatorial libraries to identify binders of the D-Ala-D-Lac precursor, and the design of vancomycin-inspired semi-synthetic glycopeptide derivatives and increasing affinity by use of dimeric vancomycin.⁴²⁻⁶⁰

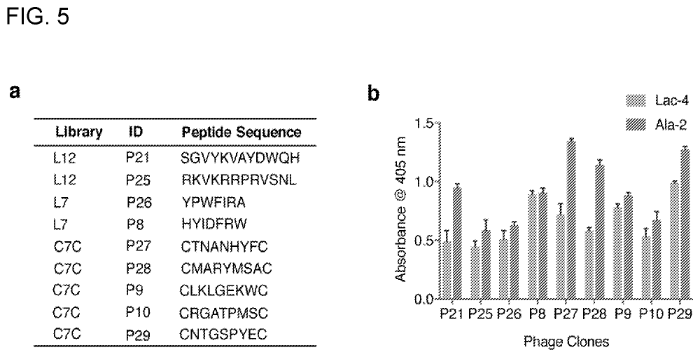

We synthesized the enantiomer of the pentapeptide precursor of vancomycin-resistant bacterial cell walls (D-Ala-L-γ-Glu-D-Lys-L-Ala-L-Lac) with a PEG linker and a biotin group to use as the target molecule in phage screening (Lac-4, as shown below). Both commercial phage display and bicyclic libraries were used in the selections. All commercial phage libraries yielded sequences with short hydrophobic peptide motifs, “TTL”, “I/L-S/T”, “Q/N-S/T”, “LQ”, “GQS”, “G-S/V”, “G-X-S”, “L-K/R”, “RV”, “VLS”, “Q/N-K/R” similar to previous screenings with target molecules Cep-1 and Ala-2. Target molecules Ala-2 and Lac-4 resulted in selection of several identical peptide sequences with different binding affinities by phage-ELISA (FIG. 5). Since short pentapeptide precursors for vancomycin-sensitive and resistant strains differ only in a single residue (Ala to Lac), selection of similar peptide sequences was to be expected. It also suggests that these peptides might have binding interactions with entire pentapeptide precursor rather than solely with the D-Ala-D-Ala terminal end.

In addition, screening of Lac-4 against bicyclic peptide libraries resulted in short hydrophobic peptide motifs, “GQV”, “QL”, “SSV”, “T/P-Q”, “YQ-S/L”, “QRL” as previously observed in Cep-1 and Ala-2. These observations are in the agreement with the findings of Liskamp and co-workers where ligands binding to D-Ala-D-Lac had a significant number of polar amino acids, mostly glutamine and serine residues.⁴⁴ None of the D-peptides identified from screenings of five different peptide libraries showed significantly high antibacterial activity for vancomycin-resistant bacteria, whereas one D-peptide, P18, had moderate activity for the low-level vancomycin-resistant bacterium (Table 6). On the other hand, six D-peptides had moderate activity for S. aureus, MRSA, and E. faecalis strains with MIC values ranging from 32 to 128 μg/ml.

TABLE6
Antibiotic activity of D-peptides against
vancomycin-resistant bacteria

| | | | MIC(μg/ml) | | | | | | |
|---|---|---|---|---|---|---|---|---|---|
| | | | E. | B. | S. | | E. | | |
| ID | Lib | Sequence | coli^{a} | subtilis^{b} | aureus^{c} | MRSA^{d} | faecalis^{e} | VRE^{f} | VRE^{g} |
| P1 | | Vancomycin | >256 | 0.5 | 0.5 | 0.5 | 1 | 32 | >256 |
| P2 | | Melittin | 32 | 2 | 2 | 4 | 8 | 32 | 32 |
| P8 | L7 | HYIDFRW | >256 | 32 | 32 | 64 | 64 | >256 | >256 |
| P9 | C70 | CLKLGEKWC | >256 | 32 | 64 | 64 | 64 | >256 | >256 |
| P10 | C7C | CRGATPMSC | >256 | 128 | 128 | 128 | 256 | >256 | >256 |
| P16 | BC-A | COYNDCDMLHC | >256 | 64 | 64 | 128 | 128 | >256 | >256 |
| P17 | BC-A | CLSQFCVIDC | >256 | 128 | 128 | >256 | >256 | >256 | >256 |
| P18 | BC-A | CGGGICRTHNC | >256 | 16 | 16 | 32 | 128 | 128 | >256 |
| P19 | BC-B | CTRVCSSSQLYC | >256 | 128 | 128 | >256 | >256 | >256 | >256 |
| P20 | BC-B | CGAQGCFGVQSC | >256 | 32 | 32 | 256 | 256 | 256 | >256 |

Lib = phage display peptide library
MIC= minimum inhibitory concentration
^{a}E. Coli ATCC25992
^{b}B. subtilis ATCC6633
^{c}S. aureus ATCC6538
^{d}Methicillin-resistant S. aureus (MRSA) ATCC43300
^{e}E. faecalis ATCC29121
^{f}low level vancomycin resistant E. faecium (vanB) ATCC51299
ghigh level vancomycin resistant E. faecium (vanA) ATCC51559

### Example 7: D-Peptides Discovered in this Study are not Toxic

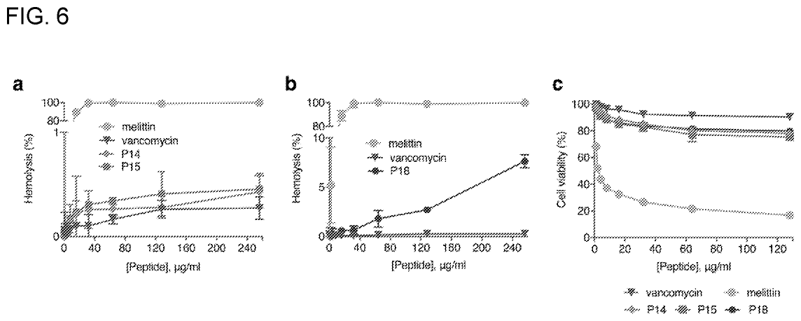

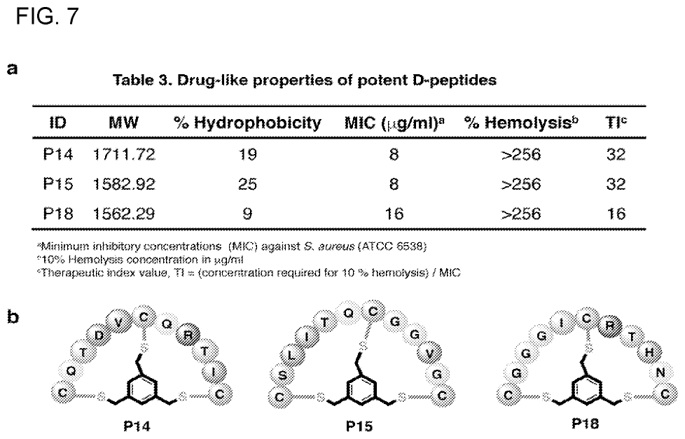

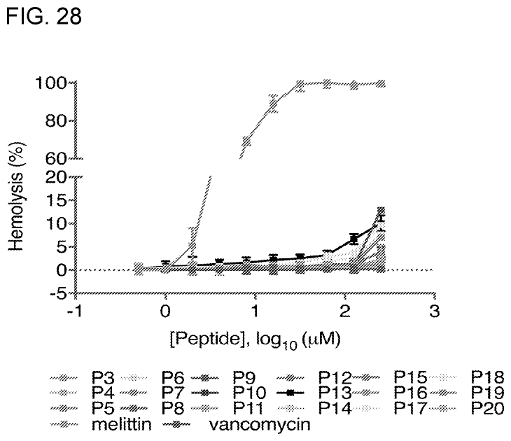

Toxicity is a major problem in the development of new antibacterials that target the bacterial cell wall.⁶¹ The lytic activity of D-peptides on human red blood cells (hRBC) was evaluated by a hemolysis assay along with melittin and vancomycin as positive and negative controls respectively. Only minimal hemolytic activity (less than 10%) was observed with D-peptides even at high concentrations (FIGS. 6a, 6b, and 28) as compared to the natural antimicrobial peptide, melittin. The nontoxic nature of the two most active D-peptides, P14 and P15, was also confirmed on mammalian (HeLa) cells as judged by using a colorimetric MTT assay. While many natural and designed antimicrobial peptides are cytotoxic for mammalian cells, the bicyclic D-peptides described here did not exhibit any toxicity on HeLa cells even at high concentrations and were comparable to observations with vancomycin (FIG. 6c). To catalog relative activities of D-peptides against bacteria and erythrocytes, therapeutic index (TI) values were calculated. Higher TI numbers indicate that the select D-peptides were more active against bacteria relative to their cytotoxic action on erythrocytes (FIG. 7).

### Example 8: Stability of Potent D-Peptides

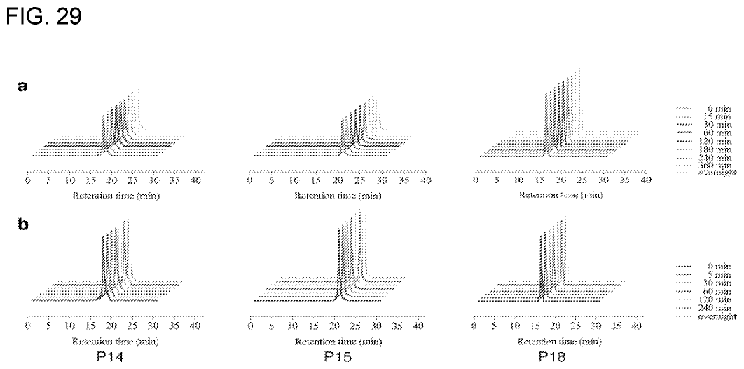

Peptides suffer from low metabolic stability as they are rapidly degraded by hydrolytic action of proteases and therefore are typically not orally bioavailable. Our approach to this problem obviates the use of L-amino acids, with the expectation that D-peptides would be refractory to protease action. We tested stability of three potent bicyclic peptides in human serum and pancreatin (simulated intestinal fluid containing digestive enzymes) by incubating 1 mg/ml of peptides separately in human blood and pancreatin solution for 24 h. RP-HPLC was employed to monitor the level of each active D-peptide remaining at various time points (FIG. 29). None of these peptides were digested even after a 24 h incubation. Pancreatin is a proteolytic mixture of digestive enzymes including amylases, lipases, and proteases (e.g. trypsin); we reasoned that degradation resistance to this protease cocktail would strengthen our working hypothesis that results in more stable peptide therapeutics. D-amino acid oxidase is the only known mammalian enzyme that metabolizes D-peptides in the kidney.⁶² Despite the refractory nature of our constructs towards degradation by pancreatin, detailed pharmacological studies will be required to determine whether or not gastrointestinal administration in preclinical models or ultimately, oral use in patients is a viable possibility. Bicyclic peptide scaffolds composed of D-amino acids proffer orally available and metabolically stable molecules that can be used as antibiotics. For every class of antibiotic introduced into human clinical use, it has been found that resistant organisms are selected for, and eventually become abundant in human infections. The need therefore remains to create platforms that can effectively generate new lead compounds that can then be subjected to traditional medicinal chemistry approaches to yield molecular entities that are effective as therapeutics in practice. S. aureus is especially notorious for its ability to develop resistance to antibiotics, and therefore novel strategies are urgently needed to tackle this global public health threat. Here, we report the discovery of peptide antibiotics composed of D-amino acids active against S. aureus, Methicillin-resistant S. aureus and vancomycin-resistant Enterococci with excellent antibacterial activity with MICs in the range of 8 μg/ml to 32 μg/ml. In addition, these compounds show minimal to no toxicity to both mammalian and red blood cells. Short peptides with high metabolic stability that target bacterial cell wall precursors with higher or equal affinities than vancomycin in drug sensitive and resistant bacterial strains are exciting and promising platforms for the development of new therapeutics. Initially, we investigated peptide antibiotics active against vancomycin-sensitive bacteria using five different peptide libraries (linear, monocyclic and bicyclic). Target design for affinity selections of vancomycin-sensitive bacteria was based on the enantiomer of the pentapeptide precursor of bacterial cell wall of S. aureus; a conformationally locked construct, Cep-1, and a flexible structure construct, Ala-2. Biopanning of peptide libraries resulted in identification more than a hundred L-peptide ligands that specifically bind to target molecules with different affinities as judged by phage-ELISA experiments compared to streptavidin binding peptide containing an “HPQ” motif. The peptides identified from these screens (7-12 mer peptides) shared short hydrophobic motifs that were similar. Peptides were then synthesized in D-enantiomeric form by SPPS and evaluated for antibacterial activities against several strains. The most potent peptides identified (P14 and P15) during affinity selections against vancomycin-sensitive bacteria using Cep-1 and Ala-2 as targets, were from phage display of bicyclic libraries. Bicyclic peptide P14 showed high antibacterial activity not only against S. aureus, MRSA and Enterococci but also against two vancomycin-resistant Enterococci strains vanB and vanA. The other bicyclic peptide P15 had similar inhibitory activity on vancomycin-sensitive bacteria whereas it was less active against vancomycin-resistant strains. The minimum inhibitory concentrations (MIC) values of these un-optimized antibacterials from target-based screenings are in the 16-32 μg/ml have been deemed sufficient for primary hit compounds.⁶³ We envision that by modifications guided by computational efforts and by use of noncanonical side chains in the scaffolds, the activities and pharmacological properties can be rapidly improved.⁶⁴

We subsequently employed this strategy to target vancomycin-resistant bacteria by using Lac-4 as the bait molecule in phage display screenings. Among the identified peptides, bicyclic peptide P18 showed excellent activity against vancomycin-sensitive bacteria and moderate activity against the vancomycin-resistant strain, vanB. None of identified peptides displayed high activity against both vanB and vanA type vancomycin-resistant Enterococci. The three most potent bicyclic peptides, P14, P15 and P18, neither caused lysis of erythrocytes nor showed significant toxicity against mammalian cells (HeLa) at concentrations up to 256 μg/ml, suggesting that they are promising candidates for further elaboration. Since peptide antibiotics identified here composed of D-amino acids, all of them showed high stability in human serum and also in the presence of protease cocktail pancreatin.

### Example 9: Materials and Methods

### Synthesis of Construct Cep-1 Containing an Enantiomeric Cephalosporin Scaffold

Construct Cep-1 containing an enantiomeric cephalosporin scaffold was synthesized using the procedure outlined in FIG. 9.

### Synthesis of 2S,5R,6R)-6-(1,3-dioxoisoindolin-2-yl)-3,3-dimethyl-7-oxo-4-thia-1-azabicyclo[3.2.0]heptane-2-carboxylic Acid (1)

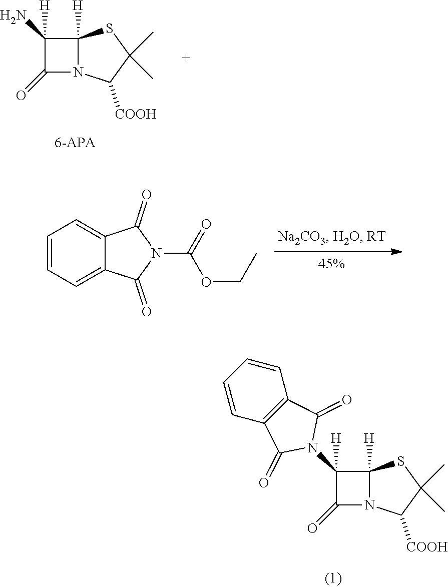 [SMILES: CC1(C)S[C@@H]2[C@H](N)C(=O)N2[C@H]1C(=O)O ; CCOC(=O)N1C(=O)c2ccccc2C1=O ; CC1(C)S[C@@H]2[C@H](N3C(=O)c4ccccc4C3=O)C(=O)N2[C@H]1C(=O)O]

To a vigorously stirred solution of 6-aminopenicillanic acid (3.00 g, 13.9 mmol) and Na₂CO₃ (1.47 g, 13.9 mmol) in water (20 ml) was added N-carboethoxyphthalimide (3.05 g, 13.9 mmol). The mixture was stirred at room temperature for 2 h and then washed with CH₂Cl₂ (3×5 ml). The aqueous layer was mixed with a fresh portion of CH₂Cl₂ (20 ml) and acidified during vigorous stirring with 1 M HCl (40 ml). The phases were separated and the extraction was completed with two additional portions of CH₂Cl₂ (2×5 ml). The combined organic extracts were washed with water (2×10 ml) and satd. brine (5 ml). The organic layer was dried over MgSO₄, and evaporated in vacuo to afford compound 1 (2.30 g, 45%) as an off-white solid which was used in the next step without further purification. ¹H NMR (CDCl₃, 500 MHz) δ 7.89 (dddd, 2H, J=8.5, 5.5, 3.0, 1.0 Hz), 7.77 (dddd, 2H, J=8.5, 5.5, 3.0, 1.0 Hz), 5.69 (dd, 1H, J=4.0, 1.0 Hz), 5.59 (dd, 1H, J=4.0, 1.0 Hz), 4.71 (s, 1H), 1.85 (s, 3H), 1.62 (s, 3H).

### Synthesis of (2S,5R,6R)-benzyl-6-(1,3-dioxoisoindolin-2-yl)-3,3dimethyl-7-oxo-4-thia-1-azabicyclo[3.2.0]heptane-2-carboxylate (2)

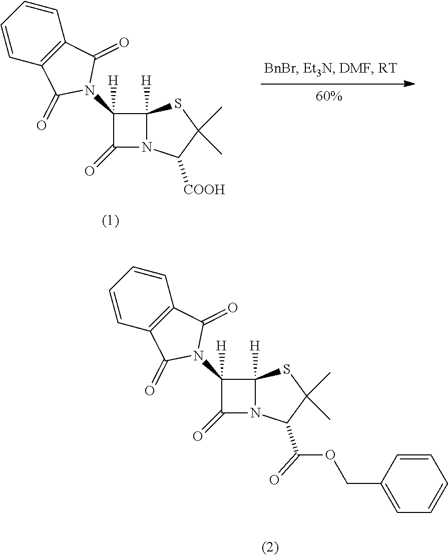 [SMILES: CC1(C)S[C@@H]2[C@H](N3C(=O)c4ccccc4C3=O)C(=O)N2[C@H]1C(=O)O ; CC1(C)S[C@@H]2[C@H](N3C(=O)c4ccccc4C3=O)C(=O)N2[C@H]1C(=O)OCc1ccccc1]

To a stirred solution of compound 1 (2.3 g, 76.64 mmol) in DMF (12 ml) was added triethylamine (0.93 ml, 6.64 mmol) followed by benzyl bromide (0.99 ml, 8.3 mmol). The mixture was stirred at room temperature for 6 h and then poured into vigorously stirred ice water (55 ml). The resulting suspension was extracted with CHCl₃ (3×25 ml), organic layers were combined and washed with satd. NaHCO₃ (3×15 ml), water (3×15 ml), and saturated brine (15 ml). The organic phase was dried over MgSO₄ and evaporated in vacuo to obtain the crude compound as light yellow oil. The compound was purified by flash chromatography on silica (60% hexanes/EtOAc) to afford compound 2 (1.87 g, 60%) as a white solid. ¹H NMR (CDCl₃, 300 MHz) δ 7.88 (dd, 2H, J=5.4, 3.0 Hz), 7.77 (dd, 2H, J=5.4, 3.0 Hz), 7.42-7.37 (m, 5H), 5.68 (d, 1H, J=3.9 Hz), 5.59 (d, 1H, J=3.9 Hz), 4.70 (s, 1H), 4.51 (s, 2H), 1.80 (s, 3H), 1.44 (s, 3H).

### Synthesis of 2S,5R,6S)-benzyl-6-(1,3-dioxoisoindolin-2-yl)-3,3-dimethyl-7-oxo-4-thia-1-azabicyclo[3.2.0] heptane-2-carboxylate (3)

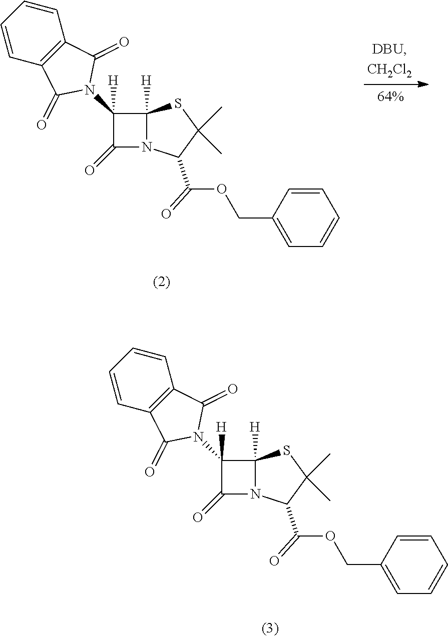 [SMILES: CC1(C)S[C@@H]2[C@H](N3C(=O)c4ccccc4C3=O)C(=O)N2[C@H]1C(=O)OCc1ccccc1 ; CC1(C)S[C@@H]2[C@@H](N3C(=O)c4ccccc4C3=O)C(=O)N2[C@H]1C(=O)OCc1ccccc1]

To a solution of ester 2 (1.87 g, 4.28 mmol) in CH₂Cl₂ (16 ml) was added 1,8-diazabicyclo[5.4.0]undec-7-ene (DBU) (0.042 ml, 0.28 mmol) and the mixture was stirred at room temperature for 90 min. The solution was washed with 1 M NH₄Cl (2×10 ml), water (3×10 ml), and satd. brine (5 ml). The organic phase was dried over MgSO₄ and evaporated in vacuo to afford a white foam. It was purified by flash chromatography on silica (60% hexanes/EtOAc) to afford compound 3 (1.87 g, 64%) as a white foam. ¹H NMR (CDCl₃, 500 MHz) δ 7.90 (dd, 2H, J=5.5, 3.0 Hz), 7.77 (dd, 2H, J=5.5, 3.0 Hz), 7.41-7.34 (m, 5H), 5.57 (d, 1H, J=2.0 Hz), 5.40 (d, 1H, J=2.0 Hz), 5.22 (d, 2H, J=2.0 Hz), 4.66 (s, 1H), 1.64 (s, 3H), 1.43 (s, 3H).

### (2S,5S,6S)-benzyl-6-(1,3-dioxoisoindolin-2-yl)-3,dimethyl-7-oxo-4-thia-1-azabicyclo[3.2.0]heptane-2-carboxylate (5)

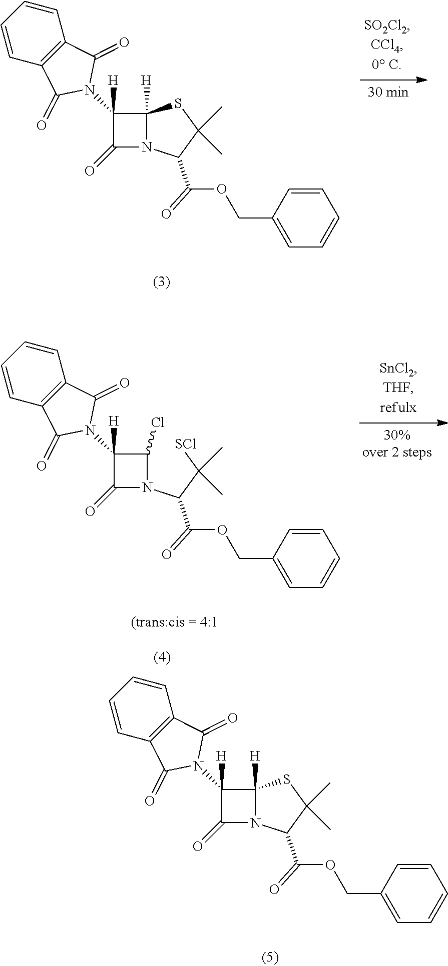

A solution of compound 3 (0.050 g, 0.114 mmol) in CCl₄ (2 ml) was treated with equimolar amount of sulfuryl chloride (0.114 mmol, 1 M solution in CCl₄) by stirring at 0° C. for 30 min. The solvents were evaporated in vacuo to give a mixture of the trans ds, cis ds (desired) and SM in a ratio of ca. 3:1:1.2 as a yellow foam. The foam was dissolved in THF (2 ml) and treated with anhydrous SnCl₂ (0.0167 g, 0.088 mmol). The mixture was stirred at room temperature for 2 h and then the solvent was evaporated in vacuo. The residual oil was dissolved in EtOAc (5 ml) and washed with water (3×5 ml) and satd. brine (5 ml). The organic phase was dried over MgSO₄ and evaporated in vacuo to give a white foam. It was purified by flash chromatography on SiO₂ (100% CH₂Cl₂-94% CH₂Cl₂/EtOAc) to afford compound 5 (0.005 g, 30% over two steps) as a white foam. ¹H NMR (CDCl₃, 300 MHz) δ 7.88 (dd, 2H, J=5.4, 3.0 Hz), 7.75 (dd, 2H, J=5.4, 3.0 Hz), 7.48-7.34 (m, 5H), 5.62 (d, 1H, J=3.9 Hz), 5.31 (d, 2H, J=6.0 Hz), 5.26 (d, 2H, J=3.9 Hz), 4.02 (s, 1H), 1.70 (s, 3H), 1.69 (s, 3H).

### Benzyl(2S,4S,5S,6S)-3,3-dimethyl-7-oxo-6-phthalimido-4-thia-1-azabicyclo[3.2.0]heptane-2-carboxylate 4-oxide (6)

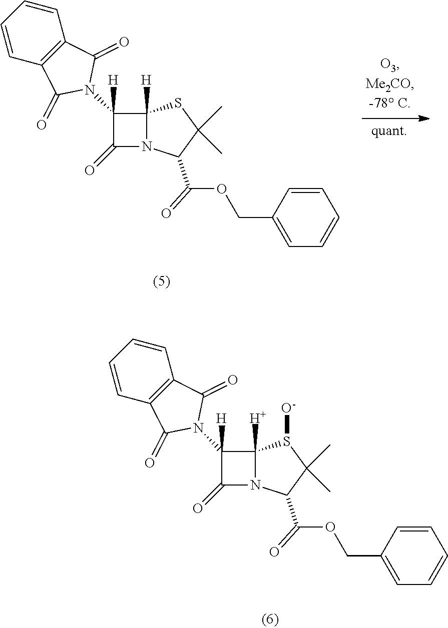

Compound 5 (0.115 g, 0.263 mmol) was dissolved in acetone (5.0 ml), cooled to −78° C., and treated with a stream of ozone. The solvent was evaporated in vacuo to give a white foam compound 6 (0.132 mg, quant. yield) that was used without purification. ¹H NMR (CDCl₃, 500 MHz) δ 7.91 (dd, 2H, J=5.5, 3.0 Hz), 7.80 (dd, 2H, J=5.5, 3.0 Hz), 7.48-7.35 (m, 5H), 5.92 (d, 1H, J=4.8 Hz), 5.37 and 5.30 (ABq, 2×1 H, J=12.0 Hz), 4.56 (d, 1H, J=4.8 Hz), 4.30 (s, 1H), 1.61 (s, 3H), 1.55 (s, 3H).

### (6S,7S)-benzyl-7-(1,3-dioxoisoindolin-2-yl)-3-methyl-8-oxo-5-thia-1-azabicyclo[4.2.0]oct-2-ene-2-carboxylate (7)

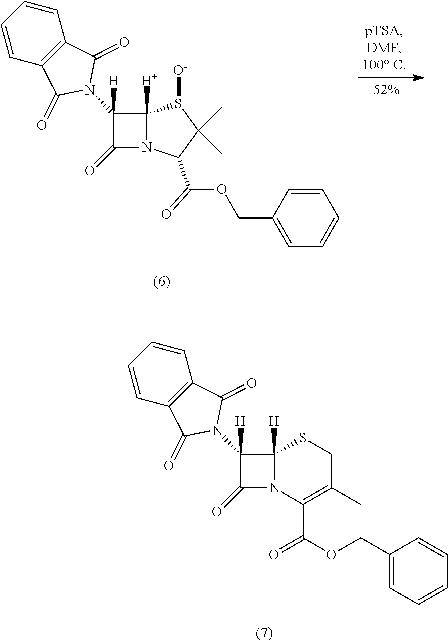

A stirred solution of sulfoxide compound 6 (0.132 g, 0.292 mmol) and anhydrous p-toluenesulfonic acid (pTSA, 0.0068 g, 0.0394 mmol) in THF (15 ml) was heated at 100° C. for 2 h. The reaction mixture was cooled to room temperature, diluted with EtOAc (5 ml) and washed with water (4×5 ml), satd. NaHCO₃ (3×5 ml), and satd. brine (2 ml). The organic layer was dried over MgSO₄ and evaporated in vacuo to yield a brown foam which was subjected to purification using flash chromatography (100% CH₂Cl₂ to 98% CH₂Cl₂/EtOAc) to afford compound 7 (0.059 g, 52%) as a white foam. ¹H NMR (CDCl₃, 300 MHz) δ 7.89 (dd, 2H, J=5.5, 3.0 Hz), 7.78 (dd, 2H, J=5.5, 3.0 Hz), 7.46-7.32 (m, 5H), 5.73 (d, 1H, J=4.3 Hz), 5.31-5.21 (m, 2H), 5.11 (d, 1H, J=4.3 Hz), 3.73 and 3.01 (q, 2×1 H, J=15.5 Hz), 2.33 (s, 3H).

### (6S,7S)-benzyl-7-amino-3-methyl-8-oxo-5-thia-1-azabicyclo [4.2.0]oct-2-ene-2-carboxylate (8)

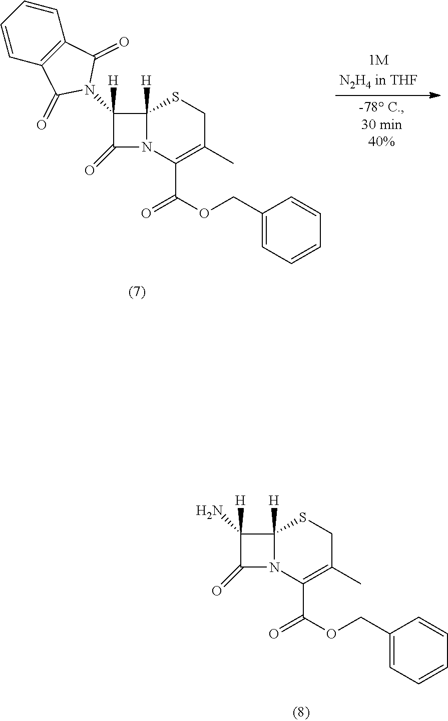 [SMILES: CC1=C(C(=O)OCc2ccccc2)N2C(=O)[C@H](N3C(=O)c4ccccc4C3=O)[C@@H]2SC1 ; CC1=C(C(=O)OCc2ccccc2)N2C(=O)[C@H](N)[C@@H]2SC1]

To a solution of compound 7 (0.0478 g, 0.110 mmol) at −78° C. in THF (2.0 ml) was added 1 M N₂H₄ solution in THF (0.220 ml, 0.220 mmol). The reaction mixture stirred at for 30 min without cold bath. The phthalhydrazide complex was decomposed by addition of 1 M HCl (0.28 ml) and the solvents were evaporated in vacuo. The residual oil was dissolved, with vigorous stirring, in water (2.0 ml) and the insoluble phthalhydrazide was removed by filtration. The aqueous filtrate was layered with EtOAc (2.0 ml) and basified with satd. NaHCO₃ to pH 8. The two phase mixture was separated, and extracted with EtOAc (2×2 ml). The combined organic extracts were washed with water (3×2 ml) and satd. brine (1 ml), dried over MgSO₄, and evaporated in vacuo to give a thick yellow oil which was purified by flash chromatography (100% CH₂Cl₂ to 70% CH₂Cl₂/EtOAc) to afford compound 8 (0.021 g, 40%) as a white foam. ¹H NMR (CDCl₃, 500 MHz) δ 7.43-7.30 (m, 5H), 5.27 (d, 2H, J=4.1 Hz), 4.91 (d, 1H, J=4.9 Hz), 4.70 (d, 1H, J=4.9 Hz), 3.52 and 3.21 (ABq, 2×1 H, J=18.4 Hz), 2.11 (s, 3H).

### (6S,7S)-benzyl-7-(poly(ethylene glycol)ether-2-(biotinylamino)ethane)-3-methyl-8-oxo-5-thia-1-azabicyclo [4.2.0]oct-2-ene-2-carboxylate (9)

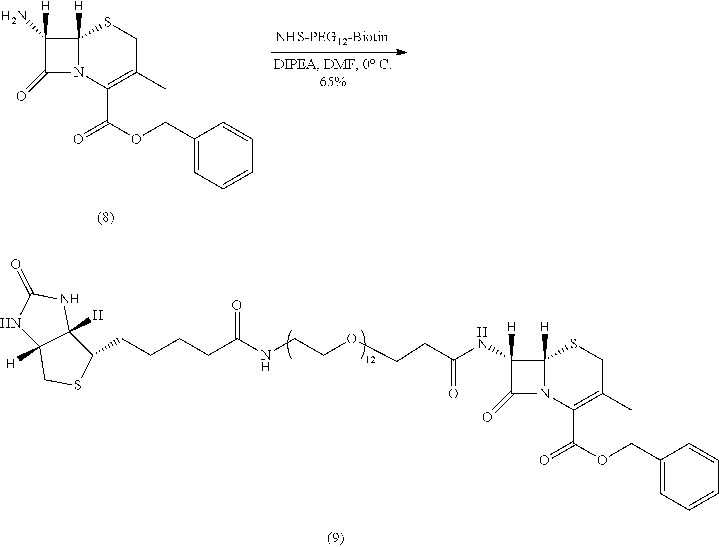

To a solution of compound 8 (0.007 g, 0.023 mmol) and NHS-PEG₁₂biotin (0.025 g, 0.026 mmol) at 0° C. in DMF (0.5 ml) was added DIPEA (12 ml, 0.069 mmol). The reaction mixture was stirred at 0° C. for 24 h and the solvents were evaporated in vacuo to give a thick yellow oil which was purified four times by RP-HPLC (rt=16.5 min, C18 semi-prep column, 30-40% B; A=98% H₂O/Acetonitrile; B=99% Acetonitrile/H₂O) to afford compound 9 as a white solid. ¹H NMR (CDCl₃, 500 MHz) δ 7.43-7.35 (m, 5H), 6.72 (bs, 1H), 5.81 (q, 1H, J=4.5 Hz), 5.28 (dd, 2H, J=20.0, 12.5 Hz), 4.98 (d, 1H, J=5.0 Hz), 4.55-4.50 (m, 1H), 4.32-4.36 (m, 1H), 3.77 (dd, 2H, J=11.5, 5.0 Hz), 3.68-3.65 (m, 47H), 3.59-3.57 (m, 2H), 3.50 and 3.25 (ABq, 2×1 H, J=18.5 Hz), 3.47-3.44 (m, 2H), 3.19-3.15 (m, 1H), 2.93 (dd, 1H, J=12.5, 5.0 Hz), 2.74 (d, 1H, J=12.5 Hz), 2.62-2.57 (m, 1H), 2.26-2.22 (m, 2H), 2.16 (s, 3H), 1.71-1.64 (m, 4H), 1.50-1.43 (m, 2H).

### (6S,7S)-benzyl-7-(poly(ethylene glycol)ether-2-(biotinylamino)ethane)-3-methyl-8-oxo-5-thia-1-azabicyclo-[4.2.0]oct-2-ene-2-carboxylic Acid (10) (Cep-1)

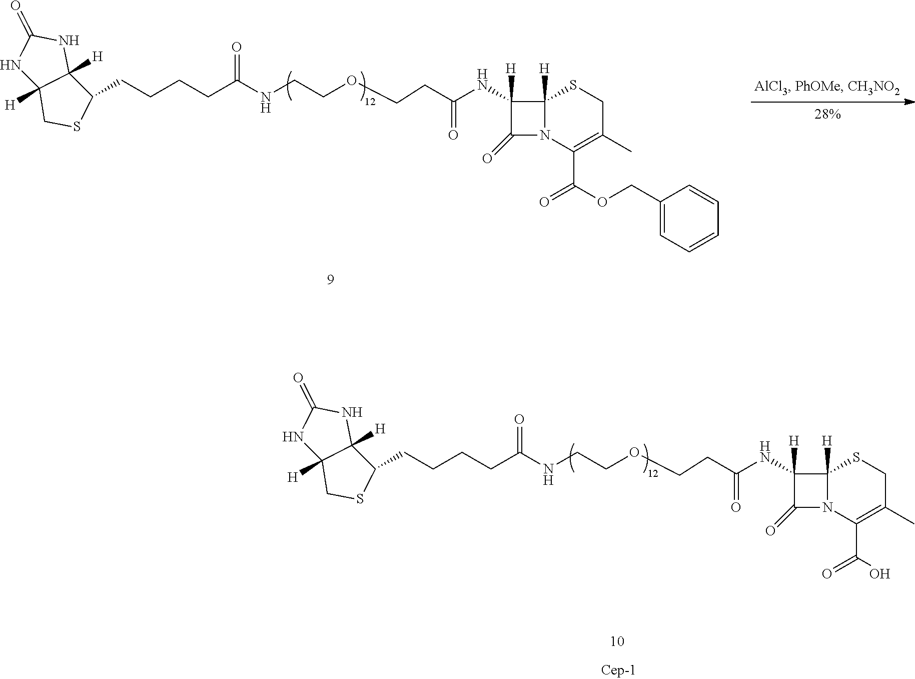

To an ice-bath cooled solution of compound 9 (0.172 gr, 0.152 mmol) and anisole (198 μl, 1.82 mmol) in CH₂Cl₂ (3 ml) was added a cold solution of AlCl₃ (0.122 gr, 0.91 mmol) in MeNO₂. The ice-bath was removed, and the reaction mixture was stirred for 8 h in room temperature. The reaction mixture was diluted with EtOAc (9 ml), and washed with 1 M HCl (3×9 ml) and sat. brine (10 ml). The combined aqueous extracts were washed with Et₂O (10 ml), acidified to pH 1 with 1 M HCl, and re-extracted with EtOAc (3×10 ml). The combined organic layers were washed with satd. brine (10 ml), dried over MgSO₄, and evaporated in vacuo to give compound 10 (Cep-1) which was purified by RP-HPLC (rt=16.5 min, C18 semi-prep column, 20-40% B; A=98% H₂O/Acetonitrile; B=99% Acetonitrile/H₂O). ¹H NMR (D₂O, 500 MHz) δ 5.81 (q, 1H, J=4.5 Hz), 5.49 (dd, 2H, J=20.0, 12.5 Hz), 4.98 (d, 1H, J=5.0 Hz), 4.51-4.448 (m, 1H), 4.29-4.33 (m, 1H), 3.77 (dd, 2H, J=11.5, 5.0 Hz), 3.62-3.58 (m, 47H), 3.59-3.57 (m, 2H), 3.50 and 3.25 (ABq, 2×1 H, J=18.5 Hz), 3.46-3.43 (m, 2H), 3.24-3.19 (m, 1H), 2.89 (dd, 1H, J=12.5, 5.0 Hz), 2.68 (d, 1H, J=12.5 Hz), 2.57-2.5 (m, 1H), 2.2-2.19 (m, 2H), 1.8 (s, 3H), 1.65-1.5 (m, 4H), 1.33-1.28 (m, 2H). ¹³C NMR (D₂O, 125 MHz) δ_{C} 19.8, 25.2, 28.3, 28.5, 29, 34.6, 36.5, 36.7, 39.8, 40.1, 55.6, 56.5, 58.3, 60.5, 62.2, 66.4, 66.7, 69.4, 70.5, 70.8, 123.4, 126.7, 164.7, 165.1, 170.4, 175.6, 177.3

### Synthesis of Enantiomeric Derivatives of Pentapeptide Cell Wall Precursor of S. aureus (Ala-2 and Ala-3)

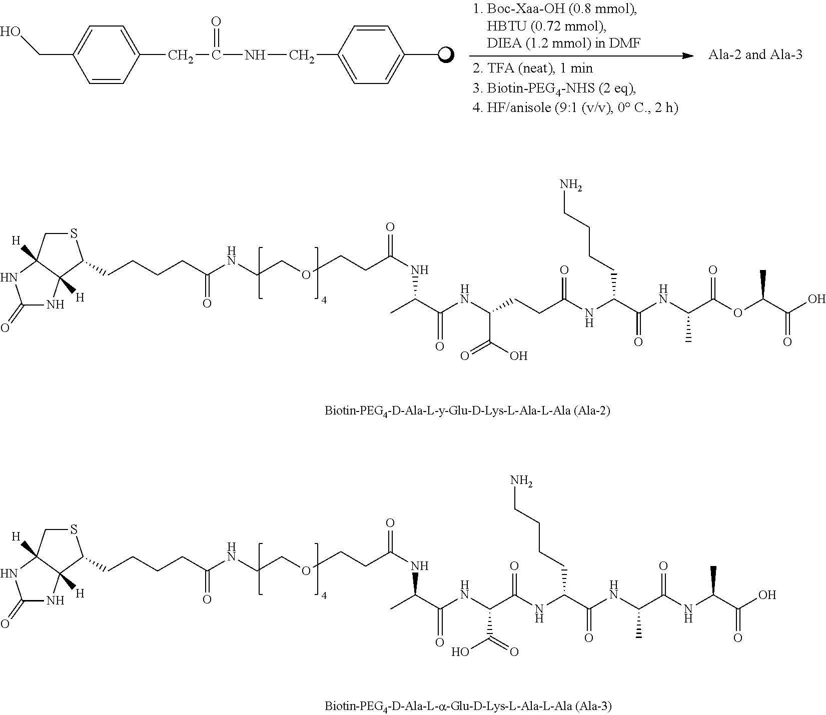

Synthesis of the enantiomeric pentapeptide cell wall precursor (D-Ala-L-γ-Glu-D-Lys-L-Ala-L-Ala) from S. aureus (Ala-2) attached to a biotin moiety via linker is shown above. Enantiomer of pentapeptide precursor of S. aureus with α-Glu instead of γ-Glu harboring an extended backbone, Ala-3, was also synthesized as a control peptide in phage display selection experiments.

D-ala-L-γ-Glu-D-lys-L-Ala-L-Ala, and D-ala-L-α-Glu-D-lys-L-Ala-L-Ala were synthesized manually using the in-situ neutralization protocol for t-Boc (tert-Butoxycarbony) chemistry on 4-Hydroxymethyl-phenylacetamidomethyl (PAM) resin (0.2 mmol scale, 0.85 mmol/g, Chemimpex). Boc-protected amino acids (0.8 mmol) were coupled to resin in DMF with 0-(benzotriazol-1-yl)-N,N,N′,N′-tetramethyluronium hexafluorophosphate (HBTU) (0.72 mmol) and N,N, diisopropylethyl amine (DIEA) (1.2 mmol) for 15 min. N-Boc group were removed by treatment of neat trifluoroacetic acid (TFA) for 1 min twice. After coupling of last amino acid residue, Biotin-PEG4-NHS (2 eq.) linker was coupled to each pentapeptide overnight with DIEA (4 eq.). Peptides were cleaved from PAM-resin by treatment with HF/anisole (9:1 v/v) at 0° C. for 2 h, and then precipitated with cold diethyl ether. Lyophilized crude peptides were purified by reversed-phase HPLC on a C18 column using a linear gradient of increasing concentration of solvent B in solvent A (solvent A: water/acetonitrile/TFA, 98/2/0.1; solvent B: acetonitrile/water/TFA, 99/1/0.1). Purity of peptides were determined by analytical RP-HPLC on a C18 column (5 μm, 4×250 mm) using a linear gradient (5 to 20% solvent B in solvent A) over 30 min at a flow rate of 1 ml min-1. MALDI-TOF mass spectra was recorded using alpha-cyano 4-hydroxycinnaminic acid as matrix. Ala-2, calcd [M+H]+, 962.46, obsd, 962.121; Ala-3, calcd [M+H]+, 962.46, obsd, 962.121.

### Synthesis of Enantiomer of Cell Wall Precursor of Vancomycin-resistant Enterococci (Lac-4)

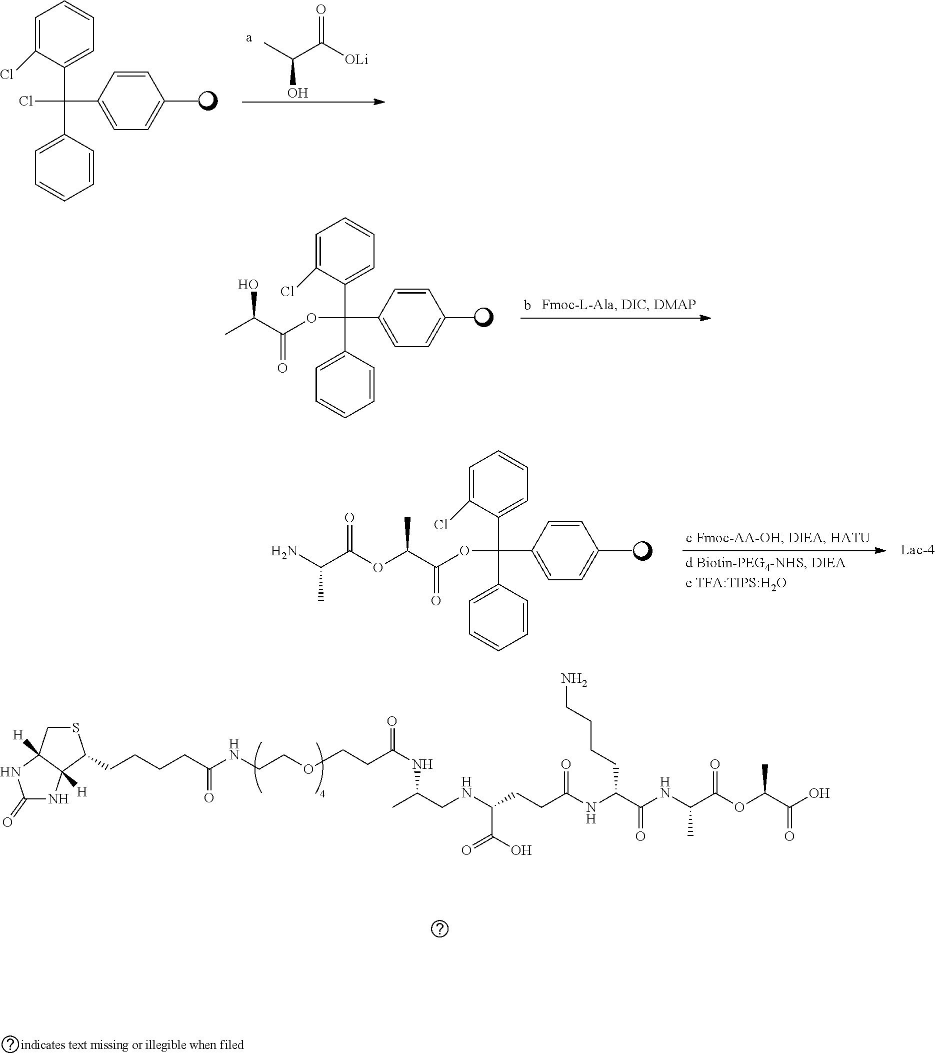

Synthesis of the enantiomer of cell wall analogue of vancomycin-resistant Enterococci (VRE) is shown above. a Synthetic scheme of Lac-4 starting with coupling of L-Lac lithium salt on 2-chlorotrityl resin, and followed by solid-phase peptide synthesis by Fmoc-chemistry. b Structure of Lac-4 used in phage display screening.

D-Ala-L-γ-Glu-D-Lys-L-Ala-L-Lac, Lac-4, was synthesized manually by SPPS using standard Fmoc-chemistry (N-(9-fluorenyl)methoxycarbonyl) on 2-chlorotrityl chloride resin (0.2 mmol scale, 0.8 meq/g, ChemImpex). 1.5 mmol L-Lac lithium salt was coupled directly to 2-chlorotritiyl chloride resin in DMSO:DCM (1:1) overnight. Following day, the next residue Fmoc-L-Ala-OH (1.5 mmol) in DMF was coupled overnight to resin-complex by adding DIC (1.5 mmol) and catalytic amount of DMAP (0.08 mmol). The following amino acids were coupled using standard Fmoc-chemistry, where 0.8 mmol amino acid in DMF was activated by Azabenzotriazol-1-yl-N,N,N′,N′-tetramethyluronium hexafluorophosphate (HATU) (0.72 mmol) and DIEA (1.2 mmol), and coupled to peptide-resin for 30 min. After coupling of last amino acid, Biotin-PEG₄-NHS linker (2 eq.) was coupled to peptide-resin overnight. Biotinylated peptide was cleaved from resin by acidic cleavage cocktail containing TFA/Tri-isopropylsilane (TIPS)/H₂O (95/2.5/2.5) for 3 hours. TFA was evaporated under flow of nitrogen, and the cleaved peptide was precipitated with ice-cold diethyl ether three times. Lyophilized crude peptide was purified by reversed-phase HPLC on a C18 column using a linear gradient of increasing concentration of solvent B in solvent A (solvent A: water/acetonitrile/TFA, 98/2/0.1; solvent B: acetonitrile/water/TFA, 99/1/0.1). Purity was determined by analytical RP-HPLC on a C18 column (5 μm, 4×250 mm) using a linear gradient (10 to 45% solvent B in solvent A) over 30 min at a flow rate of 1 ml min⁻¹. MALDI-TOF mass spectras were recorded using alpha-cyano 4-hydroxycinnaminic acid as matrix. Lac-4, calcd [M+H]⁺, 963.46, obsd, 963.11

Phage Selection with Linear and Cyclic Libraries.

Ph.D.-12, Ph.D.-7 and Ph.D.-C7C Phage Display (Ph.D.) peptide libraries were purchased from New England Biolabs (NEB #E8110S, #E8100S and #E8120S). In the first round of selection, target molecules were incubated with 100-fold diluted (10¹¹ pfu/ml) phage libraries in TBS buffer containing 0.1% Tween-20 for 30 min by agitation in room temperature. The phage/target molecule was then captured on streptavidin-coated magnetic beads (NEB #S1420S) by incubation for 30 min at room temperature, washed six times with TBS containing 0.1% Tween-20. The phage were then eluted by incubation with 0.2 M Glycine-HCl (pH 2.2) for 15 min. After neutralization with 1.0 M Tris-HCl (pH 9.1), phages were propagated by infection of E. coli ER2738 cells that were grown to an optical density of 0.5 at 600 nm (OD₆₀₀) by a 4.5 h incubation at 37° C. at 250 rpm. The infected cells were harvested by centrifugation, plated on Luria-Bertani/tetracycline plates, incubated at 37° C. overnight, and used to produce phage for the next round of selection. Three additional selection rounds were performed with higher concentration of Tween-20, ranging from 0.1% to 0.5% and by use of streptavidin and neutravidin-coated magnetic beads in alternating rounds. The more stringent selection was carried out using 25 ng of target molecule in each round while the high yield selection was performed with reduced amount of target molecule (5 mg, 2 mg, 500 ng, and 100 ng, respectively) in each round. Negative selection was also performed at the second, third and fourth rounds of biopanning to assess enrichment during each round by using solely neutravidin-coated magnetic beads with phage library not containing target molecule in the second and the fourth rounds, and using solely streptavidin-coated magnetic beads with phage library not containing the target molecule in the third round.

### Blue/White Plaque Assays for Phage Titering.

After each round of biopanning, the number of phage in each unamplified and amplified eluate was determined by blue/white plaque assay for plaque forming units as in NEB protocol. A single colony from re-streaked host cell (E. coli ER2738) was inoculated in LB (10 μg/ml tetracycline) medium at 37° C. by shaking at 250 rpm till cells growth to mid-log phase, OD₆₀₀ of 0.5. Meanwhile, serial ten-fold dilutions of phage (10² to 10⁴ serial dilutions for unamplified eluates, and 10⁸ to 10¹¹ serial solution for amplified eluates) in LB medium were prepared. Then, 10 μl of each phage dilutions were infected with 200 μl of host cell E. coli culture (OD₆₀₀ of 0.5), and spread onto LB medium/X-gal/IPTG plates (Teknova, San Diego) at 37° C. overnight. Subsequently, blue plaques appeared as a result of cleavage of XGal by β-galactosidase encoded by the lacZ gene in the M13 phage construct in the presence of inducer IPTG on LB medium/X-gal/IPTG plates, and were counted and phage titers were calculated as plaque forming unit (pfu/ml).

### Plaque Purification for DNA Sequencing and Phage-ELISA.

A single colony of E. coli ER2537 was inoculated overnight in 10 ml of LB (10 mg/ml tetracycline) medium at 37° C./250 rpm, and then diluted 100-fold in LB medium for plaques to be tested. Randomly picked single well-separated blue plaques from LB/IPTG/Xgal plates were transferred into 1 ml of diluted E. coli culture, and amplified by incubation for 4.5 h at 37° C./250 rpm. Following this, the amplified phage plaque culture was centrifuged at 14,000 rpm for 2×1 min at room temperature. The upper 600 μl of the supernatant was then mixed with 600 μl of 50% glycerol in a new microcentrifuge tube. Individual phage stocks were kept at −20° C. till further use for phage-ELISA experiments and DNA sequencing.

### Phage ELISA.

Plates (96-well, Nunc Maxisorp #44-2404-21, Fisher Scientific) were coated with 100 μg/ml streptavidin solution overnight at 4° C. Following this, the coated wells were washed 4× with TBS containing 0.5% Tween-20, blocked with 0.1 M NaHCO₃ (pH 8.6), 5 mg/ml BSA for 2 h in a refrigerator (4° C.), and washed 6× with TBS containing 0.5% Tween-20. Biotinylated target molecules (200 pmol) were added into each well and allowed to bind for 2 h at room temperature followed by incubation with amplified phage for 2 h at room temperature. After washing 10×, a 1:5000 diluted Horseradish Peroxidase-conjugated anti-M13 monoclonal antibody solution (GE Healthcare, #27-9421-01) was added to each well, followed by incubation for 1 h with agitation at room temperature. The absorbance of each well was recorded at 405 nm using a microplate reader after incubation with ABTS substrate solution (azino-bis(3-ethylben-zothiazole) sulfonic acid), pH 4, (Sigma Aldrich, #A-1888) with 30% H₂O₂. Streptavidin and BSA coated wells as well as empty wells were used as negative controls for binding specificity by carrying out the same protocols without use of target molecules.

### Bicyclic Phage Library Production and Amplification.

Each phage library was amplified till to reach OD₆₀₀ of 0.1 overnight by vigorous shaking at 250 rpm/30° C. in 2×YT/chloramphenicol medium. The amplified phage library was purified by precipitation with 1/4 volume of ice-cold 20% (w/v) PEG8000/2.5 M NaCl in an ice-bath for 1 h, and purified phage were centrifuged at 8500 rpm for 45 min at 4° C. to obtain phage pellets. The phage pellets were then suspended in the reaction buffer (20 mM NH₄HCO₃, 5 mM EDTA, pH 8.0) for 30 min, centrifuged at 4000 rpm for 15 min at 4° C., and the supernatant containing phage particles was used to assess the number of phage recovered. To PEG-purified phage solution, tris(2-carboxyethyl)phosphine (TCEP) was added to get 1 mM final concentration and incubated for 1 h in a hot water bath at 42° C. to reduce cysteine disulfide bridges in phage proteins. This mixture was cooled for 5 min in an ice-bath, then centrifuged at 4000 rpm at 4° C. until the volume was reduced to 1 ml with Vivaspin-20 100,000 PES filter tubes (Sartorius Stedim, #VS2042). All reduced phage particles were recovered by washing with reaction buffer twice and excess TCEP solution was washed away. The volume of TCEP-reduced phage was adjusted to 8 ml with ice-cold degassed reaction buffer, and incubated with 100 μM 1,3,5-tris(bromomethyl)benzene (TBMB) in acetonitrile for 1 h in a hot water bath at 30° C. for bicyclization of phage library. Then, bicyclic phage libraries were purified by precipitation with 20% (w/v) PEG8000/2.5 M NaCl solution on ice-bath for 30 min. Phage pellets were suspended in 1 ml blocking buffer (TBS buffer with 1% (w/v) BSA and 0.1% (v/v) Tween-20) to use for phage display selection at 4° C.

Phase Selection with Bicyclic Phase Display Libraries.

Biotinylated target molecules (5 mg) dissolved in binding buffer (10 mM TBS, 150 mM NaCl, pH 7.4) were incubated with streptavidin-coated magnetic beads (SA-beads) for 30 min. Post incubation, excess biotinylated target molecules were washed away, and target-coupled SA-beads were added to a blocking buffer (TBS buffer with 1% (w/v) BSA and 0.1% (v/v) Tween-20) at room temperature for 2 h. After washing target-coupled SA-beads three times, overnight pre-blocked bicyclic phage library were added and allowed to stand for 1 h at room temperature by slow agitation. The unbound phage were washed away, eluted with 0.2 M Glycine-HCl (pH 2.2) for 10 min and neutralized with 1.0 M Tris-HCl (pH 8.0). To an overnight-grown E. coli TG1 culture was added in 2×YT medium at 37° C./250 rpm till OD₆₀₀ reached 0.4, and the eluted phage were then added to the culture for amplification at 37° C. After this step, the cells were pelleted by centrifugation at 4000 rpm for 5 min at 4° C., and re-suspended in 2×YT medium, then plated on 2×YT/30 μg/ml chloramphenicol agar plates at 37° C. overnight. The following day, cells on the agar plates were harvested with 2×YT medium/20% glycerol stored at −80° C. to use in the next round of selection. The second round of selection was performed by using neutravidin-coated magnetic beads instead of streptavidin in order to prevent the enrichment of streptavidin-specific peptide binders. The amount of target molecules used were limited to 2 mg (from 5 mg) to increase stringency. Control selections were performed at each round without using target molecules to calculate enrichment factors.

### Synthesis of Linear D-Peptides.

Peptides were chemically synthesized by SPPS using Fmoc-chemistry on Rink-4-(2′,4′-dimethoxyphenyl-Fmoc-aminomethyl)-phenoxy resin (Rink Amide) (0.1 mmol scale, 0.33 meq/g, ChemImpex). Amino acids were coupled by addition of 0.8 mmol Fmoc-protected amino acid, 0.72 mmol HATU, and 1.2 mmol DIEA in 2 ml DMF for 45 min. Peptides were cleaved from the resin under reducing conditions (90% TFA, 2.5% ethanedithiol, 2.5% phenol, 2.5% thioanisole, 2.5% H₂O). TFA was evaporated under flow of nitrogen, and the cleaved peptides were precipitated in dry-ice cold diethylether three times, and centrifuged. Crude peptides were dried under high vacuum, dissolve in 50% acetonitrile/water and lyophilized. Lyophilized crude peptides were purified by reversed-phase HPLC on a C18 column using linear gradient of increasing concentration of solvent B in solvent A (from 10% to 45%). The purity of peptides was confirmed by analytical RP-HPLC, and MALDI-TOF mass spectras were recorded using alpha-cyano 4-hydroxycinnaminic acid as matrix.

### Synthesis of Cyclic D-Peptides.

After synthesis and purification of linear peptides using the above described protocol, cyclization of peptides was achieved by air oxidation to yield disulfide bonds. Purified peptides (5 mg) were dissolved in 5 ml of oxidation buffer (20 mM NH₄CO₃, pH 8) and incubated overnight while exposed to air. The resultant oxidized peptides were lyophilized and purity was confirmed by analytical RP-HPLC and MWs were confirmed using MALDI-TOF mass spectroscopy.

### Synthesis of Bicyclic D-Peptides.

Linear D-peptides having three cysteines were synthesized by SPPS using standard Fmoc-chemistry on Rink Amide resin (0.1 mmol scale, 0.33 meq/g). After cleavage of linear peptides in reducing cleavage cocktail, crude peptides were lyophilized. 1 mM solution of the crude peptides was dissolved in 0.1 M NH₄HCO₃, pH 8, and reacted by addition of 1.2 mM 1,3,5-tris-(bromomethyl)benzene (TBMB) in acetonitrile for 1 h in hot water bath at 30° C. This solution was lyophilized and the crude peptides were purified by RP-HPLC using a linear gradient from 10 to 60% solvent B over 40 min and purity was confirmed by analytical RP-HPLC and MWs were determined with MALDI-TOF mass spectroscopy that all showed +114 Da peaks indicative of bicyclic products.

### Minimum Inhibitory Concentration (MIC).

MICs were determined according to a 96-well microdilution protocol outlined by the Clinical and Laboratory Standard Institute (CLSI) guideline with modifications for peptide antibiotics reported by Wiegand et al.³⁵ Two-fold serial peptide dilutions were performed to go from 2560 μg/ml to 5 μg/ml in0.2% BSA and 0.01% acetic acid buffer in 96-well polypropylene plates (COSTAR, #3879). Dilution of bacterial suspensions (1:200) of different bacterial strains that were grown until 0.5 McFarland standard were prepared to obtain a final inoculum concentration of 8×10⁵ CFU/ml in Mueller-Hinton broth. Bacterial inoculum (900 were cultured with 10 μl of two-fold serial dilution solutions of peptides at 37° C. for 18 h. MICs were determined visually on the basis of turbidity as the lowest concentration inhibiting bacterial growth following CLSI guideline. MH broth without antibiotic and bacterial suspension was used as a growth control, as well as vancomycin and melittin were used as control peptide antibiotics in each assay. Each assay was repeated for E. coli ATCC 25992, B. subtilis ATCC 6633, S. aureus ATCC 6538, MRSA ATCC 43300, E. faecalis ATCC 29121, VRE ATCC 51299, VRE ATCC 51559 in triplicate at three independent days.

### Time-Kill Kinetic Assays.

The bacteria-killing kinetics of peptides with S. aureus was measured at 5×MICs, and vancomycin was used as a positive control. An overnight culture of bacterial cells was re-suspended in fresh MH broth at density of 10⁷ cells/ml, and cultured with peptides at 37° C. Viable colony forming units (CFUs) were counted by performing tenfold serial dilutions of the aliquot sample at intervals of 0, 1, 2, 4, 8, 12, and 24 hours after inoculation, and plated on MHB agar plates. Time-kill curves were plotted as Log₁₀ (cfu ml⁻¹) versus time functions.

### Mammalian Cytotoxicity (MTT) Assay.

Cervical cancer cells (HeLa) were cultured in Dulbecco's Modified Eagle's medium (DMEM) (Invitrogen #11995-073) supplemented with 10% bovine calf serum at 37° C., 5% CO₂, and seeded (5×10³ cells/well) in 96-well plate flat-bottomed plate for 24 h before the assay. Peptides ranging in concentration from 0n/ml to 128 μg/ml in DMEM were incubated with HeLa cells at 37° C. for 24 h. After washing the cells with PBS, the cells were subjected to the standard yellow tetrazolium (3-(4,5-dimethylthiazol-2-yl)-2,5-diphenyltetrazolium bromide (MTT) assay protocol at final concentration of 0.5 mg/ml for 4 h in a humidified 5% CO₂ atmosphere at 37° C. MTT solution was then aspirated, and the purple formazan crystals were resuspended in DMSO by incubation for 5 min at 37° C. The absorbance at 540 nm was then read using a microplate reader. Viability control (100% viability) was assigned to samples with only HeLa cells and DMEM media, 0% viability was assigned to samples with 1% added Triton-X-100 and no peptide. Experiments were performed as triplicates at different days.

### Hemolytic Activity.

Fresh red human blood cells (hRBC) were washed and centrifuged at 3000 rpm for 5 min at 4° C. with PBS until a clear solution was obtained. Two-fold serial dilutions of each peptide in PBS from 0.5 μg/ml to 256 μg/ml were incubated with 0.25% (v/v) hRBC suspension at 37° C. for 1 h, and then centrifuged for 5 min at 3500 rpm in 96-well plate. 50 μl of the supernatant from each well was diluted with 50 μl pure water, and the OD at 415 nm was measured using microplate reader. Melittin at the same concentrations (200 μg/ml for complete lysis) was used as a positive control. Percent hemolysis was calculated according to the equation:

$$Percent hemolysis = \frac{( {OD}_{415} peptide - {OD}_{415} buffer )}{( {OD}_{415} complete hemolysis - {OD}_{415} buffer )} \times 100$$

where complete hemolysis was defined as the average hemolysis of all wells containing a final concentration of melittin ranging from 50 to 400 μg/ml. Experiments were performed as triplicates at different days.

### Protease Stability Assay.

Pancreatin (Sigma, 100 μg/ml solution in PBS) was added to peptide solutions to give a final concentration of 1 mg/ml peptide and 50 μg/ml of pancreatin, and maintained at 37° C. Aliquots of samples (15 μl) were quenched with 4% (v/v) TFA for 5 min at selected time points (0, 15, 30, 60, 120, 180, 240, 360 min and overnight). Samples were analyzed using RP-HPLC immediately to assess the proteolytic activity.

### Serum Stability Assay.

Human serum was prepared by centrifuging the coagulated human blood at 3000 rpm for 15 min to get a clear supernatant. Peptide solutions (1 mg/ml) were diluted ten-fold in human serum, and incubated at 37° C. Aliquots from serum/peptide solution were taken at different time points, 0, 5, 30, 60, 120, 240 min and overnight, and quenched with 4% TFA at 4° C. for 15 min, and centrifuged for 15 min at 13,000 rpm. The supernatant was analyzed using RP-HPLC immediately.

Accumulation of UDP-N-acetylmuramyl-pentapeptide precursor from S. aureus Accumulation of peptidoglycan precursor of S. aureus (ATCC 6633) was analyzed in the presence of peptide antibiotics. Bacteria were grown at 37° C. till OD₆₀₀ of 0.5 was attained. Ten times MICs of peptide antibiotics were added per ml of grown bacterial culture and allowed to stand for 1 h. After cooling the cells on ice, they were harvested by centrifugation at 4° C. for 15 min. Bacterial cells were suspended in distilled water, and then slowly stirred into boiling water for 15 min. The suspension was allowed to cool first at room temperature, then on ice. After centrifugation at 4° C. for 1 h, the supernatant was lyophilized. The lyophilized sample was dissolved in water, and pH was adjusted to 2 by addition of 20% H₃PO₄. The white precipitate was removed by centrifugation, and the supernatant was immediately subjected to RP-HPLC analysis and MWs were assessed by MALDI-TOF mass spectroscopy.

TABLE 7
Phage titers using Cep-1 during phage display screening
with bicyclic peptide libraries including negative selections.

| | | Round 1 | Round 2 | Enrichment |
|---|---|---|---|---|
| Library A | N.S. | 2.8 × 10⁴ | 1.6 × 10⁴ | — |
| | Cep-1 | 1.9 × 10⁴ | 3.6 × 10⁷ | 1.9 × 10³ |
| Library B | N.S. | 1.30 × 10⁴ | 3.50 × 10³ | — |
| | Cep-1 | 2.75 × 10⁵ | 1.25 × 10⁶ | 36 × 10² |

TABLE 8
Phage titers using Ala-2 during phage display screening
with bicyclic peptide libraries including negative selections.

| | | Round 1 | Round 2 | Enrichment |
|---|---|---|---|---|
| Library A | N.S. | 1.8 × 10³ | 1.1 × 10² | — |
| | Ala-2 | 4.7 × 10⁴ | 3.0 × 10⁴ | 7.5 × 10³ |
| Library B | N.S. | 2.6 × 10⁴ | 1.10 × 10⁵ | — |
| | Ala-2 | 2.0 × 10⁵ | 1.75 × 10⁵ | 1.6 × 10³ |

TABLE 9
Phage titers of Lac-4 during phage display screening
with bicyclic peptide libraries including negative selections.

| | | Round 1 | Round 2 | Enrichment |
|---|---|---|---|---|
| Library A | N.S. | 1.3 × 10⁴ | 1.1 × 10⁴ | — |
| | Lac | 6 × 10⁴ | 4.6 × 10⁶ | 4.1 × 10² |
| Library B | N.S. | 3.1 × 10³ | 1.6 × 10⁴ | — |
| | Lac | 1.7 × 10⁴ | 3.9 × 10⁶ | 2.4 × 10² |

### REFERENCES

1. Levy, S. B.; Marshall, B., Antibacterial resistance worldwide: causes, challenges and responses. Nat. Med. 2004, 10, S122-S129.
2. Plata, K.; Rosato, A. E.; Wegrzyn, G., Staphylococcus aureus as an infectious agent: overview of biochemistry and molecular genetics of its pathogenicity. Acta Biochim. Pol. 2009, 56, 597-612.
3. Li, J. W. H.; Vederas, J. C., Drug Discovery and Natural Products: End of an Era or an Endless Frontier? Science 2009, 325, 161-165.
4. Livermore, D. M. et al., Discovery research: the scientific challenge of finding new antibiotics. J. Antimicrob. Chemother. 2011, 66, 1941-1944.
5. Theuretzbacher, U., Resistance drives antibacterial drug development. Curr. Opin. Pharmacol. 2011, 11, 433-438.
6. Walsh, C. T.; Wencewicz, T. A., Antibiotics: Challenges, Mechanisms, Opportunities. 1st Edition ed.; ASM Press: 2016.
7. Borek, F.; Stupp, Y.; Fuchs, S.; Sela, M., Relation between optical configuration and immunogenicity of synthetic polypeptides. Biochem. J. 1965, 96, 577-&.
8. Dintzis, H. M.; Symer, D. E.; Dintzis, R. Z.; Zawadzke, L. E.; Berg, J. M., A comparison of the immunogenicity of a pair of enantiomeric proteins. Proteins 1993, 16, 306-308.
9. Gill, T. J.; Doty, P.; Gould, H. J., Synthetic antigens composed exclusively of L- or D-amino acids. I. Effect of optical configuration on immunogenicity of synthetic polypeptides in mice. Nature 1963, 197, 746-&.
10. Janeway, C. A.; Sela, M., Synthetic antigens composed exclusively of L- or D-amino acids. I. Effect of optical configuration on immunogenicity of synthetic polypeptides in mice. Immunology 1967, 13, 29-&.
11. Pappenheimer, J. R.; Dahl, C. E.; Karnovsky, M. L.; Maggio, J. E., Intestinal-absorption and excretion of octapeptides composed of D-amino-acids. Proc. Natl. Acad. Sci. U.S.A 1994, 91, 1942-1945.
12. Pappenheimer, J. R.; Karnovsky, M. L.; Maggio, J. E., Absorption and excretion of undegradable peptides: Role of lipid solubility and net charge. J. Pharmacol. Exp. Ther. 1997, 280, 292-300.
13. Sidhu, S. S., Phage display in pharmaceutical biotechnology. Curr. Opin. Biotechnol. 2000, 11, 610-616.
14. Wilson, D. R.; Finlay, B. B., Phage display: applications, innovations, and issues in phage and host biology. Can. J. Microbiol. 1998, 44, 313-329.
15. Barbu, E. M.; Ganesh, V. K.; Gurusiddappa, S.; Mackenzie, R. C.; Foster, T. J.; Sudhof, T. C.; Hook, M., beta-Neurexin Is a Ligand for the Staphylococcus aureus MSCRAMM SdrC. PLoS Pathog. 2010, 6.
16. Huang, W. Z.; Zhang, Z.; Palzkill, T., Design of potent beta-lactamase inhibitors by phage display of beta-lactamase inhibitory protein. J. Biol. Chem. 2000, 275, 14964-14968.
17. Soykut, E. A.; Dudak, F. C.; Boyaci, I. H., Selection of staphylococcal enterotoxin B (SEB)binding peptide using phage display technology. Biochem. Biophys. Res. Commun. 2008, 370, 104-108.
18. Yang, G.; Cheng, H. C.; Liu, C.; Xue, Y. N.; Gao, Y. P.; Liu, N. L.; Gao, B.; Wang, D. P.; Li, S. R.; Shen, B. F.; Shao, N. S., Inhibition of Staphylococcus aureus pathogenesis in vitro and in vivo by RAP-binding peptides. Peptides 2003, 24, 1823-1828.
19. Schumacher, T. N. M.; Mayr, L. M.; Minor, D. L.; Milhollen, M. A.; Burgess, M. W.; Kim, P. S., Identification of D-peptide ligands through mirror-image phage display. Science 1996, 271, 1854-1857.
20. Eckert, D. M.; Malashkevich, V. N.; Hong, L. H.; Can, P. A.; Kim, P. S., Inhibiting HIV-1 entry: Discovery of D-peptide inhibitors that target the gp41 coiled-coil pocket. Cell 1999, 99, 103-115.
21. Klussmann, S.; Nolte, A.; Bald, R.; Erdmann, V. A.; Furste, J. P., Minor-image RNA that binds D-adenosine. Nat. Biotechnol. 1996, 14, 1112-1115.
22. Kozlov, I. A.; Mao, S. L.; Xu, Y.; Huang, X. F.; Lee, L.; Sears, P. S.; Gao, C. S.; Coyle, A. R.; Janda, K. D.; Wong, C. H., Synthesis of solid-supported mirror-image sugars: A novel method for selecting receptors for cellular-surface carbohydrates. ChemBioChem 2001, 2, 741-746.
23. Liu, H. M.; Funke, S. A.; Willbold, D., Transport of Alzheimer Disease Amyloid-beta-Binding D-Amino Acid Peptides across an In Vitro Blood-Brain Barrier Model. Rejuv. Res. 2010, 13, 210-213.
24. Wiesehan, K.; Buder, K.; Linke, R. P.; Patt, S.; Stoldt, M.; Unger, E.; Schmitt, B.; Bucci, E.; Willbold, D., Selection of D-Amino-Acid peptides that bind to Alzheimer's disease amyloid peptide A beta(1-42) by mirror image phage display. ChemBioChem 2003, 4, 748-753.
25. Hart, P.; Wood, T. M.; Tehrani, K.; van Harten, R. M.; Sleszynska, M.; Rebollo, I. R.; Hendrickx, A. P. A.; Willems, R. J. L.; Breukink, E.; Martin, N. I., De novo identification of lipid II binding lipopeptides with antibacterial activity against vancomycin-resistant bacteria. Chem. Sci. 2017, 8, 7991-7997.
26. Williamson, M. P.; Williams, D. H.; Hammond, S. J., Interactions of Vancomycin and Ristocetin with peptides as a model for protein-binding. Tetrahedron 1984, 40, 569-577.
27. Tipper, D. J.; Strominger, J. L., Mechanism of action of penicillins—A proposal based on their structural similarity to acyl-D-alanyl-D-alanine. Proc. Natl. Acad. Sci. U.S.A 1965, 54, 1133-+.
28. Fekner, T.; Baldwin, J. E.; Adlington, R. M.; Jones, T. W.; Prout, C. K.; Schofield, C. J., Syntheses of (6S)-cephalosporins from 6-aminopenicillanic acid. Tetrahedron 2000, 56, 6053-6074.
29. Fekner, T.; Baldwin, J. E.; Adlington, R. M.; Schofield, C. J., Syntheses of (6S,7S)- and (6S,7R)-deacetoxycephalosporanic acids from 6-aminopenicillanic acid. Chem. Commun. 1996, 1989-1990.
30. Popieniek, P. H.; Pratt, R. F., A fluorescent ligand for binding-studies with glycopeptide antibiotics of the vancomycin class. Anal. Biochem. 1987, 165, 108-113.
31. Popieniek, P. H.; Pratt, R. F., Kinetics and mechanism of binding of specific peptides to vancomycin and other glycopeptide antibiotics. J. Am. Chem. Soc. 1991, 113, 2264-2270.
32. Heinis, C.; Rutherford, T.; Freund, S.; Winter, G., Phage-encoded combinatorial chemical libraries based on bicyclic peptides. Nat. Chem. Biol. 2009, 5, 502-507.
33. Angelini, A.; Cendron, L.; Chen, S. Y.; Touati, J.; Winter, G.; Zanotti, G.; Heinis, C., Bicyclic Peptide Inhibitor Reveals Large Contact Interface with a Protease Target. ACS Chem. Biol. 2012, 7, 817-821.
34. Baeriswyl, V.; Calzavarini, S.; Gerschheimer, C.; Diderich, P.; Angelillo-Scherrer, A.; Heinis, C., Development of a Selective Peptide Macrocycle Inhibitor of Coagulation Factor XII toward the Generation of a Safe Antithrombotic Therapy. J. Med. Chem. 2013, 56, 3742-3746.
35. Baeriswyl, V.; Rapley, H.; Pollaro, L.; Stace, C.; Teufel, D.; Walker, E.; Chen, S. Y.; Winter, G.; Tite, J.; Heinis, C., Bicyclic Peptides with Optimized Ring Size Inhibit Human Plasma Kallikrein and its Orthologues While Sparing Paralogous Proteases. ChemMedChem 2012, 7, 1173-1176.
36. Diderich, P.; Heinis, C., Phage selection of bicyclic peptides binding Her2. Tetrahedron 2014, 70, 7733-7739.
37. Urech-Varenne, C.; Radtke, F.; Heinis, C., Phage Selection of Bicyclic Peptide Ligands of the Notch1 Receptor. ChemMedChem 2015, 10, 1754-1761.
38. Wiegand, I.; Hilpert, K.; Hancock, R. E. W., Agar and broth dilution methods to determine the minimal inhibitory concentration (MIC) of antimicrobial substances. Nat. Protoc. 2008, 3, 163-175.
39. Griffith, R. S., Vancomycin Use—An Historical Review. J. Antimicrob. Chemother. 1984, 14, 1-5.
40. Dai, D.; Ishiguro, E. E., MurH, a new genetic-locus in Escherichia Coli involved in cell-wall peptidoglycan biosynthesis. J. Bacteriol. 1988, 170, 2197-2201.
41. Walsh, C. T.; Fisher, S. L.; Park, I. S.; Prahalad, M.; Wu, Z., Bacterial resistance to vancomycin: Five genes and one missing hydrogen bond tell the story. Chem. Biol. 1996, 3, 21-28.
42. Allen, N. E.; Hobbs, J. N.; Nicas, T. I., Inhibition of peptidoglycan biosynthesis in vancomycin-susceptible and -resistant bacteria by a semisynthetic glycopeptide antibiotic. Antimicrob. Agents Chemother. 1996, 40, 2356-2362.
43. Bischoff, D.; Pelzer, S.; Bister, B.; Nicholson, G. J.; Stockert, S.; Schirle, M.; Wohlleben, W.; Jung, G.; Sussmuth, R. D., The biosynthesis of vancomycin-type glycopeptide antibiotics—The order of the cyclization steps. Angew. Chem.-Int. Edit. 2001, 40, 4688-+.
44. Chamorro, C.; Liskamp, R. M. J., Selection of synthetic receptors capable of sensing the difference in binding of D-Ala-D-Ala or D-Ala-D-Lac ligands by screening of a combinatorial CTV-based library. Tetrahedron 2004, 60, 11145-11157.
45. Chiosis, G.; Boneca, I. G., Selective cleavage of D-Ala-D-Lac by small molecules: Re-sensitizing resistant bacteria to vancomycin. Science 2001, 293, 1484-1487.
46. Crane, C. M.; Boger, D. L., Synthesis and Evaluation of Vancomycin Aglycon Analogues That Bear Modifications in the N-Terminal D-Leucyl Amino Acid. J. Med. Chem. 2009, 52, 1471-1476.
47. Griffin, J. H.; Linsell, M. S.; Nodwell, M. B.; Chen, Q. Q.; Pace, J. L.; Quast, K. L.;

Krause, K. M.; Farrington, L.; Wu, T. X.; Higgins, D. L.; Jenkins, T. E.; Christensen, B. G.; Judice, J. K., Multivalent drug design. Synthesis and in vitro analysis of an array of vancomycin dimers. J. Am. Chem. Soc. 2003, 125, 6517-6531.
48. Isomura, S.; Ashley, J. A.; Wirsching, P.; Janda, K. D., Antibody-catalyzed cleavage of the D-Ala-D-lac depsipeptide: An immunological approach to the problem of vancomycin resistance. Bioorg. Med. Chem. Lett. 2002, 12, 861-864.
49. Jones, R. N.; Barret, M. S.; Erwin, M. E., In vitro activity and spectrum of LY333328, a novel glycopeptide derivative. Antimicrob. Agents Chemother. 1997, 41, 488-493.
50. Ma, N. C.; Jia, Y. X.; Liu, Z. S.; Gonzalez-Zamora, E.; Bois-Choussy, M.; Malabarba, A.; Brunati, C.; Zhu, J., Design and synthesis of macrocycles active against vancomycin-resistant enterococci (VRE): the interplay between D-Ala-D-Lac binding and hydrophobic effect. Bioorg. Med. Chem. Lett. 2005, 15, 743-746.
51. Maffioli, S. I.; Ciabatti, R.; Romano, G.; Marzorati, E.; Preobrazhenskaya, M.; Pavlov, A., Synthesis and antibacterial activity of alkyl derivatives of the glycopeptide antibiotic A40926 and their amides. Bioorg. Med. Chem. Lett. 2005, 15, 3801-3805.
52. Nakama, Y.; Yoshida, O.; Yoda, M.; Araki, K.; Sawada, Y.; Nakamura, J.; Xu, S.; Miura, K.; Maki, H.; Arimoto, H., Discovery of a Novel Series of Semisynthetic Vancomycin Derivatives Effective against Vancomycin-Resistant Bacteria. J. Med. Chem. 2010, 53, 2528-2533.
53. Pavlov, A. Y.; Berdnikova, T. F.; Olsufyeva, E. N.; Lazhko, E. I.; Malkova, I. V.; Preobrazhenskaya, M. N.; Testa, R. T.; Petersen, P. J., Synthesis and biological-activity of derivatives of glycopeptide antibiotics eremomycin and vancomycin nitrosated, acylated or carbamoylated at the N-terminal. J. Antibiot. 1993, 46, 1731-1739.
54. Rao, J. H.; Whitesides, G. M., Tight binding of a dimeric derivative of vancomycin with dimeric L-Lys-D-Ala-D-Ala. J. Am. Chem. Soc. 1997, 119, 10286-10290.
55. Xie, J.; Pierce, J. G.; James, R. C.; Okano, A.; Boger, D. L., A Redesigned Vancomycin Engineered for Dual D-Ala-D-Ala and D-Ala-D-Lac Binding Exhibits Potent Antimicrobial Activity Against Vancomycin-Resistant Bacteria. J. Am. Chem. Soc. 2011, 133, 13946-13949.
56. Xing, B. G.; Yu, C. W.; Ho, P. L.; Chow, K. H.; Cheung, T.; Gu, H. W.; Cai, Z. W.; Xu, B., Multivalent antibiotics via metal complexes: Potent divalent vancomycins against vancomycin-resistant enterococci. J. Med. Chem. 2003, 46, 4904-4909.
57. Xu, R.; Greiveldinger, G.; Marenus, L. E.; Cooper, A.; Ellman, J. A., Combinatorial library approach for the identification of synthetic receptors targeting vancomycin-resistant bacteria. J. Am. Chem. Soc. 1999, 121, 4898-4899.
58. Yao, N. H.; Liu, G.; He, W. Y.; Niu, C. Q.; Carlson, J. R.; Lam, K. S., Solid-phase synthesis and antibacterial evaluations of N-demethylvancomycin derivatives. Bioorg. Med. Chem. Lett. 2005, 15, 2325-2329.
59. Yarlagadda, V.; Akkapeddi, P.; Manjunath, G. B.; Haldar, J., Membrane Active Vancomycin Analogues: A Strategy to Combat Bacterial Resistance. J. Med. Chem. 2014, 57, 4558-4568.
60. Zhanel, G. G.; Calic, D.; Schweizer, F., New lipoglycopeptides: a comparative review of dalbavancin, oritavancin and telavancin (vol 70, pg 859, 2010). Drugs 2011, 71, 526-526.
61. Zasloff, M., Antimicrobial peptides of multicellular organisms. Nature 2002, 415, 389-395.
62. Meister, A., Biochemistry of the amino acids. 2d ed. ed.; New York, Academic Press: New York, 1965.
63. Boman, H. G., Antibacterial peptides: basic facts and emerging concepts. J. Intern. Med. 2003, 254, 197-215.
64. Meng, H.; Kumar, K., Antimicrobial activity and protease stability of peptides containing fluorinated amino acids. J. Am. Chem. Soc. 2007, 129, 15615-15622.

### Enumerated Embodiments

It shall be understood that the preceding examples are intended to demonstrate how the compositions and methods of the disclosure may be practiced, and are not intended to be limiting as to the scope of the disclosed subject matter.

The subject matter of the present disclosure is also exemplified by the following enumerated embodiments:

Embodiment 1. An isolated peptide comprising an amino acid sequence comprising, consisting of, or having at least 85% identity to the amino acid sequence of a peptide of any of Tables 1-6.
Embodiment 2. The peptide of embodiment 1, wherein each amino acid in the peptide is a D-amino acid.
Embodiment 3. The peptide of embodiment 1 or 2, wherein the peptide comprises one or more sequence motifs selected from TTL, I/L-S/T, Q/N-S/T, LQ, G-S/T/V, G-X-S/T, G-X-S, L-K/R, R-L/V, RV, VLS, N/Q-K/R, GQS, G-S/V, GQV, QL, SSV, T/P-Q, YQ-S/L, and QRL, wherein each X represents any D- or L-amino acid.
Embodiment 4. The peptide of embodiment 3, wherein each X represents any D-amino acid.
Embodiment 5. The peptide of any one of embodiments 1-4, wherein the peptide comprises an amino acid sequence selected from VGSNLRLLHQWK, VHWDFRQWWQPS, KCCYTLP, VLTRCCG, TGFLVNV, HYIDFRW, CLKLGEKWC, CRGATPMSC, CSISSLTHC, CLWSTGATC, CDNIMTPVC, CQTDVCQRTIC, CSLITQCGGVGC, CQYNDCDMLHC, CLSQFCVIDC, CGGGICRTHNC, CTRVCSSSQLYC, and CGAQGCFGVQSC.
Embodiment 6. The peptide of any one of embodiments 1-5, wherein the peptide is linear, cyclic, or bicyclic.
Embodiment 7. An isolated polynucleotide encoding a peptide of embodiment 1.
Embodiment 8. A vector comprising the isolated polynucleotide of embodiment 7.
Embodiment 9. A host cell comprising the vector of embodiment 8.
Embodiment 10. A method of treating a bacterial infection in a subject, said method comprising administering to said subject a therapeutically-effective amount of a peptide of any one of embodiments 1-6.
Embodiment 11. The method of embodiment 10, wherein said subject is a human.
Embodiment 12. The method of embodiment 10 or 11, wherein said subject has an antibiotic resistant infection.
Embodiment 13. The method of any one of embodiments 10-12, wherein said subject has a chronic infection.
Embodiment 14. The method of any one of embodiment 10-13, wherein the said bacteria belongs to the genus Staphylococcus or Enterococcus.
Embodiment 15. The method of any one of embodiments 10-14, wherein the infection is a B. subtilis, E. coli E. faecalis, or S. aureus infection.
Embodiment 16. A method of treating Methicillin-resistant S. aureus (MRSA), low level vancomycin resistant E. faecium, or high level vancomycin resistant E. faecium said method comprising administering to said mammal a therapeutically-effective amount of a peptide of any one of embodiments 1-6.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An isolated peptide comprising an amino acid sequence comprising, consisting of, or having at least 85% identity to the amino acid sequence of a peptide of any of Tables 1-6.

2. The peptide of claim 1, wherein each amino acid in the peptide is a D-amino acid.

3. An isolated polynucleotide encoding a peptide of claim 1.

4. A vector comprising the isolated polynucleotide of claim 3.

5. A host cell comprising the vector of claim 4.

6. A method of treating a bacterial infection in a subject, said method comprising administering to said subject a therapeutically-effective amount of a peptide of claim 1.

7. The method of claim 6, wherein said subject is a human.

8. The method of claim 6, wherein said subject has an antibiotic resistant infection

9. The method of claim 6, wherein said subject has a chronic infection.

10. The method of claim 6, wherein the said bacteria belongs to the genus Staphylococcus or Enterococcus.

11. The method of claim 6, wherein the infection is a B. subtilis, E. coli E. faecalis or S. aureus infection.

12. A method of treating Methicillin-resistant S. aureus (MRSA), low level vancomycin resistant E. faecium, or high level vancomycin resistant E. faecium said method comprising administering to said mammal a therapeutically-effective amount of a peptide of claim 1.


SEQUENCE LISTING (493 sequences)

SEQ ID NO: 1 - protein, 12 aa, Artificial Sequence
  Synthetic Construct
ADWYHWRSHSSS

SEQ ID NO: 2 - protein, 12 aa, Artificial Sequence
  Synthetic Construct
AKDTHTGRMTNW

SEQ ID NO: 3 - protein, 12 aa, Artificial Sequence
  Synthetic Construct
ANVKAFFSTTQV

SEQ ID NO: 4 - protein, 12 aa, Artificial Sequence
  Synthetic Construct
APFPSVALKVPL

SEQ ID NO: 5 - protein, 12 aa, Artificial Sequence
  Synthetic Construct
ASPVNYRDMFSR

SEQ ID NO: 6 - protein, 12 aa, Artificial Sequence
  Synthetic Construct
ATNAKRTPNTRI

SEQ ID NO: 7 - protein, 12 aa, Artificial Sequence
  Synthetic Construct
ATWARVDAIARA

SEQ ID NO: 8 - protein, 12 aa, Artificial Sequence
  Synthetic Construct
CLNDSLYCQGMP

SEQ ID NO: 9 - protein, 12 aa, Artificial Sequence
  Synthetic Construct
CPLFDSVRCTSK

SEQ ID NO: 10 - protein, 12 aa, Artificial Sequence
  Synthetic Construct
DLMPHKRMIELV

SEQ ID NO: 11 - protein, 12 aa, Artificial Sequence
  Synthetic Construct
DRWVARDPASIF

SEQ ID NO: 12 - protein, 12 aa, Artificial Sequence
  Synthetic Construct
EFHPMPDGFRSR

SEQ ID NO: 13 - protein, 12 aa, Artificial Sequence
  Synthetic Construct
EGNWFLSFHAST

SEQ ID NO: 14 - protein, 12 aa, Artificial Sequence
  Synthetic Construct
FAPALSNPPLRD

SEQ ID NO: 15 - protein, 12 aa, Artificial Sequence
  Synthetic Construct
FGRMAWTPMAPM

SEQ ID NO: 16 - protein, 12 aa, Artificial Sequence
  Synthetic Construct
FHFPLGMHSRDE

SEQ ID NO: 17 - protein, 12 aa, Artificial Sequence
  Synthetic Construct
FIPFDPMSMRWE

SEQ ID NO: 18 - protein, 12 aa, Artificial Sequence
  Synthetic Construct
FYLPPNASYMSR

SEQ ID NO: 19 - protein, 12 aa, Artificial Sequence
  Synthetic Construct
GDSGLVESHRNV

SEQ ID NO: 20 - protein, 12 aa, Artificial Sequence
  Synthetic Construct
GDVSDVWTAANN

SEQ ID NO: 21 - protein, 12 aa, Artificial Sequence
  Synthetic Construct
GGGLGSLHETSM

SEQ ID NO: 22 - protein, 12 aa, Artificial Sequence
  Synthetic Construct
GGRRCRIKNCYA

SEQ ID NO: 23 - protein, 12 aa, Artificial Sequence
  Synthetic Construct
GLHTSATNLYLH

SEQ ID NO: 24 - protein, 12 aa, Artificial Sequence
  Synthetic Construct
GLPHAREDYLDL

SEQ ID NO: 25 - protein, 12 aa, Artificial Sequence
  Synthetic Construct
GLVNLMRPWHLL

SEQ ID NO: 26 - protein, 12 aa, Artificial Sequence
  Synthetic Construct
GMLQHALVPKVW

SEQ ID NO: 27 - protein, 12 aa, Artificial Sequence
  Synthetic Construct
GPFVNLRSHFES

SEQ ID NO: 28 - protein, 12 aa, Artificial Sequence
  Synthetic Construct
GQSEHHMRVASF

SEQ ID NO: 29 - protein, 12 aa, Artificial Sequence
  Synthetic Construct
GSAPLLTVDTSK

SEQ ID NO: 30 - protein, 12 aa, Artificial Sequence
  Synthetic Construct
GTGLVTLPRLTV

SEQ ID NO: 31 - protein, 12 aa, Artificial Sequence
  Synthetic Construct
GTTWVATAGKLI

SEQ ID NO: 32 - protein, 12 aa, Artificial Sequence
  Synthetic Construct
GVGLTTNVTRAG

SEQ ID NO: 33 - protein, 12 aa, Artificial Sequence
  Synthetic Construct
HHHFVTHPAWVL

SEQ ID NO: 34 - protein, 12 aa, Artificial Sequence
  Synthetic Construct
HKHWSTPEFLSS

SEQ ID NO: 35 - protein, 12 aa, Artificial Sequence
  Synthetic Construct
HKTDLWMTNTIK

SEQ ID NO: 36 - protein, 12 aa, Artificial Sequence
  Synthetic Construct
HRDPHSALTRSW

SEQ ID NO: 37 - protein, 12 aa, Artificial Sequence
  Synthetic Construct
HTSSLWHLFRST

SEQ ID NO: 38 - protein, 12 aa, Artificial Sequence
  Synthetic Construct
HVVTSSKTAGPA

SEQ ID NO: 39 - protein, 12 aa, Artificial Sequence
  Synthetic Construct
IGRTVPHQDFAR

SEQ ID NO: 40 - protein, 12 aa, Artificial Sequence
  Synthetic Construct
IPLGRDGGSYQR

SEQ ID NO: 41 - protein, 12 aa, Artificial Sequence
  Synthetic Construct
ITGLGSGSSTST

SEQ ID NO: 42 - protein, 12 aa, Artificial Sequence
  Synthetic Construct
KHFPLGMEYLVT

SEQ ID NO: 43 - protein, 12 aa, Artificial Sequence
  Synthetic Construct
KPGDTAMHYFPP

SEQ ID NO: 44 - protein, 12 aa, Artificial Sequence
  Synthetic Construct
LETRLGTSPGHT

SEQ ID NO: 45 - protein, 12 aa, Artificial Sequence
  Synthetic Construct
LPSDYRSRWADP

SEQ ID NO: 46 - protein, 12 aa, Artificial Sequence
  Synthetic Construct
MDLGYVEGSARV

SEQ ID NO: 47 - protein, 12 aa, Artificial Sequence
  Synthetic Construct
NGYHPPGFLAPE

SEQ ID NO: 48 - protein, 12 aa, Artificial Sequence
  Synthetic Construct
NHLSTPVWSITG

SEQ ID NO: 49 - protein, 12 aa, Artificial Sequence
  Synthetic Construct
QCFNSVCLHTNP

SEQ ID NO: 50 - protein, 12 aa, Artificial Sequence
  Synthetic Construct
QDIQTTPPKFSV

SEQ ID NO: 51 - protein, 12 aa, Artificial Sequence
  Synthetic Construct
QFDYMRPANDTH

SEQ ID NO: 52 - protein, 12 aa, Artificial Sequence
  Synthetic Construct
QGRIDLYGFLSH

SEQ ID NO: 53 - protein, 12 aa, Artificial Sequence
  Synthetic Construct
QITSSHVWDMGH

SEQ ID NO: 54 - protein, 12 aa, Artificial Sequence
  Synthetic Construct
QLATLHKLSGPT

SEQ ID NO: 55 - protein, 12 aa, Artificial Sequence
  Synthetic Construct
QSHYDSHLAMLV

SEQ ID NO: 56 - protein, 12 aa, Artificial Sequence
  Synthetic Construct
QVDNTSSIERLR

SEQ ID NO: 57 - protein, 12 aa, Artificial Sequence
  Synthetic Construct
RKVKRRPRVSNL

SEQ ID NO: 58 - protein, 12 aa, Artificial Sequence
  Synthetic Construct
SAGNSANWLLHW

SEQ ID NO: 59 - protein, 12 aa, Artificial Sequence
  Synthetic Construct
SFPHFTLRAYAS

SEQ ID NO: 60 - protein, 12 aa, Artificial Sequence
  Synthetic Construct
SGVYKVAYDWQH

SEQ ID NO: 61 - protein, 12 aa, Artificial Sequence
  Synthetic Construct
SINGLLSNSHGS

SEQ ID NO: 62 - protein, 12 aa, Artificial Sequence
  Synthetic Construct
SLDGAGAALRTS

SEQ ID NO: 63 - protein, 12 aa, Artificial Sequence
  Synthetic Construct
SLTVPYLTSISD

SEQ ID NO: 64 - protein, 12 aa, Artificial Sequence
  Synthetic Construct
SNSIDKVNRPIN

SEQ ID NO: 65 - protein, 12 aa, Artificial Sequence
  Synthetic Construct
SPDVTRWPYWVI

SEQ ID NO: 66 - protein, 12 aa, Artificial Sequence
  Synthetic Construct
SSSVTPVSALHG

SEQ ID NO: 67 - protein, 12 aa, Artificial Sequence
  Synthetic Construct
SVLSYSVAYSDS

SEQ ID NO: 68 - protein, 12 aa, Artificial Sequence
  Synthetic Construct
SWFSDWDLELHA

SEQ ID NO: 69 - protein, 12 aa, Artificial Sequence
  Synthetic Construct
SWHHGDGPIWYG

SEQ ID NO: 70 - protein, 12 aa, Artificial Sequence
  Synthetic Construct
TGVYWTQLNADS

SEQ ID NO: 71 - protein, 12 aa, Artificial Sequence
  Synthetic Construct
TQAIDDIIIGRI

SEQ ID NO: 72 - protein, 12 aa, Artificial Sequence
  Synthetic Construct
TTVDYFRKVWVV

SEQ ID NO: 73 - protein, 12 aa, Artificial Sequence
  Synthetic Construct
TYTANDLHLADL

SEQ ID NO: 74 - protein, 12 aa, Artificial Sequence
  Synthetic Construct
VEAKCCFSMHKT

SEQ ID NO: 75 - protein, 12 aa, Artificial Sequence
  Synthetic Construct
VFARGQYDAPHP

SEQ ID NO: 76 - protein, 12 aa, Artificial Sequence
  Synthetic Construct
VGSNLRLLHQWK

SEQ ID NO: 77 - protein, 12 aa, Artificial Sequence
  Synthetic Construct
VHWDFRQWWQPS

SEQ ID NO: 78 - protein, 12 aa, Artificial Sequence
  Synthetic Construct
VPKSVDNTFYTP

SEQ ID NO: 79 - protein, 12 aa, Artificial Sequence
  Synthetic Construct
VSHRSTANFIGS

SEQ ID NO: 80 - protein, 12 aa, Artificial Sequence
  Synthetic Construct
VSNFTKPHKPKA

SEQ ID NO: 81 - protein, 12 aa, Artificial Sequence
  Synthetic Construct
WHPRYVVSPLQY

SEQ ID NO: 82 - protein, 12 aa, Artificial Sequence
  Synthetic Construct
YAQVYSNHGSRI

SEQ ID NO: 83 - protein, 12 aa, Artificial Sequence
  Synthetic Construct
YIGEMDTLPIST

SEQ ID NO: 84 - protein, 12 aa, Artificial Sequence
  Synthetic Construct
YPDKITWQAPWL

SEQ ID NO: 85 - protein, 12 aa, Artificial Sequence
  Synthetic Construct
YSLRSDFLPFAT

SEQ ID NO: 86 - protein, 7 aa, Artificial Sequence
  Synthetic Construct
ADRFQAL

SEQ ID NO: 87 - protein, 7 aa, Artificial Sequence
  Synthetic Construct
AGKPFHF

SEQ ID NO: 88 - protein, 7 aa, Artificial Sequence
  Synthetic Construct
AHGRSRG

SEQ ID NO: 89 - protein, 7 aa, Artificial Sequence
  Synthetic Construct
AHTDWFN

SEQ ID NO: 90 - protein, 7 aa, Artificial Sequence
  Synthetic Construct
AIDFARN

SEQ ID NO: 91 - protein, 7 aa, Artificial Sequence
  Synthetic Construct
ALQPQKH

SEQ ID NO: 92 - protein, 7 aa, Artificial Sequence
  Synthetic Construct
ALSYSRG

SEQ ID NO: 93 - protein, 7 aa, Artificial Sequence
  Synthetic Construct
APKPIKL

SEQ ID NO: 94 - protein, 7 aa, Artificial Sequence
  Synthetic Construct
APTPGNV

SEQ ID NO: 95 - protein, 7 aa, Artificial Sequence
  Synthetic Construct
AQYVAVG

SEQ ID NO: 96 - protein, 7 aa, Artificial Sequence
  Synthetic Construct
ASLSKYS

SEQ ID NO: 97 - protein, 7 aa, Artificial Sequence
  Synthetic Construct
ATYGNLW

SEQ ID NO: 98 - protein, 7 aa, Artificial Sequence
  Synthetic Construct
AVRGYEW

SEQ ID NO: 99 - protein, 7 aa, Artificial Sequence
  Synthetic Construct
AYDDWFW

SEQ ID NO: 100 - protein, 7 aa, Artificial Sequence
  Synthetic Construct
DISRMAT

SEQ ID NO: 101 - protein, 7 aa, Artificial Sequence
  Synthetic Construct
DSVETKP

SEQ ID NO: 102 - protein, 7 aa, Artificial Sequence
  Synthetic Construct
DTALHSL

SEQ ID NO: 103 - protein, 7 aa, Artificial Sequence
  Synthetic Construct
DVMMPRH

SEQ ID NO: 104 - protein, 7 aa, Artificial Sequence
  Synthetic Construct
ETALIAA

SEQ ID NO: 105 - protein, 7 aa, Artificial Sequence
  Synthetic Construct
FPAWFSA

SEQ ID NO: 106 - protein, 7 aa, Artificial Sequence
  Synthetic Construct
FPITYDF

SEQ ID NO: 107 - protein, 7 aa, Artificial Sequence
  Synthetic Construct
FSTTHPD

SEQ ID NO: 108 - protein, 7 aa, Artificial Sequence
  Synthetic Construct
FSYSFQH

SEQ ID NO: 109 - protein, 7 aa, Artificial Sequence
  Synthetic Construct
FVRIHDV

SEQ ID NO: 110 - protein, 7 aa, Artificial Sequence
  Synthetic Construct
GASESYL

SEQ ID NO: 111 - protein, 7 aa, Artificial Sequence
  Synthetic Construct
GFGYNVQ

SEQ ID NO: 112 - protein, 7 aa, Artificial Sequence
  Synthetic Construct
GGGHLSR

SEQ ID NO: 113 - protein, 7 aa, Artificial Sequence
  Synthetic Construct
GHRVRFP

SEQ ID NO: 114 - protein, 7 aa, Artificial Sequence
  Synthetic Construct
GHYISAN

SEQ ID NO: 115 - protein, 7 aa, Artificial Sequence
  Synthetic Construct
GKDYMGY

SEQ ID NO: 116 - protein, 7 aa, Artificial Sequence
  Synthetic Construct
GMWHLPQ

SEQ ID NO: 117 - protein, 7 aa, Artificial Sequence
  Synthetic Construct
GNVGSVR

SEQ ID NO: 118 - protein, 7 aa, Artificial Sequence
  Synthetic Construct
GQSEKHL

SEQ ID NO: 119 - protein, 7 aa, Artificial Sequence
  Synthetic Construct
GSFWHHN

SEQ ID NO: 120 - protein, 7 aa, Artificial Sequence
  Synthetic Construct
GSPDSEF

SEQ ID NO: 121 - protein, 7 aa, Artificial Sequence
  Synthetic Construct
GVHREQI

SEQ ID NO: 122 - protein, 7 aa, Artificial Sequence
  Synthetic Construct
GVMNHTF

SEQ ID NO: 123 - protein, 7 aa, Artificial Sequence
  Synthetic Construct
HGGVRLY

SEQ ID NO: 124 - protein, 7 aa, Artificial Sequence
  Synthetic Construct
HIARLSY

SEQ ID NO: 125 - protein, 7 aa, Artificial Sequence
  Synthetic Construct
HLFTTGV

SEQ ID NO: 126 - protein, 7 aa, Artificial Sequence
  Synthetic Construct
HLKHSLL

SEQ ID NO: 127 - protein, 7 aa, Artificial Sequence
  Synthetic Construct
HLNQQNH

SEQ ID NO: 128 - protein, 7 aa, Artificial Sequence
  Synthetic Construct
HMGKLNR

SEQ ID NO: 129 - protein, 7 aa, Artificial Sequence
  Synthetic Construct
HVMTKAL

SEQ ID NO: 130 - protein, 7 aa, Artificial Sequence
  Synthetic Construct
HVRHYSD

SEQ ID NO: 131 - protein, 7 aa, Artificial Sequence
  Synthetic Construct
HYIDFRW

SEQ ID NO: 132 - protein, 7 aa, Artificial Sequence
  Synthetic Construct
IPFSFTG

SEQ ID NO: 133 - protein, 7 aa, Artificial Sequence
  Synthetic Construct
IRIAEPM

SEQ ID NO: 134 - protein, 7 aa, Artificial Sequence
  Synthetic Construct
ISTPYIG

SEQ ID NO: 135 - protein, 7 aa, Artificial Sequence
  Synthetic Construct
KCCYTLP

SEQ ID NO: 136 - protein, 7 aa, Artificial Sequence
  Synthetic Construct
KFYAHLD

SEQ ID NO: 137 - protein, 7 aa, Artificial Sequence
  Synthetic Construct
KLSMQHR

SEQ ID NO: 138 - protein, 7 aa, Artificial Sequence
  Synthetic Construct
KPPPTLD

SEQ ID NO: 139 - protein, 7 aa, Artificial Sequence
  Synthetic Construct
KTALALE

SEQ ID NO: 140 - protein, 7 aa, Artificial Sequence
  Synthetic Construct
KVKKRPD

SEQ ID NO: 141 - protein, 7 aa, Artificial Sequence
  Synthetic Construct
LAQSSIQ

SEQ ID NO: 142 - protein, 7 aa, Artificial Sequence
  Synthetic Construct
LIQGTSL

SEQ ID NO: 143 - protein, 7 aa, Artificial Sequence
  Synthetic Construct
LMPSYPR

SEQ ID NO: 144 - protein, 7 aa, Artificial Sequence
  Synthetic Construct
LPGSEQR

SEQ ID NO: 145 - protein, 7 aa, Artificial Sequence
  Synthetic Construct
LPKMYSQ

SEQ ID NO: 146 - protein, 7 aa, Artificial Sequence
  Synthetic Construct
LPNSAYV

SEQ ID NO: 147 - protein, 7 aa, Artificial Sequence
  Synthetic Construct
LPTGHFL

SEQ ID NO: 148 - protein, 7 aa, Artificial Sequence
  Synthetic Construct
LPVRLDW

SEQ ID NO: 149 - protein, 7 aa, Artificial Sequence
  Synthetic Construct
LTLGLPY

SEQ ID NO: 150 - protein, 7 aa, Artificial Sequence
  Synthetic Construct
LVMHSEN

SEQ ID NO: 151 - protein, 7 aa, Artificial Sequence
  Synthetic Construct
LVPSDKL

SEQ ID NO: 152 - protein, 7 aa, Artificial Sequence
  Synthetic Construct
MAPTHSI

SEQ ID NO: 153 - protein, 7 aa, Artificial Sequence
  Synthetic Construct
MIRGTTV

SEQ ID NO: 154 - protein, 7 aa, Artificial Sequence
  Synthetic Construct
MMVLRNQ

SEQ ID NO: 155 - protein, 7 aa, Artificial Sequence
  Synthetic Construct
MPDMTRQ

SEQ ID NO: 156 - protein, 7 aa, Artificial Sequence
  Synthetic Construct
NDLMNRA

SEQ ID NO: 157 - protein, 7 aa, Artificial Sequence
  Synthetic Construct
NDRLHTR

SEQ ID NO: 158 - protein, 7 aa, Artificial Sequence
  Synthetic Construct
NIGQDMH

SEQ ID NO: 159 - protein, 7 aa, Artificial Sequence
  Synthetic Construct
NIVSRES

SEQ ID NO: 160 - protein, 7 aa, Artificial Sequence
  Synthetic Construct
NLRLPYI

SEQ ID NO: 161 - protein, 7 aa, Artificial Sequence
  Synthetic Construct
NSIYQAW

SEQ ID NO: 162 - protein, 7 aa, Artificial Sequence
  Synthetic Construct
NSYDVQA

SEQ ID NO: 163 - protein, 7 aa, Artificial Sequence
  Synthetic Construct
NTAVPLG

SEQ ID NO: 164 - protein, 7 aa, Artificial Sequence
  Synthetic Construct
NTVANNY

SEQ ID NO: 165 - protein, 7 aa, Artificial Sequence
  Synthetic Construct
PRLPRTR

SEQ ID NO: 166 - protein, 7 aa, Artificial Sequence
  Synthetic Construct
QLAVAPS

SEQ ID NO: 167 - protein, 7 aa, Artificial Sequence
  Synthetic Construct
QLKWYHA

SEQ ID NO: 168 - protein, 7 aa, Artificial Sequence
  Synthetic Construct
QQTNWSL

SEQ ID NO: 169 - protein, 7 aa, Artificial Sequence
  Synthetic Construct
RHDIRKT

SEQ ID NO: 170 - protein, 7 aa, Artificial Sequence
  Synthetic Construct
RPTAHMA

SEQ ID NO: 171 - protein, 7 aa, Artificial Sequence
  Synthetic Construct
RTYPREK

SEQ ID NO: 172 - protein, 7 aa, Artificial Sequence
  Synthetic Construct
SAAWNKS

SEQ ID NO: 173 - protein, 7 aa, Artificial Sequence
  Synthetic Construct
SADYSAR

SEQ ID NO: 174 - protein, 7 aa, Artificial Sequence
  Synthetic Construct
SEHNGTQ

SEQ ID NO: 175 - protein, 7 aa, Artificial Sequence
  Synthetic Construct
SEVYPQK

SEQ ID NO: 176 - protein, 7 aa, Artificial Sequence
  Synthetic Construct
SFFEQVH

SEQ ID NO: 177 - protein, 7 aa, Artificial Sequence
  Synthetic Construct
SFRIGPA

SEQ ID NO: 178 - protein, 7 aa, Artificial Sequence
  Synthetic Construct
SFSQNLH

SEQ ID NO: 179 - protein, 7 aa, Artificial Sequence
  Synthetic Construct
SFVSMPE

SEQ ID NO: 180 - protein, 7 aa, Artificial Sequence
  Synthetic Construct
SHENFTS

SEQ ID NO: 181 - protein, 7 aa, Artificial Sequence
  Synthetic Construct
SHGTWTP

SEQ ID NO: 182 - protein, 7 aa, Artificial Sequence
  Synthetic Construct
SLIAHYQ

SEQ ID NO: 183 - protein, 7 aa, Artificial Sequence
  Synthetic Construct
SNMSHAT

SEQ ID NO: 184 - protein, 7 aa, Artificial Sequence
  Synthetic Construct
SPWQYTN

SEQ ID NO: 185 - protein, 7 aa, Artificial Sequence
  Synthetic Construct
SQNFVRE

SEQ ID NO: 186 - protein, 7 aa, Artificial Sequence
  Synthetic Construct
SSDVPYL

SEQ ID NO: 187 - protein, 7 aa, Artificial Sequence
  Synthetic Construct
SSLRIPV

SEQ ID NO: 188 - protein, 7 aa, Artificial Sequence
  Synthetic Construct
SSNQFHQ

SEQ ID NO: 189 - protein, 7 aa, Artificial Sequence
  Synthetic Construct
STKTLPA

SEQ ID NO: 190 - protein, 7 aa, Artificial Sequence
  Synthetic Construct
STVKYID

SEQ ID NO: 191 - protein, 7 aa, Artificial Sequence
  Synthetic Construct
SWTALGP

SEQ ID NO: 192 - protein, 7 aa, Artificial Sequence
  Synthetic Construct
TDEIKLL

SEQ ID NO: 193 - protein, 7 aa, Artificial Sequence
  Synthetic Construct
TGFLVNV

SEQ ID NO: 194 - protein, 7 aa, Artificial Sequence
  Synthetic Construct
TMQNIPN

SEQ ID NO: 195 - protein, 7 aa, Artificial Sequence
  Synthetic Construct
TQTVLGD

SEQ ID NO: 196 - protein, 7 aa, Artificial Sequence
  Synthetic Construct
TSQYLMI

SEQ ID NO: 197 - protein, 7 aa, Artificial Sequence
  Synthetic Construct
TTLLTVS

SEQ ID NO: 198 - protein, 7 aa, Artificial Sequence
  Synthetic Construct
TTQVLEA

SEQ ID NO: 199 - protein, 7 aa, Artificial Sequence
  Synthetic Construct
TVNFKLY

SEQ ID NO: 200 - protein, 7 aa, Artificial Sequence
  Synthetic Construct
TVSPRFL

SEQ ID NO: 201 - protein, 7 aa, Artificial Sequence
  Synthetic Construct
TWSLDYP

SEQ ID NO: 202 - protein, 7 aa, Artificial Sequence
  Synthetic Construct
VDSRYHP

SEQ ID NO: 203 - protein, 7 aa, Artificial Sequence
  Synthetic Construct
VHPLKLI

SEQ ID NO: 204 - protein, 7 aa, Artificial Sequence
  Synthetic Construct
VLTRCCG

SEQ ID NO: 205 - protein, 7 aa, Artificial Sequence
  Synthetic Construct
VPIYHLT

SEQ ID NO: 206 - protein, 7 aa, Artificial Sequence
  Synthetic Construct
VPVWALT

SEQ ID NO: 207 - protein, 7 aa, Artificial Sequence
  Synthetic Construct
VSGFRID

SEQ ID NO: 208 - protein, 7 aa, Artificial Sequence
  Synthetic Construct
VSQRTEP

SEQ ID NO: 209 - protein, 7 aa, Artificial Sequence
  Synthetic Construct
VSRANEG

SEQ ID NO: 210 - protein, 7 aa, Artificial Sequence
  Synthetic Construct
VSRDTPQ

SEQ ID NO: 211 - protein, 7 aa, Artificial Sequence
  Synthetic Construct
VTNTPWP

SEQ ID NO: 212 - protein, 7 aa, Artificial Sequence
  Synthetic Construct
VTSPYAF

SEQ ID NO: 213 - protein, 7 aa, Artificial Sequence
  Synthetic Construct
VYPGPSY

SEQ ID NO: 214 - protein, 7 aa, Artificial Sequence
  Synthetic Construct
WDPRVNV

SEQ ID NO: 215 - protein, 7 aa, Artificial Sequence
  Synthetic Construct
WGRISHV

SEQ ID NO: 216 - protein, 7 aa, Artificial Sequence
  Synthetic Construct
WPTHYLV

SEQ ID NO: 217 - protein, 7 aa, Artificial Sequence
  Synthetic Construct
WQEHRDQ

SEQ ID NO: 218 - protein, 7 aa, Artificial Sequence
  Synthetic Construct
WQWPARV

SEQ ID NO: 219 - protein, 7 aa, Artificial Sequence
  Synthetic Construct
WSLSELH

SEQ ID NO: 220 - protein, 7 aa, Artificial Sequence
  Synthetic Construct
WSWGEQK

SEQ ID NO: 221 - protein, 7 aa, Artificial Sequence
  Synthetic Construct
YGGAALQ

SEQ ID NO: 222 - protein, 7 aa, Artificial Sequence
  Synthetic Construct
YMLDSTM

SEQ ID NO: 223 - protein, 7 aa, Artificial Sequence
  Synthetic Construct
YNISVNK

SEQ ID NO: 224 - protein, 7 aa, Artificial Sequence
  Synthetic Construct
YPFFSSM

SEQ ID NO: 225 - protein, 7 aa, Artificial Sequence
  Synthetic Construct
YPWFIRA

SEQ ID NO: 226 - protein, 7 aa, Artificial Sequence
  Synthetic Construct
YPWWNTL

SEQ ID NO: 227 - protein, 7 aa, Artificial Sequence
  Synthetic Construct
YQWELYS

SEQ ID NO: 228 - protein, 7 aa, Artificial Sequence
  Synthetic Construct
YSEPAVT

SEQ ID NO: 229 - protein, 7 aa, Artificial Sequence
  Synthetic Construct
YSGASTL

SEQ ID NO: 230 - protein, 7 aa, Artificial Sequence
  Synthetic Construct
YYNTTPN

SEQ ID NO: 231 - protein, 9 aa, Artificial Sequence
  Synthetic Construct
CAGHNRDRC

SEQ ID NO: 232 - protein, 9 aa, Artificial Sequence
  Synthetic Construct
CAKSPMNCC

SEQ ID NO: 233 - protein, 9 aa, Artificial Sequence
  Synthetic Construct
CASKSTHDC

SEQ ID NO: 234 - protein, 9 aa, Artificial Sequence
  Synthetic Construct
CDAMIGKSC

SEQ ID NO: 235 - protein, 9 aa, Artificial Sequence
  Synthetic Construct
CDFIMGITC

SEQ ID NO: 236 - protein, 9 aa, Artificial Sequence
  Synthetic Construct
CDGHDQSLC

SEQ ID NO: 237 - protein, 9 aa, Artificial Sequence
  Synthetic Construct
CDHPHKQQC

SEQ ID NO: 238 - protein, 9 aa, Artificial Sequence
  Synthetic Construct
CDHTYTNKC

SEQ ID NO: 239 - protein, 9 aa, Artificial Sequence
  Synthetic Construct
CDKFHELQC

SEQ ID NO: 240 - protein, 9 aa, Artificial Sequence
  Synthetic Construct
CDNIMTPVC

SEQ ID NO: 241 - protein, 9 aa, Artificial Sequence
  Synthetic Construct
CDQMWHTSC

SEQ ID NO: 242 - protein, 9 aa, Artificial Sequence
  Synthetic Construct
CDRTISNKC

SEQ ID NO: 243 - protein, 9 aa, Artificial Sequence
  Synthetic Construct
CEDLTTLSC

SEQ ID NO: 244 - protein, 9 aa, Artificial Sequence
  Synthetic Construct
CEGQRWMQC

SEQ ID NO: 245 - protein, 9 aa, Artificial Sequence
  Synthetic Construct
CELGTVQSC

SEQ ID NO: 246 - protein, 9 aa, Artificial Sequence
  Synthetic Construct
CFGQGTLQC

SEQ ID NO: 247 - protein, 9 aa, Artificial Sequence
  Synthetic Construct
CFNMFSRVC

SEQ ID NO: 248 - protein, 9 aa, Artificial Sequence
  Synthetic Construct
CGDGSQRTC

SEQ ID NO: 249 - protein, 9 aa, Artificial Sequence
  Synthetic Construct
CGGGPLYMC

SEQ ID NO: 250 - protein, 9 aa, Artificial Sequence
  Synthetic Construct
CGHSNLSNC

SEQ ID NO: 251 - protein, 9 aa, Artificial Sequence
  Synthetic Construct
CGLKALKEC

SEQ ID NO: 252 - protein, 9 aa, Artificial Sequence
  Synthetic Construct
CGNSSLNRC

SEQ ID NO: 253 - protein, 9 aa, Artificial Sequence
  Synthetic Construct
CGYSSFNRC

SEQ ID NO: 254 - protein, 9 aa, Artificial Sequence
  Synthetic Construct
CHDLNGSMC

SEQ ID NO: 255 - protein, 9 aa, Artificial Sequence
  Synthetic Construct
CHNEGNRAC

SEQ ID NO: 256 - protein, 9 aa, Artificial Sequence
  Synthetic Construct
CHNRVPLMC

SEQ ID NO: 257 - protein, 9 aa, Artificial Sequence
  Synthetic Construct
CHPVSGQKC

SEQ ID NO: 258 - protein, 9 aa, Artificial Sequence
  Synthetic Construct
CHSDANSIC

SEQ ID NO: 259 - protein, 9 aa, Artificial Sequence
  Synthetic Construct
CHYNAHRTC

SEQ ID NO: 260 - protein, 9 aa, Artificial Sequence
  Synthetic Construct
CIAARHMNC

SEQ ID NO: 261 - protein, 9 aa, Artificial Sequence
  Synthetic Construct
CILLPDKCL

SEQ ID NO: 262 - protein, 9 aa, Artificial Sequence
  Synthetic Construct
CKAALTRWC

SEQ ID NO: 263 - protein, 9 aa, Artificial Sequence
  Synthetic Construct
CKDHVTRVC

SEQ ID NO: 264 - protein, 9 aa, Artificial Sequence
  Synthetic Construct
CKHLLGENC

SEQ ID NO: 265 - protein, 9 aa, Artificial Sequence
  Synthetic Construct
CKLTTQMMC

SEQ ID NO: 266 - protein, 9 aa, Artificial Sequence
  Synthetic Construct
CKMSMLHNC

SEQ ID NO: 267 - protein, 9 aa, Artificial Sequence
  Synthetic Construct
CKSMMRLNC

SEQ ID NO: 268 - protein, 9 aa, Artificial Sequence
  Synthetic Construct
CKTLQPWTC

SEQ ID NO: 269 - protein, 9 aa, Artificial Sequence
  Synthetic Construct
CLDIFSSSC

SEQ ID NO: 270 - protein, 9 aa, Artificial Sequence
  Synthetic Construct
CLHGDVAYC

SEQ ID NO: 271 - protein, 9 aa, Artificial Sequence
  Synthetic Construct
CLKLGEKWC

SEQ ID NO: 272 - protein, 9 aa, Artificial Sequence
  Synthetic Construct
CLKNQSDQC

SEQ ID NO: 273 - protein, 9 aa, Artificial Sequence
  Synthetic Construct
CLNSSQPSC

SEQ ID NO: 274 - protein, 9 aa, Artificial Sequence
  Synthetic Construct
CLRTSNPAC

SEQ ID NO: 275 - protein, 9 aa, Artificial Sequence
  Synthetic Construct
CLVSQHTDC

SEQ ID NO: 276 - protein, 9 aa, Artificial Sequence
  Synthetic Construct
CLWSTGATC

SEQ ID NO: 277 - protein, 9 aa, Artificial Sequence
  Synthetic Construct
CMAPDSRVC

SEQ ID NO: 278 - protein, 9 aa, Artificial Sequence
  Synthetic Construct
CMARYMSAC

SEQ ID NO: 279 - protein, 9 aa, Artificial Sequence
  Synthetic Construct
CMERMSLRC

SEQ ID NO: 280 - protein, 9 aa, Artificial Sequence
  Synthetic Construct
CMGFSNMSC

SEQ ID NO: 281 - protein, 9 aa, Artificial Sequence
  Synthetic Construct
CMSTGLSSC

SEQ ID NO: 282 - protein, 9 aa, Artificial Sequence
  Synthetic Construct
CMSWSLQRC

SEQ ID NO: 283 - protein, 9 aa, Artificial Sequence
  Synthetic Construct
CNAKHHPRC

SEQ ID NO: 284 - protein, 9 aa, Artificial Sequence
  Synthetic Construct
CNENIVHHC

SEQ ID NO: 285 - protein, 9 aa, Artificial Sequence
  Synthetic Construct
CNFLYSWTC

SEQ ID NO: 286 - protein, 9 aa, Artificial Sequence
  Synthetic Construct
CNHDATHTC

SEQ ID NO: 287 - protein, 9 aa, Artificial Sequence
  Synthetic Construct
CNIIHHQTC

SEQ ID NO: 288 - protein, 9 aa, Artificial Sequence
  Synthetic Construct
CYVSKNNSC

SEQ ID NO: 289 - protein, 9 aa, Artificial Sequence
  Synthetic Construct
CSEGLLNTC

SEQ ID NO: 290 - protein, 9 aa, Artificial Sequence
  Synthetic Construct
CSEHNLQTC

SEQ ID NO: 291 - protein, 9 aa, Artificial Sequence
  Synthetic Construct
CNMQITKGC

SEQ ID NO: 292 - protein, 9 aa, Artificial Sequence
  Synthetic Construct
CNPEHNNHC

SEQ ID NO: 293 - protein, 9 aa, Artificial Sequence
  Synthetic Construct
CNPTHYRSC

SEQ ID NO: 294 - protein, 9 aa, Artificial Sequence
  Synthetic Construct
CNQNASHYC

SEQ ID NO: 295 - protein, 9 aa, Artificial Sequence
  Synthetic Construct
CNQTAARVC

SEQ ID NO: 296 - protein, 9 aa, Artificial Sequence
  Synthetic Construct
CNRWHHLEC

SEQ ID NO: 297 - protein, 9 aa, Artificial Sequence
  Synthetic Construct
CNSFGVSMC

SEQ ID NO: 298 - protein, 9 aa, Artificial Sequence
  Synthetic Construct
CNTGSPYEC

SEQ ID NO: 299 - protein, 9 aa, Artificial Sequence
  Synthetic Construct
CNTTEAASC

SEQ ID NO: 300 - protein, 9 aa, Artificial Sequence
  Synthetic Construct
CPFWLSGHC

SEQ ID NO: 301 - protein, 9 aa, Artificial Sequence
  Synthetic Construct
CPRDLGTDC

SEQ ID NO: 302 - protein, 9 aa, Artificial Sequence
  Synthetic Construct
CPVALSTKC

SEQ ID NO: 303 - protein, 9 aa, Artificial Sequence
  Synthetic Construct
CPVISNGSC

SEQ ID NO: 304 - protein, 9 aa, Artificial Sequence
  Synthetic Construct
CQGNPSLRC

SEQ ID NO: 305 - protein, 9 aa, Artificial Sequence
  Synthetic Construct
CQHLRGLLC

SEQ ID NO: 306 - protein, 9 aa, Artificial Sequence
  Synthetic Construct
CQMQLRSAC

SEQ ID NO: 307 - protein, 9 aa, Artificial Sequence
  Synthetic Construct
CQNWISRFC

SEQ ID NO: 308 - protein, 9 aa, Artificial Sequence
  Synthetic Construct
CQPRNLNNC

SEQ ID NO: 309 - protein, 9 aa, Artificial Sequence
  Synthetic Construct
CQYETPRYC

SEQ ID NO: 310 - protein, 9 aa, Artificial Sequence
  Synthetic Construct
CRGATPMSC

SEQ ID NO: 311 - protein, 9 aa, Artificial Sequence
  Synthetic Construct
CRINPMSNC

SEQ ID NO: 312 - protein, 9 aa, Artificial Sequence
  Synthetic Construct
CRSANIYTC

SEQ ID NO: 313 - protein, 9 aa, Artificial Sequence
  Synthetic Construct
CRSATHSAC

SEQ ID NO: 314 - protein, 9 aa, Artificial Sequence
  Synthetic Construct
CRSQSGSNC

SEQ ID NO: 315 - protein, 9 aa, Artificial Sequence
  Synthetic Construct
CSDARSPKC

SEQ ID NO: 316 - protein, 9 aa, Artificial Sequence
  Synthetic Construct
CSGPGINLC

SEQ ID NO: 317 - protein, 9 aa, Artificial Sequence
  Synthetic Construct
CSGWQVRMC

SEQ ID NO: 318 - protein, 9 aa, Artificial Sequence
  Synthetic Construct
CSHMEYPRC

SEQ ID NO: 319 - protein, 9 aa, Artificial Sequence
  Synthetic Construct
CSISSLTHC

SEQ ID NO: 320 - protein, 9 aa, Artificial Sequence
  Synthetic Construct
CSNHRIMSC

SEQ ID NO: 321 - protein, 9 aa, Artificial Sequence
  Synthetic Construct
CSQLPWYSC

SEQ ID NO: 322 - protein, 9 aa, Artificial Sequence
  Synthetic Construct
CSSPFPEFC

SEQ ID NO: 323 - protein, 9 aa, Artificial Sequence
  Synthetic Construct
CSSVTDRWC

SEQ ID NO: 324 - protein, 9 aa, Artificial Sequence
  Synthetic Construct
CSTNSHSRC

SEQ ID NO: 325 - protein, 9 aa, Artificial Sequence
  Synthetic Construct
CSVGTNFQC

SEQ ID NO: 326 - protein, 9 aa, Artificial Sequence
  Synthetic Construct
CTERTSTEC

SEQ ID NO: 327 - protein, 9 aa, Artificial Sequence
  Synthetic Construct
CTGKNAPKC

SEQ ID NO: 328 - protein, 9 aa, Artificial Sequence
  Synthetic Construct
CTILMKILC

SEQ ID NO: 329 - protein, 9 aa, Artificial Sequence
  Synthetic Construct
CTKSLAHTC

SEQ ID NO: 330 - protein, 9 aa, Artificial Sequence
  Synthetic Construct
CTLRDSPHC

SEQ ID NO: 331 - protein, 9 aa, Artificial Sequence
  Synthetic Construct
CTNANHYFC

SEQ ID NO: 332 - protein, 9 aa, Artificial Sequence
  Synthetic Construct
CTNTNTAIC

SEQ ID NO: 333 - protein, 9 aa, Artificial Sequence
  Synthetic Construct
CTPGHTNRC

SEQ ID NO: 334 - protein, 9 aa, Artificial Sequence
  Synthetic Construct
CTPSFSKIC

SEQ ID NO: 335 - protein, 9 aa, Artificial Sequence
  Synthetic Construct
CTQMNDSFC

SEQ ID NO: 336 - protein, 9 aa, Artificial Sequence
  Synthetic Construct
CTQSSAMSC

SEQ ID NO: 337 - protein, 9 aa, Artificial Sequence
  Synthetic Construct
CTVRTSADC

SEQ ID NO: 338 - protein, 9 aa, Artificial Sequence
  Synthetic Construct
CVGMQSNTC

SEQ ID NO: 339 - protein, 9 aa, Artificial Sequence
  Synthetic Construct
CVNLQKDMC

SEQ ID NO: 340 - protein, 9 aa, Artificial Sequence
  Synthetic Construct
CVPILEGTC

SEQ ID NO: 341 - protein, 9 aa, Artificial Sequence
  Synthetic Construct
CVPMQDHTC

SEQ ID NO: 342 - protein, 9 aa, Artificial Sequence
  Synthetic Construct
CVQMPAHSC

SEQ ID NO: 343 - protein, 9 aa, Artificial Sequence
  Synthetic Construct
CYAFNYPHC

SEQ ID NO: 344 - protein, 9 aa, Artificial Sequence
  Synthetic Construct
CYGNVTNTC

SEQ ID NO: 345 - protein, 11 aa, Artificial Sequence
  Synthetic Construct
CAAHQYCWTSC

SEQ ID NO: 346 - protein, 11 aa, Artificial Sequence
  Synthetic Construct
CAEQSCIFNLC

SEQ ID NO: 347 - protein, 12 aa, Artificial Sequence
  Synthetic Construct
CALLIDCQYPLC

SEQ ID NO: 348 - protein, 11 aa, Artificial Sequence
  Synthetic Construct
CALSCHQVSLC

SEQ ID NO: 349 - protein, 12 aa, Artificial Sequence
  Synthetic Construct
CAPLCGHRVPQC

SEQ ID NO: 350 - protein, 10 aa, Artificial Sequence
  Synthetic Construct
CARACQFGAC

SEQ ID NO: 351 - protein, 10 aa, Artificial Sequence
  Synthetic Construct
CDHQCGDHLC

SEQ ID NO: 352 - protein, 12 aa, Artificial Sequence
  Synthetic Construct
CDQELCRELTSC

SEQ ID NO: 353 - protein, 12 aa, Artificial Sequence
  Synthetic Construct
CDQGDCHQKINC

SEQ ID NO: 354 - protein, 11 aa, Artificial Sequence
  Synthetic Construct
CDVPCVAQYIC

SEQ ID NO: 355 - protein, 12 aa, Artificial Sequence
  Synthetic Construct
CEKKYCTQQLPC

SEQ ID NO: 356 - protein, 12 aa, Artificial Sequence
  Synthetic Construct
CEMLQSCQQDWC

SEQ ID NO: 357 - protein, 12 aa, Artificial Sequence
  Synthetic Construct
CETRGCYQRFRC

SEQ ID NO: 358 - protein, 12 aa, Artificial Sequence
  Synthetic Construct
CFKQNCSQSRSC

SEQ ID NO: 359 - protein, 11 aa, Artificial Sequence
  Synthetic Construct
CFQLCPSVDFC

SEQ ID NO: 360 - protein, 12 aa, Artificial Sequence
  Synthetic Construct
CGAQGCFGVQSC

SEQ ID NO: 361 - protein, 11 aa, Artificial Sequence
  Synthetic Construct
CGGGICRTHNC

SEQ ID NO: 362 - protein, 10 aa, Artificial Sequence
  Synthetic Construct
CGQVCNQKVC

SEQ ID NO: 363 - protein, 11 aa, Artificial Sequence
  Synthetic Construct
CHRQLCSPSEC

SEQ ID NO: 364 - protein, 12 aa, Artificial Sequence
  Synthetic Construct
CHRTPCSLPTTC

SEQ ID NO: 365 - protein, 12 aa, Artificial Sequence
  Synthetic Construct
CIARDCWQGFSC

SEQ ID NO: 366 - protein, 12 aa, Artificial Sequence
  Synthetic Construct
CIEQPACPNIFC

SEQ ID NO: 367 - protein, 12 aa, Artificial Sequence
  Synthetic Construct
CIPIKRCNDQLC

SEQ ID NO: 368 - protein, 12 aa, Artificial Sequence
  Synthetic Construct
CISKCTSVAQSC

SEQ ID NO: 369 - protein, 12 aa, Artificial Sequence
  Synthetic Construct
CISLQQLCIRAC

SEQ ID NO: 370 - protein, 12 aa, Artificial Sequence
  Synthetic Construct
CISRVGCQNPMC

SEQ ID NO: 371 - protein, 11 aa, Artificial Sequence
  Synthetic Construct
CLPSCQHAEIC

SEQ ID NO: 372 - protein, 11 aa, Artificial Sequence
  Synthetic Construct
CLRESACSKQC

SEQ ID NO: 373 - protein, 12 aa, Artificial Sequence
  Synthetic Construct
CLRNCDYVQPPC

SEQ ID NO: 374 - protein, 10 aa, Artificial Sequence
  Synthetic Construct
CLSQFCVIDC

SEQ ID NO: 375 - protein, 12 aa, Artificial Sequence
  Synthetic Construct
CLSSGCSAQDLC

SEQ ID NO: 376 - protein, 11 aa, Artificial Sequence
  Synthetic Construct
CYQSPCPSGLC

SEQ ID NO: 377 - protein, 12 aa, Artificial Sequence
  Synthetic Construct
CLTAHCPQSISC

SEQ ID NO: 378 - protein, 12 aa, Artificial Sequence
  Synthetic Construct
CLTQPCNNPRPC

SEQ ID NO: 379 - protein, 10 aa, Artificial Sequence
  Synthetic Construct
CLVPCTQYVC

SEQ ID NO: 380 - protein, 11 aa, Artificial Sequence
  Synthetic Construct
CMRACVMQFDC

SEQ ID NO: 381 - protein, 12 aa, Artificial Sequence
  Synthetic Construct
CNDVTKLCSQFC

SEQ ID NO: 382 - protein, 12 aa, Artificial Sequence
  Synthetic Construct
CNLSCTSQTLEC

SEQ ID NO: 383 - protein, 12 aa, Artificial Sequence
  Synthetic Construct
CNLSPCLLPPQC

SEQ ID NO: 384 - protein, 12 aa, Artificial Sequence
  Synthetic Construct
CNPPHICQNPKC

SEQ ID NO: 385 - protein, 12 aa, Artificial Sequence
  Synthetic Construct
CNQRPPFCLVRC

SEQ ID NO: 386 - protein, 12 aa, Artificial Sequence
  Synthetic Construct
CPAVLSCTAEQC

SEQ ID NO: 387 - protein, 10 aa, Artificial Sequence
  Synthetic Construct
CPDQCQFSSC

SEQ ID NO: 388 - protein, 12 aa, Artificial Sequence
  Synthetic Construct
CPDTCQAAFFLC

SEQ ID NO: 389 - protein, 11 aa, Artificial Sequence
  Synthetic Construct
CPESCLDLQWC

SEQ ID NO: 390 - protein, 12 aa, Artificial Sequence
  Synthetic Construct
CPMALCSQGATC

SEQ ID NO: 391 - protein, 12 aa, Artificial Sequence
  Synthetic Construct
CPPQRRCTAFAC

SEQ ID NO: 392 - protein, 12 aa, Artificial Sequence
  Synthetic Construct
CPQLSCPSGGSC

SEQ ID NO: 393 - protein, 12 aa, Artificial Sequence
  Synthetic Construct
CPQPQPCLRTSC

SEQ ID NO: 394 - protein, 12 aa, Artificial Sequence
  Synthetic Construct
CPQSSCQGLRLC

SEQ ID NO: 395 - protein, 12 aa, Artificial Sequence
  Synthetic Construct
CPTMTVCQHPRC

SEQ ID NO: 396 - protein, 12 aa, Artificial Sequence
  Synthetic Construct
CPTSACMQQSGC

SEQ ID NO: 397 - protein, 12 aa, Artificial Sequence
  Synthetic Construct
CPVPQCDPKKLC

SEQ ID NO: 398 - protein, 12 aa, Artificial Sequence
  Synthetic Construct
CQAGVICLQQVC

SEQ ID NO: 399 - protein, 10 aa, Artificial Sequence
  Synthetic Construct
CQAVCQLGPC

SEQ ID NO: 400 - protein, 10 aa, Artificial Sequence
  Synthetic Construct
CQDGQCPRNC

SEQ ID NO: 401 - protein, 10 aa, Artificial Sequence
  Synthetic Construct
CQDLCGQMVC

SEQ ID NO: 402 - protein, 11 aa, Artificial Sequence
  Synthetic Construct
CQFHIGCYSNC

SEQ ID NO: 403 - protein, 12 aa, Artificial Sequence
  Synthetic Construct
CQHPCKSTVPNC

SEQ ID NO: 404 - protein, 12 aa, Artificial Sequence
  Synthetic Construct
CQISHCQNMIIC

SEQ ID NO: 405 - protein, 12 aa, Artificial Sequence
  Synthetic Construct
CQLINLCHDFLC

SEQ ID NO: 406 - protein, 12 aa, Artificial Sequence
  Synthetic Construct
CQLISCTGGLQC

SEQ ID NO: 407 - protein, 12 aa, Artificial Sequence
  Synthetic Construct
CQLLCVQSSSEC

SEQ ID NO: 408 - protein, 12 aa, Artificial Sequence
  Synthetic Construct
CYNQRSSCAMSC

SEQ ID NO: 409 - protein, 12 aa, Artificial Sequence
  Synthetic Construct
CQLNMCTSANNC

SEQ ID NO: 410 - protein, 12 aa, Artificial Sequence
  Synthetic Construct
CQLQLFCQTRTC

SEQ ID NO: 411 - protein, 12 aa, Artificial Sequence
  Synthetic Construct
CQPRHCIHSTVC

SEQ ID NO: 412 - protein, 12 aa, Artificial Sequence
  Synthetic Construct
CQPSTSCLIQRC

SEQ ID NO: 413 - protein, 11 aa, Artificial Sequence
  Synthetic Construct
CQPTPTCGWTC

SEQ ID NO: 414 - protein, 12 aa, Artificial Sequence
  Synthetic Construct
CQQFGQCSQFSC

SEQ ID NO: 415 - protein, 11 aa, Artificial Sequence
  Synthetic Construct
CQQRYSCFTNC

SEQ ID NO: 416 - protein, 12 aa, Artificial Sequence
  Synthetic Construct
CQQYNCVPVGRC

SEQ ID NO: 417 - protein, 12 aa, Artificial Sequence
  Synthetic Construct
CQSFSCGQRLSC

SEQ ID NO: 418 - protein, 12 aa, Artificial Sequence
  Synthetic Construct
CQSLECAMRAHC

SEQ ID NO: 419 - protein, 12 aa, Artificial Sequence
  Synthetic Construct
CQSPSLCMGLPC

SEQ ID NO: 420 - protein, 12 aa, Artificial Sequence
  Synthetic Construct
CQSPWCQRLDLC

SEQ ID NO: 421 - protein, 12 aa, Artificial Sequence
  Synthetic Construct
CQSQDHCFHKDC

SEQ ID NO: 422 - protein, 11 aa, Artificial Sequence
  Synthetic Construct
CQTDVCQRTIC

SEQ ID NO: 423 - protein, 11 aa, Artificial Sequence
  Synthetic Construct
CQYNDCDMLHC

SEQ ID NO: 424 - protein, 10 aa, Artificial Sequence
  Synthetic Construct
CRAACNPFIC

SEQ ID NO: 425 - protein, 11 aa, Artificial Sequence
  Synthetic Construct
CREVTCHHLQC

SEQ ID NO: 426 - protein, 11 aa, Artificial Sequence
  Synthetic Construct
CRPQECAQHVC

SEQ ID NO: 427 - protein, 12 aa, Artificial Sequence
  Synthetic Construct
CRQAYCSNLLLC

SEQ ID NO: 428 - protein, 11 aa, Artificial Sequence
  Synthetic Construct
CRSETCAYQDC

SEQ ID NO: 429 - protein, 12 aa, Artificial Sequence
  Synthetic Construct
CRSTPCQNQLEC

SEQ ID NO: 430 - protein, 12 aa, Artificial Sequence
  Synthetic Construct
CSHQCRSSELLC

SEQ ID NO: 431 - protein, 12 aa, Artificial Sequence
  Synthetic Construct
CSKQHTCVSPVC

SEQ ID NO: 432 - protein, 12 aa, Artificial Sequence
  Synthetic Construct
CSLITQCGGVGC

SEQ ID NO: 433 - protein, 12 aa, Artificial Sequence
  Synthetic Construct
CSMGMCALPWQC

SEQ ID NO: 434 - protein, 11 aa, Artificial Sequence
  Synthetic Construct
CSNICLAKPHC

SEQ ID NO: 435 - protein, 10 aa, Artificial Sequence
  Synthetic Construct
CSQSNCVKAC

SEQ ID NO: 436 - protein, 12 aa, Artificial Sequence
  Synthetic Construct
CSSHVICNSNSC

SEQ ID NO: 437 - protein, 12 aa, Artificial Sequence
  Synthetic Construct
CSSREQCMITVC

SEQ ID NO: 438 - protein, 11 aa, Artificial Sequence
  Synthetic Construct
CSTLDRCYQLC

SEQ ID NO: 439 - protein, 12 aa, Artificial Sequence
  Synthetic Construct
CSWYKCFNQPSC

SEQ ID NO: 440 - protein, 11 aa, Artificial Sequence
  Synthetic Construct
CYSGCGNLQGC

SEQ ID NO: 441 - protein, 12 aa, Artificial Sequence
  Synthetic Construct
CSYLCEPAQHVC

SEQ ID NO: 442 - protein, 11 aa, Artificial Sequence
  Synthetic Construct
CTAPGNCSQLC

SEQ ID NO: 443 - protein, 10 aa, Artificial Sequence
  Synthetic Construct
CTDSCPPQSC

SEQ ID NO: 444 - protein, 11 aa, Artificial Sequence
  Synthetic Construct
CTGNCVSSVGC

SEQ ID NO: 445 - protein, 12 aa, Artificial Sequence
  Synthetic Construct
CTLEICRSQLGC

SEQ ID NO: 446 - protein, 12 aa, Artificial Sequence
  Synthetic Construct
CTLNCNSGFQRC

SEQ ID NO: 447 - protein, 12 aa, Artificial Sequence
  Synthetic Construct
CTLTQCSLSKAC

SEQ ID NO: 448 - protein, 11 aa, Artificial Sequence
  Synthetic Construct
CTPLCTPQHVC

SEQ ID NO: 449 - protein, 12 aa, Artificial Sequence
  Synthetic Construct
CTQLCTASPFSC

SEQ ID NO: 450 - protein, 10 aa, Artificial Sequence
  Synthetic Construct
CTQQCPSSVC

SEQ ID NO: 451 - protein, 12 aa, Artificial Sequence
  Synthetic Construct
CTQVPCTPYQGC

SEQ ID NO: 452 - protein, 11 aa, Artificial Sequence
  Synthetic Construct
CTRDCPSQAHC

SEQ ID NO: 453 - protein, 12 aa, Artificial Sequence
  Synthetic Construct
CTRVCSSSQLYC

SEQ ID NO: 454 - protein, 12 aa, Artificial Sequence
  Synthetic Construct
CTSSLCQLSVLC

SEQ ID NO: 455 - protein, 11 aa, Artificial Sequence
  Synthetic Construct
CTTSCVKSSIC

SEQ ID NO: 456 - protein, 12 aa, Artificial Sequence
  Synthetic Construct
CTVPTCSQSLRC

SEQ ID NO: 457 - protein, 12 aa, Artificial Sequence
  Synthetic Construct
CVGQEPCLSYTC

SEQ ID NO: 458 - protein, 10 aa, Artificial Sequence
  Synthetic Construct
CVKSCGQSVC

SEQ ID NO: 459 - protein, 10 aa, Artificial Sequence
  Synthetic Construct
CVNSCSSLKC

SEQ ID NO: 460 - protein, 12 aa, Artificial Sequence
  Synthetic Construct
CVNSLCTLPSQC

SEQ ID NO: 461 - protein, 12 aa, Artificial Sequence
  Synthetic Construct
CVPSQWCYAQRC

SEQ ID NO: 462 - protein, 10 aa, Artificial Sequence
  Synthetic Construct
CVPTCSRSGC

SEQ ID NO: 463 - protein, 12 aa, Artificial Sequence
  Synthetic Construct
CVQLTCEYLYAC

SEQ ID NO: 464 - protein, 12 aa, Artificial Sequence
  Synthetic Construct
CVWQGCALNWRC

SEQ ID NO: 465 - protein, 12 aa, Artificial Sequence
  Synthetic Construct
CVYTSCVQSLTC

SEQ ID NO: 466 - protein, 10 aa, Artificial Sequence
  Synthetic Construct
CWEQACSQEC

SEQ ID NO: 467 - protein, 10 aa, Artificial Sequence
  Synthetic Construct
CWRSCPKGYC

SEQ ID NO: 468 - protein, 11 aa, Artificial Sequence
  Synthetic Construct
CYAQRCGVTGC

SEQ ID NO: 469 - protein, 12 aa, Artificial Sequence
  Synthetic Construct
CYESCRVQSALC

SEQ ID NO: 470 - protein, 12 aa, Artificial Sequence
  Synthetic Construct
CYHVRPCSSQLC

SEQ ID NO: 471 - protein, 11 aa, Artificial Sequence
  Synthetic Construct
CYMPCGQSVVC

SEQ ID NO: 472 - protein, 5 aa, Artificial Sequence
  Synthetic Construct
  MISC_FEATURE (1)..(1): Xaa is D-Ala
  MISC_FEATURE (2)..(2): Xaa is L-gamma-Glu
  MISC_FEATURE (3)..(3): Xaa is D-Lys
XXXAA

SEQ ID NO: 473 - protein, 5 aa, Artificial Sequence
  Synthetic Construct
  MISC_FEATURE (1)..(1): Xaa is UDP-MurNAc-L-Ala
  MISC_FEATURE (2)..(2): Xaa is D-gamma-Glu
  MISC_FEATURE (4)..(5): Xaa is D-Ala
XXKXX

SEQ ID NO: 474 - protein, 4 aa, Artificial Sequence
  Synthetic Construct
VSQL

SEQ ID NO: 475 - protein, 4 aa, Artificial Sequence
  Synthetic Construct
  MISC_FEATURE (1)..(1): Xaa is D-Ala
  MISC_FEATURE (2)..(2): Xaa is L-gamma-Glu
  MISC_FEATURE (3)..(3): Xaa is D-Lys
  MISC_FEATURE (4)..(4): Xaa is L-Ala-L-Lac
XXXX

SEQ ID NO: 476 - protein, 5 aa, Artificial Sequence
  Synthetic Construct
  MISC_FEATURE (1)..(1): Xaa is D-Ala
  MISC_FEATURE (3)..(3): Xaa is D-Lys
XEXAA

SEQ ID NO: 477 - protein, 14 aa, Artificial Sequence
  Synthetic Construct
  MISC_FEATURE (3)..(5): Xaa is any amino acid
  MISC_FEATURE (7)..(9): Xaa is any amino acid
ACXXXCXXXCGGSG

SEQ ID NO: 478 - protein, 15 aa, Artificial Sequence
  Synthetic Construct
  MISC_FEATURE (3)..(5): Xaa is any amino acid
  MISC_FEATURE (7)..(10): Xaa is any amino acid
ACXXXCXXXXCGGSG

SEQ ID NO: 479 - protein, 16 aa, Artificial Sequence
  Synthetic Construct
  MISC_FEATURE (3)..(5): Xaa is any amino acid
  MISC_FEATURE (7)..(11): Xaa is any amino acid
ACXXXCXXXXXCGGSG

SEQ ID NO: 480 - protein, 17 aa, Artificial Sequence
  Synthetic Construct
  MISC_FEATURE (3)..(5): Xaa is any amino acid
  MISC_FEATURE (7)..(12): Xaa is any amino acid
ACXXXCXXXXXXCGGSG

SEQ ID NO: 481 - protein, 15 aa, Artificial Sequence
  Synthetic Construct
  MISC_FEATURE (3)..(6): Xaa is any amino acid
  MISC_FEATURE (8)..(10): Xaa is any amino acid
ACXXXXCXXXCGGSG

SEQ ID NO: 482 - protein, 16 aa, Artificial Sequence
  Synthetic Construct
  MISC_FEATURE (3)..(6): Xaa is any amino acid
  MISC_FEATURE (8)..(11): Xaa is any amino acid
ACXXXXCXXXXCGGSG

SEQ ID NO: 483 - protein, 17 aa, Artificial Sequence
  Synthetic Construct
  MISC_FEATURE (3)..(6): Xaa is any amino acid
  MISC_FEATURE (8)..(12): Xaa is any amino acid
ACXXXXCXXXXXCGGSG

SEQ ID NO: 484 - protein, 18 aa, Artificial Sequence
  Synthetic Construct
  MISC_FEATURE (3)..(6): Xaa is any amino acid
  MISC_FEATURE (8)..(13): Xaa is any amino acid
ACXXXXCXXXXXXCGGSG

SEQ ID NO: 485 - protein, 16 aa, Artificial Sequence
  Synthetic Construct
  MISC_FEATURE (3)..(7): Xaa is any amino acid
  MISC_FEATURE (9)..(11): Xaa is any amino acid
ACXXXXXCXXXCGGSG

SEQ ID NO: 486 - protein, 17 aa, Artificial Sequence
  Synthetic Construct
  MISC_FEATURE (3)..(7): Xaa is any amino acid
  MISC_FEATURE (9)..(12): Xaa is any amino acid
ACXXXXXCXXXXCGGSG

SEQ ID NO: 487 - protein, 18 aa, Artificial Sequence
  Synthetic Construct
  MISC_FEATURE (3)..(7): Xaa is any amino acid
  MISC_FEATURE (9)..(13): Xaa is any amino acid
ACXXXXXCXXXXXCGGSG

SEQ ID NO: 488 - protein, 19 aa, Artificial Sequence
  Synthetic Construct
  MISC_FEATURE (3)..(7): Xaa is any amino acid
  MISC_FEATURE (9)..(14): Xaa is any amino acid
ACXXXXXCXXXXXXCGGSG

SEQ ID NO: 489 - protein, 17 aa, Artificial Sequence
  Synthetic Construct
  MISC_FEATURE (3)..(8): Xaa is any amino acid
  MISC_FEATURE (10)..(12): Xaa is any amino acid
ACXXXXXXCXXXCGGSG

SEQ ID NO: 490 - protein, 18 aa, Artificial Sequence
  Synthetic Construct
  MISC_FEATURE (3)..(8): Xaa is any amino acid
  MISC_FEATURE (10)..(13): Xaa is any amino acid
ACXXXXXXCXXXXCGGSG

SEQ ID NO: 491 - protein, 19 aa, Artificial Sequence
  Synthetic Construct
  MISC_FEATURE (3)..(8): Xaa is any amino acid
  MISC_FEATURE (10)..(14): Xaa is any amino acid
ACXXXXXXCXXXXXCGGSG

SEQ ID NO: 492 - protein, 20 aa, Artificial Sequence
  Synthetic Construct
  MISC_FEATURE (3)..(8): Xaa is any amino acid
  MISC_FEATURE (10)..(15): Xaa is any amino acid
ACXXXXXXCXXXXXXCGGSG

SEQ ID NO: 493 - protein, 9 aa, Artificial Sequence
  Synthetic Construct
CRGATMPSC